(19) United States
(12) Patent Application Publication
(10) Pub. No.: US 2008/0207645 A1
(43) Pub. Date: Aug. 28, 2008

(54) Pyridothienopyrimidine Derivatives

(76) Inventors: Luis Miguel Pages Santacana, Barcelona (ES); Joan Taltavull Moll, Barcelona (ES); Jordi Gracia Ferrer, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/791,451

(22) PCT Filed: Nov. 30, 2005
(86) PCT No.: PCT/EP05/12773

§ 371 Date: Nov. 20, 2007

(30) Foreign Application Priority Data
Nov. 30, 2004 (ES) P200402877

(51) Int. Cl. A61K 31/519; A61P 19/02

(52) U.S. Cl. 514/257

(57) ABSTRACT

A pyrido[3′,2′:4,5]thieno[3,2-d]pyrimidine derivative of formula (I)

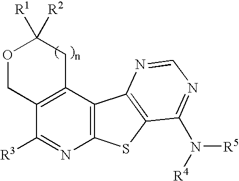

or a pharmaceutically acceptable salt or N-oxide thereof are disclosed, as well as pharmaceutical compositions comprising said compounds and methods of treatment or prevention of a pathological condition or disease susceptible to amelioration by inhibition of phosphodiesterase 4 using said compounds are disclosed.

The present invention relates to new therapeutically useful pyridothienopyrimidine derivatives, to processes for their preparation and to pharmaceutical compositions containing them. These compounds are potent and selective inhibitors of phosphodiesterase 4 (PDE4) and are thus useful in the treatment, prevention or suppression of pathological conditions, diseases and disorders known to be susceptible of being improved by inhibition of PDE4.

Phosphodiesterases (PDEs) comprise a superfamily of enzymes responsible for the hydrolysis and inactivation of the second messengers cyclic adenosine monophosphate (cAMP) and cyclic guanosine monophosphate (cGMP). Eleven different PDE families have been identified to date (PDE1 to PDE11) which differ in substrate preference, catalytic activity, sensitivity to endogenous activators and inhibitors, and encoding genes.

The PDE4 isoenzyme family exhibits a high affinity for cyclic AMP but has weak affinity for cyclic GMP. Increased cyclic AMP levels caused by PDE4 inhibition are associated with the suppression of cell activation in a wide range of inflammatory and immune cells, including lymphocytes, macrophages, basophils, neutrophils, and eosinophils. Moreover, PDE4 inhibition decreases the release of the cytokine Tumor Necrosis Factor α (TNFα). The biology of PDE4 is described in several recent reviews, for example M. D. Houslay, Prog. Nucleic Acid Res. Mol. Biol. 2001, 69, 249-315; J. E. Souness et al. Immunopharmacol. 2000 47, 127-162; or M. Conti and S. L. Jin, Prog. Nucleic Acid Res. Mol. Biol. 1999, 63, 1-38.

In view of these physiological effects, PDE4 inhibitors of varied chemical structures have been recently disclosed for the treatment or prevention of chronic and acute inflammatory diseases and of other pathological conditions, diseases and disorders known to be susceptible to amelioration by inhibition of PDE4. See, for example, U.S. Pat. No. 5,449,686, U.S. Pat. No. 5,710,170, WO 98/45268, WO 99/06404, WO 01/57025, WO 01/57036, WO 01/46184, WO 97/05105, WO 96/40636, U.S. Pat. No. 5,786,354, U.S. Pat. No. 5,773,467, U.S. Pat. No. 5,753,666, U.S. Pat. No. 5,728,712, U.S. Pat. No. 5,693,659, U.S. Pat. No. 5,679,696, U.S. Pat. No. 5,596,013, U.S. Pat. No. 5,541,219, U.S. Pat. No. 5,508,300, U.S. Pat. No. 5,502,072 or H. J. Dyke and J. G. Montana, Exp. Opin. Invest. Drugs 1999, 8, 1301-1325.

A few compounds having the capacity to selectively inhibit phosphodiesterase 4 are in active development. Examples of these compounds are cipamfylline, arofyline, cilomilast, roflumilast, mesopram and pumafentrine.

We have now found that a series of pyridothienopyrimidine derivatives are potent and selective inhibitors of PDE4 and are therefore useful in the treatment or prevention of these pathological conditions, diseases and disorders, in particular asthma, chronic obstructive pulmonary disease, rheumatoid arthritis, atopic dermatitis, psoriasis or irritable bowel disease. A number of these compounds are commercially available from libraries of compounds offered by Specs (NL), Interbioscreen Ltd. (RU) and Pharmeks (RU).

The compounds of the present invention can also be used in combination with other drugs known to be effective in the treatment of these diseases. For example, they can be used in combination with steroids or immunosuppressive agents, such as cyclosporin A, rapamycin or T-cell receptor blockers. In this case the administration of the compounds allows a reduction of the dosage of the other drugs, thus preventing the appearance of the undesired side effects associated with both steroids and immunosuppressants.

Like other PDE4 inhibitors (see references above) the compounds of the invention can also be used for blocking the ulcerogenic effects induced by a variety of etiological agents, such as antiinflammatory drugs (steroidal or non-steroidal antiinflammatory agents), stress, ammonia, ethanol and concentrated acids. They can be used alone or in combination with antacids and/or antisecretory drugs in the preventive and/or curative treatment of gastrointestinal pathologies like drug-induced ulcers, peptic ulcers, H. Pylori-related ulcers, esophagitis and gastro-esophageal reflux disease.

They can also be used in the treatment of pathological situations where damage to the cells or tissues is produced through conditions like anoxia or the production of an excess of free radicals. Examples of such beneficial effects are the protection of cardiac tissue after coronary artery occlusion or the prolongation of cell and tissue viability when the compounds of the invention are added to preserving solutions intended for storage of transplant organs or fluids such as blood or sperm. They are also of benefit on tissue repair and wound healing.

Accordingly, the present invention provides the use of the compounds of formula (I) in the manufacture of a medicament for the treatment of diseases susceptible of being improved by inhibition of PDE4; and methods of treatment of diseases susceptible to amelioration by inhibition of PDE4, which methods comprise the administration of the compounds of formula (I):

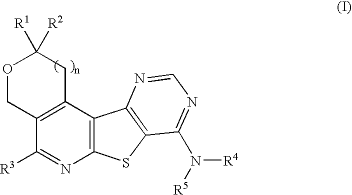

wherein
n is an integer selected from 0 or 1;
R¹ and R² are independently selected from hydrogen atoms and C₁₋₄ alkyl groups;
R³ represents a group selected from alkyl, amino, monoalkylamino, dialkylamino, aryl, heteroaryl and saturated N-containing heterocyclyl groups bound through the nitrogen atom to the piridine ring, all of them being optionally substituted by one or more substituents selected from the group consisting of halogen atoms and alkyl, alkoxyalkyl, arylalkyl, R⁶OCO—, alkoxy, R⁶R⁷N—CO—, —CN, —CF₃, —NR⁶R⁷, —SR⁶ and —SO₂NH₂ groups wherein R⁶ and R⁷ are independently selected from hydrogen atoms and C₁₋₄ alkyl groups;
R⁴ and R⁵ are independently selected from the group consisting of hydrogen atoms, alkyl groups and groups of formula (II):

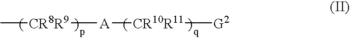

wherein p and q are integers selected from 1, 2 and 3; A is either a direct bond or a group selected from —CONR¹²—, —NR¹²CO—, —O—, —COO—, —OCO—, —NR¹²COO—, —OCONR¹²—, —NR¹²CONR¹³—, —S—, —SO—, —SO₂—, —COS— and —SCO—; and G² is a group selected from aryl heteroaryl or heterocyclyl; wherein the alkyl groups and the group G² are optionally substituted by one or more substuents selected from group consisting of halogen atoms and alkyl, alkoxyalkyl, arylalkyl, R¹⁴OCO—, hydroxy, alkoxy, oxo, R¹⁴R¹⁵N—CO—, —CN, —CF₃, —NR¹⁴R¹⁵, —SR¹⁴ and —SO₂NH₂ groups; wherein the groups R⁸ to R¹⁵ are independently selected from hydrogen atoms and C₁₋₄ alkyl groups and the pharmaceutically acceptable salts and N-oxides thereof;
to a subject in need of treatment.

Further objectives of the present invention are to provide processes for preparing said compounds and pharmaceutical compositions comprising an effective amount of said compounds.

As used herein the term alkyl embraces optionally substituted, linear or branched radicals having 1 to 20 carbon atoms or, preferably 1 to 12 carbon atoms. More preferably alkyl radicals are “lower alkyl” radicals having 1 to 8, preferably 1 to 6 and more preferably 1 to 4 carbon atoms.

Examples include methyl, ethyl, n-propyl, i-propyl, n-butyl, sec-butyl, t-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

When it is mentioned that alkyl radicals may be optionally substituted it is meant to include linear or branched alkyl, alkenyl or alkynyl radicals as defined above, which may be unsubstituted or substituted in any position by one or more substituents, for example by 1, 2 or 3 substituents. When two or more substituents are present, each substituent may be the same or different.

A said optionally substituted alkyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, substituents on an alkyl group are themselves unsubstituted. Preferred optionally substituted alkyl groups are unsubstituted or substituted with 1, 2 or 3 fluorine atoms.

As used herein, the term alkoxy (or alkyloxy) embraces optionally substituted, linear or branched oxy-containing radicals each having alkyl portions of 1 to 10 carbon atoms. More preferred alkoxy radicals are “lower alkoxy” radicals having from 1 to 8, preferably from 1 to 6 and more preferably from 1 to 4 carbon atoms.

An alkoxy group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkoxy group are themselves unsubstituted.

Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy and 2-hydroxypropoxy.

As used herein, the term monoalkylamino embraces radicals containing an optionally substituted, linear or branched alkyl radicals of 1 to 10 carbon atoms attached to a divalent —NH— radical. More preferred monoalkylamino radicals are “lower monoalkylamino” radicals having from 1 to 8, preferably from 1 to 6 and more preferably from 1 to 4 carbon atoms.

A monoalkylamino group typically contains an alkyl group which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substitutents on a monoalkylamino group are themselves unsubstituted. Preferred optionally substituted monoalkylamino radicals include methylamino, ethylamino, n-propylamino, i-propylamino, n-butylamino, sec-butylamino, t-butylamino, trifluoromethylamino, difluoromethylamino, hydroxymethylamino, 2-hydroxyethylamino and 2-hydroxypropylamino.

As used herein, the term dialkylamino embraces radicals containing a trivalent nitrogen atoms with two optionally substituted, linear or branched alkyl radicals of 1 to 10 carbon atoms attached thereto. More preferred dialkylamino radicals are “lower dialkylamino” radicals having from 1 to 8, preferably from 1 to 6 and more preferably from 1 to 4 carbon atoms in each alkyl radical.

A dialkylamino group typically contains two alkyl groups, each of which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a dialkylamino group are themselves unsubstituted.

Preferred optionally substituted dialkylamino radicals include dimethylamino, diethylamino, methyl(ethyl)amino, di(n-propyl)amino, n-propyl(methyl)amino, n-propyl(ethyl)amino, di(i-propyl)amino, i-propyl(methyl)amino, i-propyl(ethyl)amino, di(n-butyl)amino, n-butyl(methyl)amino, n-butyl(ethyl)amino, n-butyl(i-propyl)amino, di(sec-butyl)amino, sec-butyl(methyl)amino, sec-butyl(ethyl)amino, sec-butyl(n-propyl)amino, sec-butyl(i-propyl)amino, di(t-butyl)amino, t-butyl(methyl)amino, t-butyl(ethyl)amino, t-butyl(n-propyl)amino, t-butyl(i-propyl)amino, trifluoromethyl(methyl)amino, trifluoromethyl(ethyl)amino, trifluoromethyl(n-propyl)amino, trifluoromethyl(i-propyl)amino, trifluoromethyl(n-butyl)amino, trifluoromethyl(sec-butyl)amino, difluoromethyl(methyl)amino, difluoromethyl(ethyl)amino, difluoromethyl(n-propyl)amino, difluoromethyl(i-propyl)amino, difluoromethyl(n-butyl)amino, difluoromethyl(sec-butyl)amino, difluoromethyl(t-butyl)amino, difluoromethyl(trifluoromethyl)amino, hydroxymethyl(methyl)amino, ethyl(hydroxymethyl)amino, hydroxymethyl(n-propyl)amino, hydroxymethyl(i-propyl)amino, n-butyl(hydroxymethyl)amino, sec-butyl(hydroxymethyl)amino, t-butyl(hydroxymethyl)amino, difluoromethyl(hydroxymethyl)amino, hydroxymethyl(trifluoromethyl)amino, hydroxyethyl(methyl)amino, ethyl(hydroxyethyl)amino, hydroxyethyl(n-propyl)amino, hydroxyethyl(i-propyl)amino, n-butyl(hydroxyethyl)amino, sec-butyl(hydroxyethyl)amino, t-butyl(hydroxyethyl)amino, difluoromethyl(hydroxyethyl)amino, hydroxyethyl(trifluoromethyl)amino, hydroxypropyl(methyl)amino, ethyl(hydroxypropyl)amino, hydroxypropyl(n-propyl)amino, hydroxypropyl(i-propyl)amino, n-butyl(hydroxypropyl)amino, sec-butyl(hydroxypropyl)amino, t-butyl(hydroxypropyl)amino, difluoromethyl(hydroxypropyl)amino, hydroxypropyl(trifluoromethyl)amino.

As used herein, the term aryl radical embraces typically a C₅-C₁₄ monocyclic or polycyclic aryl radical such as phenyl, naphthyl, anthranyl and phenanthryl. Phenyl is preferred.

A said optionally substituted aryl radical is typically unsubstituted or substituted with 1, 2 or 3 substituents which may be the same or different. The substituents are preferably selected from halogen atoms, preferably fluorine atoms, hydroxy groups, alkoxycarbonyl groups in which the alkyl moiety has from 1 to 4 carbon atoms, hydroxycarbonyl groups, carbamoyl groups, nitro groups, cyano groups, C₁-C₄ alkyl groups, C₁-C₄ alkoxy groups and C₁-C₄ hydroxyalkyl groups. When an aryl radical carries 2 or more substituents, the substituents may be the same or different. Unless otherwise specified, the substituents on an aryl group are typically themselves unsubstituted.

As used herein, the term heteroaryl radical embraces typically a 5- to 14-membered ring system, preferably a 5- to 10-membered ring system, comprising at least one heteroaromatic ring and containing at least one heteroatom selected from O, S and N. A heteroaryl radical may be a single ring or two or more fused rings wherein at least one ring contains a heteroatom.

A said optionally substituted heteroaryl radical is typically unsubstituted or substituted with 1, 2 or 3 substituents which may be the same or different. The substituents are preferably selected from halogen atoms, preferably fluorine, chlorine or bromine atoms, alkoxycarbonyl groups in which the alkyl moiety has from 1 to 4 carbon atoms, nitro groups, hydroxy groups, C₁-C₄ alkyl groups and C₁-C₄ alkoxy groups. When an heteroaryl radical carries 2 or more substituents, the substituents may be the same or different. Unless otherwise specified, the substituents on a heteroaryl radical are typically themselves unsubstituted.

Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidnyl and the various pyrrolopyridyl radicals.

Oxadiazolyl, oxazolyl, pyridyl, pyrrolyl, imidazolyl, thiazolyl, thiadiazolyl, thienyl, furanyl, quinolinyl, isoquinolinyl, indolyl, benzoxazolyl, naphthyridinyl, benzofuranyl, pyrazinyl, pyrimidinyl and the various pyrrolopyridyl radicals are preferred.

As used herein, the term heterocyclyl radical embraces typically a non-aromatic, saturated or unsaturated C₃-C₁₀ carbocyclic ring, such as a 5, 6 or 7 membered radical, in which one or more, for example 1, 2, 3 or 4 of the carbon atoms preferably 1 or 2 of the carbon atoms are replaced by a heteroatom selected from N, O and S. Saturated heterocyclyl radicals are preferred. A heterocyclic radical may be a single ring or two or more fused rings wherein at least one ring contains a heteroatom. When a heterocyclyl radical carries 2 or more substituents, the substituents may be the same or different. A N-containing heterocyclyl radical is an heterocyclyl radical in which at least one carbon atom of the carbocyclyl ring is replaced by a nitrogen atom.

A said optionally substituted heterocyclyl radical is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a heterocyclyl radical are themselves unsubstituted.

Examples of heterocyclic radicals include piperidyl, pyrrolidyl, pyrrolinyl, piperazinyl, morpholinyl, thiomorpholinyl, pyrrolyl, pyrazolinyl, pirazolidinyl, quinuclidinyl, triazolyl, pyrazolyl, tetrazolyl, cromanyl, isocromanyl, imidazolidinyl, imidazolyl, oxiranyl, azaridinyl, 4,5-dihydro-oxazolyl and 3-aza-tetrahydrofuranyl. Preferred heterocyclyl radicals are selected from piperidyl, pyrrolidyl, piperazinyl, morpholinyl and thiomorpholinyl.

Where a heterocyclyl radical carries 2 or more substituents, the substituents may be the same or different.

As used herein, some of the atoms, radicals, moieties, chains and cycles present in the general structures of the invention are “optionally substituted”. This means that these atoms, radicals, moieties, chains and cycles can be either unsubstituted or substituted in any position by one or more, for example 1, 2, 3 or 4, substituents, whereby the hydrogen atoms bound to the unsubstituted atoms, radicals, moieties, chains and cycles are replaced by chemically acceptable atoms, radicals, moieties, chains and cycles. When two or more substituents are present, each substituent may be the same or different. The substituents are typically themselves unsubstituted.

As used herein, the term halogen atom embraces chlorine, fluorine, bromine and iodine atoms. A halogen atom is typically a fluorine, chlorine or bromine atom, most preferably chlorine or fluorine. The term halo when used as a prefix has the same meaning. Compounds containing one or more chiral centre may be used in enantiomerically or diastereoisomerically pure form, or in the form of a mixture of isomers.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

As used herein, an N-oxide is formed from the tertiary basic amines or imines present in the molecule, using a convenient oxidising agent.

In one embodiment the present invention provides the use of the compounds of formula (I) in the manufacture of a medicament for the treatment of diseases susceptible of being improved by inhibition of PDE4; and methods of treatment of diseases susceptible to amelioration by inhibition of PDE4, which methods comprise the administration of the compounds of formula (I):

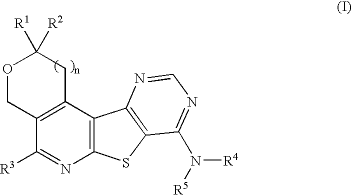

wherein
n is an integer selected from 0 or 1;
R¹ and R² are independently selected from hydrogen atoms and C₁₋₄ alkyl groups;
R³ represents a group selected from alkyl, amino, monoalkylamino, dialkylamino, aryl, heteroaryl and saturated N-containing heterocyclyl groups bound through the nitrogen atom to the piridine ring, all of them being optionally substituted by one or more substituents selected from the group consisting of halogen atoms and alkyl, alkoxyalkyl, arylalkyl, R⁶OCO—, alkoxy, R⁶R⁷N—CO—, —CN, —CF₃, —NR⁶R⁷, —SR⁶ and —SO₂NH₂ groups wherein R⁶ and R⁷ are independently selected from hydrogen atoms and C₁₋₄ alkyl groups;
R⁴ and R⁵ are independently selected from the group consisting of hydrogen atoms, alkyl groups and groups of formula (II):

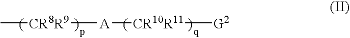

wherein p and q are integers selected from 1, 2 and 3; A is either a direct bond or a group selected from —CONR¹²—, —NR¹²CO—, —O—, —COO—, —OCO—, —NR¹²COO—, —OCONR¹²—, —NR¹²CONR¹³—, —S—, —SO—, —SO₂—, —COS— and —SCO—; and G² is a group selected from aryl heteroaryl or heterocyclyl; wherein the alkyl groups and the group G² are optionally substituted by one or more substuents selected from group consisting of halogen atoms and alkyl, alkoxyalkyl, arylalkyl, R¹⁴OCO—, alkoxy, R¹⁴R¹⁵N—CO—, —CN, —CF₃, —NR¹⁴R¹⁵, —SR¹⁴ and —SO₂NH₂ groups; wherein the groups R⁸ to R¹⁵ are independently selected from hydrogen atoms and C₁₋₄ alkyl groups
and the pharmaceutically acceptable salts and N-oxides thereof;
to a subject in need of treatment.

It is one embodiment of the present invention the use of the compounds of formula (I) wherein R¹ and R² are both methyl groups in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

It is still another embodiment of the present invention the use of the compounds of formula (I) wherein n has the value of 1; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

It is also another embodiment of the present invention the use of the compounds of formula (I) wherein R³ is selected from monoalkylamino, dialkylamino and saturated N-containing heterocyclyl groups bound through the nitrogen atom to the piridine ring, all of them being optionally substituted by one or more substituents selected from the group consisting of halogen atoms and alkyl, alkoxyalkyl, arylalkyl, R⁶OCO—, alkoxy, R⁶R⁷N—CO—, —CN, —CF₃, —NR⁶R⁷, —SR⁶ and —SO₂NH₂ groups wherein R⁶ and R⁷ are independently selected from hydrogen atoms and C₁₋₄ alkyl groups; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

It is still another embodiment of the present invention the use of the compounds of formula (I) wherein R³ is selected from monoalkylamino, dialkylamino and saturated N-containing heterocyclyl groups bound through the nitrogen atom to the piridine ring, all of them being non substituted; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease. It is still another embodiment of the present invention the use of the compounds of formula (I) wherein R⁴ is a hydrogen atom; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

It is another embodiment of the present invention the use of a compound of formula (I) wherein R⁵ is a group of formula (III)

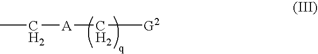

wherein q is an integer selected from 1 or 2, A represents a direct bond or a group —CONH— and G² is a group selected from aryl, heteroaryl or heterocyclyl; wherein the group G² is optionally substituted by one or more substuents selected from group consisting of halogen atoms and alkyl, alkoxyalkyl, arylalkyl, R¹⁴OCO—, alkoxy, R¹⁴R¹⁵N—CO—, —CN, —CF₃, —NR¹⁴R¹⁵, —SR¹⁴ and —SO₂NH₂ groups; wherein R¹⁴ and R¹⁵ are as hereinabove defined; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

It is yet another embodiment of the present invention to use the compounds of formula (I) wherein R⁵ is a group of formula (III)

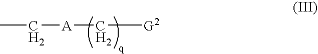

wherein q is an integer selected from 1 or 2, A represents a direct bond or a group —CONH— and G² is optionally substituted by one or more substuents selected from group consisting of halogen atoms and alkoxy and R¹⁴OCO— groups; wherein R¹⁴ is as hereinabove defined; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

It is a particularly preferred embodiment of the present invention to use the compounds of formula (I) wherein R¹ and R² are both hydrogen atoms, n has the value of 1, R³ is selected from monoalkylamino, dialkylamino and saturated N-containing heterocyclyl groups bound through the nitrogen atom to the piridine ring, all of them being non substituted, R⁴ is a hydrogen atom and R⁵ is a group of formula (III)

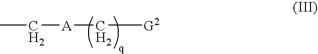

wherein q is an integer selected from 1 or 2, A represents a direct bond or a group —CONH— and G² is a group selected from aryl, heteroaryl or heterocyclyl; wherein the group G² is optionally substituted by one or more substuents selected from group consisting of halogen atoms and alkoxy and R¹⁴OCO— groups; wherein R¹⁴ is as hereinabove defined; in the manufacture of a medicament for the treatment of diseases susceptible of being improved by inhibition of PDE4, in particular for the treatment or prevention of a disorder which is selected from asthma, chronic obstructive pulmonary disease, rheumatoid arthritis, atopic dermatitis, psoriasis or irritable bowel disease.

Particular individual compounds of the invention for use as inhibitors of phosphodiesterase 4 include:
2,2-Dimethyl-5-morpholin-4-yl-N-(2-phenetylethyl)-1,4-dihydro-2H-pyrano[4″,3″:4′, 5′]pyrido[3′,2′:4,5]thieno[3,2-d]pyrimidin-8-amine
2,2-Dimethyl-5-morpholin-4-yl-N-(4-methylpiperidin-1-yl)-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(2-diethylaminoethyl)-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-butyl-N-methyl-1,4-dihydro-2H-pyrano[4″,3″:4′, 5′]pyrido[3′,2′:4,5]thieno[3,2-d]pyrimidin-8-amine
2,2-Dimethyl-5-morpholin-4-yl-N-(2-tetrahydrofuryllmethyl)-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(2-tetrahydrofurylmethyl)-1,4-dihydro-2H-pyrano[4″,3″:4′, 5′]pyrido[3′,2′:4,5]thieno[3,2-d]pyrimidin-8-amine
2,2-Dimethyl-5-morpholin-4-yl-N-butyl-1,4-dihydro-2H-pyrano[4″, 3″:4′, 5′]pyrido[3′,2′:4,5]thieno[3,2-d]pyrimidin-8-amine
2,2-Dimethyl-5-morpholin-4-yl-N-(3-diethylaminopropyl)-1,4-dihydro-2H-pyrano[4″,3″:4′,5′]pyrido[3′,2′:4,5]thieno[3,2-d]pyrimidin-8-amine
2,2-Dimethyl-5,8-dimorpholin-4-yl-1,4-dihydro-2H-pyrano[4″,3″:4′, 5′]pyrido[3′,2′:4,5]thieno[3,2-d]pyrimidine
2,2-Dimethyl-5-morpholin-4-yl-N-cyclohexyl-1,4-dihydro-2H-pyrano[4″, 3″:4′, 5′]pyrido[3′,2′:4,5]thieno[3,2-d]pyrimidin-8-amine
2,2-Dimethyl-5-morpholin-4-yl-N,N-diethyl-1,4-dihydro-2H-pyrano[4″, 3″:4′,5′]pyrido[3′,2′:4,5]thieno[3,2-d]pyrimidin-8-amine
2,2-Dimethyl-5-morpholin-4-yl-8-(2-phenylhydrazino)-1,4-dihydro-2H-pyrano[4″,3″:4′,5′]pyrido[3′,2′:4,5]thieno[3,2-d]pyrimidine
2,2-Dimethyl-5-morpholin-4-yl-N-pentyl-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-1,4-dihydro-2H-pyrano[4″,3″:4′, 5′]pyrido[3′, 2′:4,5]thieno[3,2-d]pyrimidin-8-amine
2,2-Dimethyl-5-morpholin-4-yl-N-allyl-1,4-dihydro-2H-pyrano[4″, 3″:4′, 5′]pyrido[3′,2′:4,5]thieno[3,2-d]pyrimidin-8-amine
2,2-Dimethyl-5-propyl-N-(3-hydroxypropyl)-1,4-dihydro-2H-pyrano[4″,3″:4′, 5′]pyrido[3′,2′:4,5]thieno[3,2-d]pyrimidin-8-amine
2,2-Dimethyl-5-morpholin-4-yl-N-(3-hydroxypropyl)-1,4-dihydro-2H-pyrano[4″,3″:4′, 5′]pyrido[3′,2′:4,5]thieno[3,2-d]pyrimidin-8-amine
2,2-Dimethyl-5-butyl-4-yl-N-(2-morpholin-4-ylethyl)-1,4-dihydro-2H-pyrano[4″,3″:4′, 5′]pyrido[3′,2′:4,5]thieno[3,2-d]pyrimidin-8-amine
2,2-Dimethyl-5-phenyl-4-yl-N-(2-dimethylaminoethyl)-1,4-dihydro-2H-pyrano[4″,3″:4′, 5′]pyrido[3′,2′:4,5]thieno[3,2-d]pyrimidin-8-amine
2,2-Dimethyl-5-morpholin-4-yl-N-(pyridin-2-yl)-1,4-dihydro-2H-pyrano[4″, 3″:4′, 5′]pyrido[3′ 2′:4,5]thieno[3,2-d]pyrimidin-8-amine
2,2-Dimethyl-5-morpholin-4-yl-N-(2-morpholin-4-ylethyl)-1,4-dihydro-2H-pyrano[4″,3″:4′, 5′]pyrido[3′,2′:4,5]thieno[3,2-d]pyrimidin-8-amine
2,2-Dimethyl-N-(1-methyl-3-phenylpropyl)-5-morpholin-4-yl-1,4-dihydro-2H-pyrano[4″,3″:4′,5′]pyrido[3′,2′:4,5]thieno[3,2-d]pyrimidin-8-amine
2,2-Dimethyl-5-isobutyl-4-yl-N-(2-morpholin-4-ylethyl)-1,4-dihydro-2H-pyrano[4″,3″:4′,5′]pyrido[3′,2′:4,5]thieno[3,2-d]pyrimidin-8-amine
2,2-Dimethyl-5-furan-2-yl-N-(2-morpholin-4-ylethyl)-1,4-dihydro-2H-pyrano[4″, 3″:4′,5′]pyrido[3′,2′:4,5]thieno[3,2-d]pyrimidin-8-amine
2,2-Dimethyl-5-pyrrolidin-1-yl-N-benzyl-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-benzyl-N-methyl-1,4-dihydro-2H-pyrano[4″, 3″:4′,5′]pyrido[3′,2′:4,5]thieno[3,2-d]pyrimidin-8-amine
2,2-Dimethyl-5-pyrrolidin-1-yl-8-morpholin-4-yl-1,4-dihydro-2H-pyrano[4″, 3″:4′,5′]pyrido[3′,2′:4,5]thieno[3,2-d]pyrimidine
2,2-Dimethyl-5-pyrrolidin-1-yl-N-(2-morpholin-4-ylethyl)-1,4-dihydro-2H-pyrano[4″,3″:4′, 5′]pyrido[3′,2′:4,5]thieno[3,2-d]pyrimidin-8-amine
2,2-Dimethyl-5-morpholin-4-yl-N-furan-2-ylmethyl-1,4-dihydro-2H-pyrano[4″, 3″:4′,5′]pyrido[3′,2′:4,5]thieno[3,2-d]pyrimidin-8-amine
2,2-Dimethyl-5-pyrrolidin-1-yl-N-phenetyl-1,4-dihydro-2H-pyrano[4″, 3″:4′, 5′]pyrido[3′,2′:4,5]thieno[3,2-d]pyrimidin-8-amine
2,2-Dimethyl-5-pyrrolidin-1-yl-N-(3-dimethylaminopropyl)-1,4-dihydro-2H-pyrano[4″,3″:4′,5′]pyrido[3′,2′:4,5]thieno[3,2-d]pyrimidin-8-amine
2,2-Dimethyl-5-pyrrolidin-1-yl-N-isopentyl-1,4-dihydro-2H-pyrano[4″, 3″:4′,5′]pyrido[3′,2′:4,5]thieno[3,2-d]pyrimidin-8-amine
2,2-Dimethyl-N-(1-methyl-3-phenylpropyl)-5-pyrrolidin-1-yl-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(2-hydroxyethyl)-N-benzyl-1,4-dihydro-2H-pyrano[4″, 3″:4′,5′]pyrido[3′,2′:4,5]thieno[3,2-d]pyrimidin-8-amine
2,2-Dimethyl-5-pyrrolidin-1-yl-N-tetrahydrofuran-2-yl-1,4-dihydro-2H-pyrano[4″,3″:4′,5′]pyrido[3′,2′:4,5]thieno[3,2-d]pyrimidin-8-amine
2,2-Dimethyl-5-pyrrolidin-1-yl-N-pentyl-1,4-dihydro-2H-pyrano[4″,3″:4′,5′]pyrido[3′,2′:4,5]thieno[3,2-d]pyrimidin-8-amine
2,2-dimethyl-5-morpholin-4-yl-N-(pyridin-3-ylmethyl)-1,4-dihydro-2H-pyrano[4″, 3″:4′, 5′]pyrido[3′,2′:4,5]thieno[3,2-d]pyrimidin-8-amine
2,2-dimethyl-5-morpholin-4-yl-N-(pyridin-2-ylmethyl)-1,4-dihydro-2H-pyrano[4″, 3″:4′, 5′]pyrido[3′,2′:4,5]thieno[3,2-d]pyrimidin-8-amine
2,2-dimethyl-N-(2-morpholin-4-ylethyl)-5-propyl-1,4-dihydro-2H-pyrano[4″,3″:4′,5′]pyrido[3′,2′:4,5]thieno[3,2-d]pyrimidin-8-amine
2-ethyl-2-methyl-5-morpholin-4-yl-N-(2-morpholin-4-ylethyl)-1,4-dihydro-2H-pyrano[4″,3″:4′, 5′]pyrido[3′,2′:4,5]thieno[3,2-d]pyrimidin-8-amine
2,2-dimethyl-N-(pyridin-3-ylmethyl)-5-pyrrolidin-1-yl-1,4-dihydro-2H-pyrano[4″,3″:4′, 5′]pyrido[3′,2′:4,5]thieno[3,2-d]pyrimidin-8-amine
2,2-dimethyl-N-(pyridin-2-ylmethyl)-5-pyrrolidin-1-yl-1,4-dihydro-2H-pyrano[4″,3″:4′, 5′]pyrido′[3′,2′:4,5]thieno[3,2-d]pyrimidin-8-amine
5-(2-Furyl)-2,2-dimethyl-N-(pyridin-3-ylmethyl)-1,4-dihydro-2H-pyrano[4″, 3″:4′, 5′]pyrido[3′,2′:4,5]thieno[3,2-d]pyrimidin-8-amine
5-(2-Furyl)-N-(2-furylmethyl)-2,2-dimethyl-1,4-dihydro-2H-pyrano[4″, 3″:4′, 5′]pyrido[3′,2′:4,5]thieno[3,2-d]pyrimidin-8-amine
2,2-Dimethyl-5-methyl-N-(pyridin-3-ylmethyl)-1,4-dihydro-2H-pyrano[4″, 3″:4′, 5′]pyrido[3′,2′:4,5]thieno[3,2-d]pyrimidin-8-amine
2,2-Dimethyl-5-isobutyl-N-(2-furylmethyl)-1,4-dihydro-2H-pyrano[4″, 3″:4′,5′]pyrido[3′,2′:4,5]thieno[3,2-d]pyrimidin-8-amine
2,2-Dimethyl-5-isopropyl-N-(pyridin-2-ylmethyl)-1,4-dihydro-2H-pyrano[4″, 3″:4′,5′]pyrido[3′,2′:4,5]thieno[3,2-d]pyrimidin-8-amine
2,2-Dimethyl-5-isopropyl-N-(2-furylmethyl)-1,4-dihydro-2H-pyrano[4″,3″:4′,5′]pyrido[3′,2′:4,5]thieno[3,2-d]pyrimidin-8-amine
2,2-Dimethyl-5-isopropyl-N-(pyridin-3-ylmethyl)-1,4-dihydro-2H-pyrano[4″,3″:4′,5′]pyrido[3′,2′:4,5]thieno[3,2-d]pyrimidin-8-amine
2,2-Dimethyl-5-methyl-N-(pyridin-2-ylmethyl)-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(3-morpholin-4-ylpropyl)-1,4-dihydro-2H-pyrano[4″, 3″:4′,5′]pyrido[3′,2′:4,5]thieno[3,2-d]pyrimidin-8-amine
N-[2-(3,4-Dimethoxyphenyl)ethyl]-2,2-dimethyl-5-morpholin-4-yl-1,4-dihydro-2H-pyrano[4″,3″:4′,5′]pyrido[3′,2′:4,5]thieno[3,2-d]pyrimidin-8-amine
5-(2-Furyl)-2,2-dimethyl-N-(pyridin-2-ylmethyl)-1,4-dihydro-2H-pyrano[4″,3″:4′, 5′]pyrido[3′,2′:4,5]thieno[3,2-d]pyrimidin-8-amine
N-[2-(3,4-Dimethoxyphenyl)ethyl]-2,2-dimethyl-5-isopropyl-1,4-dihydro-2H-pyrano[4″,3″:4′,5′]pyrido[3′,2′:4,5]thieno[3,2-d]pyrimidin-8-amine
1-[(5-Isopropyl-2,2-dimethyl-1,4-dihydro-2H-pyrano[4″,3″:4′,5′]pyrido[3′,2′:4,5]thieno[3,2-d]pyrimidin-8-yl)amino]propan-2-ol
2,2-Dimethyl-5-morpholin-4-yl-N-(pyridin-4-ylmethyl)-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(2-piperidin-1-ylethyl)-1,4-dihydro-2H-pyrano[4″, 3″:4′,5′]pyrido[3′,2′:4,5]thieno[3,2-d]pyrimidin-8-amine
N-(3-Methoxypropyl)-2,2-dimethyl-5-morpholin-4-yl-1,4-dihydro-2H-pyrano[4″,3″:4′,5′]pyrido[3′,2′:4,5]thieno[3,2-d]pyrimidin-8-amine
N-(2-Methoxyethyl)-N,2,2-trimethyl-N-(2-morpholin-4-ylethyl)-1,4-dihydro-2H-pyrano[4″, 3″:4′,5′]pyrido[3′,2′:4,5]thieno[3,2-d]pyrimidine-5,8-diamine.
N-(2-Methoxyethyl)-N,2,2-trimethyl-N-(pyridin-3-ylmethyl)-1,4-dihydro-2H-pyrano[4″,3″:4′, 5′]pyrido[3′,2′:4,5]thieno[3,2-d]pyrimidine-5,8-diamine.
2,2-Dimethyl-5-(4-methylpiperazin-1-yl)-N-(2-morpholin-4-ylethyl)-1,4-dihydro-2H-pyrano[4″,3″:4′,5′]pyrido[3′,2′:4,5]thieno[3,2-d]pyrimidin-8-amine.
2,2-Dimethyl-5-(4-methylpiperazin-1-yl)-N-(3-morpholin-4-ylpropyl)-1,4-dihydro-2H-pyrano[4″,3″:4′, 5′]pyrido[3′,2′:4,5]thieno[3,2-d]pyrimidin-8-amine
N-(2-Furylmethyl)-2,2-dimethyl-5-(4-methylpiperazin-1-yl)-1,4-dihydro-2H-pyrano[4″,3″:4′,5′]pyrido[3′,2′:4,5]thieno[3,2-d]pyrimidin-8-amine
2,2-Dimethyl-5-(4-methylpiperazin-1-yl)-N-(pyridin-4-ylmethyl)-1,4-dihydro-2H-pyrano[4″, 3″:4′,5′]pyrido[3′,2′:4,5]thieno[3,2-d]pyrimidin-8-amine
2,2-Dimethyl-5-(4-methylpiperazin-1-yl)-N-(pyridin-3-ylmethyl)-1,4-dihydro-2H-pyrano[4″, 3″:4′,5′]pyrido[3′,2′:4,5]thieno[3,2-d]pyrimidin-8-amine
2,2-Dimethyl-5-(4-methylpiperazin-1-yl)-N-(pyridin-2-ylmethyl)-1,4-dihydro-2H-pyrano[4″, 3″:4′, 5′]pyrido[3′,2′:4,5]thieno[3,2-d]pyrimidin-8-amine
2,2-Dimethyl-5-(4-methylpiperazin-1-yl)-N-(2-pyridin-2-ylethyl)-1,4-dihydro-2H-pyrano[4″,3″:4′,5′]pyrido[3′,2′:4,5]thieno[3,2-d]pyrimidin-8-amine
N-[3-(1H-Imidazol-1-yl)propyl]-2,2-dimethyl-5-(4-methylpiperazin-1-yl)-1,4-dihydro-2H-pyrano[4″,3″:4′,5′]pyrido[3′,2′:4,5]thieno[3,2-d]pyrimidin-8-amine
2,2-Dimethyl-5-(4-methylpiperazin-1-yl)-N-[1-(tetrahydrofuran-3-ylmethyl)piperidin-4-yl]-1,4-dihydro-2H-pyrano[4″,3″:4′,5′]pyrido[3′,2′:4,5]thieno[3,2-d]pyrimidin-8-amine
2,2-Dimethyl-N-(2-morpholin-4-ylethyl)-5-piperidin-1-yl-1,4-dihydro-2H-pyrano[4″,3″:4′,5′]pyrido[3′,2′:4,5]thieno[3,2-d]pyrimidin-8-amine
2,2-Dimethyl-5-piperidin-1-yl-N-(pyridin-3-ylmethyl)-1,4-dihydro-2H-pyrano[4″,3″:4′,5′]pyrido[3′,2′:4,5]thieno[3,2-d]pyrimidin-8-amine
2,2-Dimethyl-N-(pyridin-4-ylmethyl)-5-pyrrolidin-1-yl-1,4-dihydro-2H-pyrano[4″,3″:4′,5′]pyrido[3′,2′:4,5]thieno[3,2-d]pyrimidin-8-amine
2,2-Dimethyl-5-propyl-N-(pyridin-3-ylmethyl)-1,4-dihydro-2H-pyrano[4″,3″:4′,5′]pyrido[3′,2′:4,5]thieno[3,2-d]pyrimidin-8-amine
5-Butyl-N-(2-furylmethyl)-2,2-dimethyl-1,4-dihydro-2H-pyrano[4″,3″:4′, 5′]pyrido[3′,2′:4,5]thieno[3,2-d]pyrimidin-8-amine
5-Isobutyl-2,2-dimethyl-N-(pyridin-3-ylmethyl)-1,4-dihydro-2H-pyrano[4″,3″:4′,5′]pyrido[3′,2′:4,5]thieno[3,2-d]pyrimidin-8-amine
5-Morpholin-4-yl-N-(2-morpholin-4-ylethyl)-1,4-dihydro-2H-pyrano[4″,3″:4′,5′]pyrido[3′,2′:4,5]thieno[3,2-d]pyrimidin-8-amine
5-Morpholin-4-yl-N-pentyl-1,4-dihydro-2H-pyrano[4″, 3″:4′,5′]pyrido[3′,2′:4,5]thieno[3,2-d]pyrimidin-8-amine
N-(2-Morpholin-4-ylethyl)-5-pyrrolidin-1-yl-1,4-dihydro-2H-pyrano[4″, 3″:4′,5′]pyrido[3′,2′:4,5]thieno[3,2-d]pyrimidin-8-amine
N-Pentyl-5-pyrrolidin-1-yl-1,4-dihydro-2H-pyrano[4″, 3″:4′,5′]pyrido[3′,2′:4,5]thieno[3,2-d]pyrimidin-8-amine
N-Benzyl-5-pyrrolidin-1-yl-1,4-dihydro-2H-pyrano[4″,3″:4′,5′]pyrido[3′,2′:4,5]thieno[3,2-d]pyrimidin-8-amine
2-Ethyl-2-methyl-5-morpholin-4-yl-N-(pyridin-3-ylmethyl)-1,4-dihydro-2H-pyrano[4″,3″:4′,5′]pyrido[3′,2′:4,5]thieno[3,2-d]pyrimidin-8-amine
N⁵, N⁵,2,2-tetramethyl-N⁸-(2-morpholin-4-ylethyl)-1,4-dihydro-2H-pyrano[4″, 3″:4′, 5′]pyrido[3′,2′:4,5]thieno[3,2-d]pyrimidine-5,8-diamine
2,2-Dimethyl-5-dimethylamino-N-(3-morpholin-4-ylpropyl)-1,4-dihydro-2H-pyrano[4″,5″:4′,5′]pyrido[3′,2′:4,5]thieno[3,2-d]pyrimidin-8-amine
N⁸-(2,3-Dimethoxybenzyl)-N⁵, N⁵,2,2-tetramethyl-1,4-dihydro-2H-pyrano[4″,3″:4′, 5′]pyrido[3′,2′:4,5]thieno[3,2-d]pyrimidine-5,8-diamine
N⁵, N⁵,2,2-Tetramethyl-N⁸-(pyridin-4-ylmethyl)-1,4-dihydro-2H-pyrano[4″,3″:4′, 5′]pyrido[3′,2′:4,5]thieno[3,2-d]pyrimidine-5,8-diamine
N⁵, N⁵,2,2-Tetramethyl-N⁸-(pyridin-3-ylmethyl)-1,4-dihydro-2H-pyrano[4″,3″:4′, 5′]pyrido[3′,2′:4,5]thieno[3,2-d]pyrimidine-5,8-diamine
N⁵, N⁵,2,2-Tetramethyl-N⁸-(pyridin-2-ylmethyl)-1,4-dihydro-2H-pyrano[4″, 3″:4′, 5′]pyrido[3′,2′:4,5]thieno[3,2-d]pyrimidine-5,8-diamine
1-(3-{[5-Dimethylamino)-2,2-dimethyl-1,4-dihydro-2H-pyrano[4″, 3″:4′, 5′]pyrido[3′,2′:4,5]thieno[3,2-d]pyrimidin-8-yl]amino}propyl)pyrrolilydin-2-one
N-(2,3-Dimethoxybenzyl)-5-(pyrrolidin-1-yl)-2,2-dimethyl-1,4-dihydro-2H-pyrano[4″, 3″:4′, 5′]pyrido[3′,2′:4,5]thieno[3,2-d]pyrimidine-8-amine
2,2-Dimethyl-N-(pyridin-3-ylmethyl)-5-pyrrolidin-1-yl-1,4-dihydro-2H-pyrano[4″,3″:4′, 5′]pyrido[3′,2′:4,5]thieno[3,2-d]pyrimidin-8-amine
2,2-Dimethyl-N-(pyridin-2-ylmethyl)-5-pyrrolidin-1-yl-1,4-dihydro-2H-pyrano[4″, 3″:4′,5′]pyrido[3′,2′:4,5]thieno[3,2-d]pyrimidin-8-amine
2,2-Dimethyl-N-[2-(methylthio)benzyl]-5-pyrrolidin-1-yl-1,4-dihydro-2H-pyrano[4″,3″:4′,5′]pyrido[3′,2′:4,5]thieno[3,2-d]pyrimidin-8-amine
2,2-Dimethyl-N-[4-(methylsulfonyl)benzyl]-5-pyrrolidin-1-yl-1,4-dihydro-2H-pyrano[4″,3″:4′, 5′]pyrido[3′,2′:4,5]thieno[3,2-d]pyrimidin-8-amine
4-{[(2,2-Dimethyl-5-pyrrolidin-1-yl-1,4-dihydro-2H-pyrano[4″,3″:4′, 5′]pyrido[3′,2′:4,5]thieno[3,2-d]pyrimidin-8-yl)amino]methyl}benzenesulfonamide
1-{3-[(2,2-Dimethyl-5-pyrrolidin-1-yl-1,4-dihydro-2H-pyrano[4″,3″:4′,5′]pyrido[3′,2′:4,5]thieno[3,2-d]pyrimidin-8-yl)amino]propyl}pyrrolidin-2-one
N-[2-(1H-imidazol-4-yl)ethyl]-2,2-dimethyl-5-pyrrolidin-1-yl-1,4-dihydro-2H-pyrano[4″,3″:4′, 5′]pyrido[3′,2′:4,5]thieno[3,2-d]pyrimidin-8-amine
Ethyl 4-{2-[(2,2-dimethyl-5-pyrrolidin-1-yl-1,4-dihydro-2H-pyrano[4″, 3″:4′,5′]pyrido[3′,2′:4,5]thieno[3,2-d]pyrimidin-8-yl)amino]ethyl}piperazine-1-carboxylate
2,2-Dimethyl-N-[2-(4-methylpiperazin-1-yl)ethyl]-5-pyrrolidin-1-yl-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(quinolin-3-ylmethyl)-1,4-dihydro-2H-pyrano[4″,3″:4′,5′]pyrido[3′,2′:4,5]thieno[3,2-d]pyrimidin-8-amine
1-{3-[(2,2-Dimethyl-5-morpholin-4-yl-1,4-dihydro-2H-pyrano[4″,3″:4′, 5′]pyrido[3′,2′:4,5]thieno[3,2-d]pyrimidin-8-yl)amino]propyl}pyrrolidin-2-one
2-[(2,2-Dimethyl-5-morpholin-4-yl-1,4-dihydro-2H-pyrano[4″, 3″:4′, 5′]pyrido[3′ 2′:4,5]thieno[3,2-d]pyrimidin-8-yl)(2-morpholin-4-ylethyl)amino]ethanol
2,2-Dimethyl-5-morpholin-4-yl-N-(2-morpholin-4-ylethyl)-N-(pyridin-3-ylmethyl)-1,4-dihydro-2H-pyrano[4″,3″:4′,5′]pyrido[3′,2′:4,5]thieno[3,2-d]pyrimidin-8-amine
2,2-Dimethyl-5-morpholin-4-yl-N-(2-morpholin-4-yl-2-oxoethyl)-1,4-dihydro-2H-pyrano[4″, 3″:4′,5′]pyrido[3′,2′:4,5]thieno[3,2-d]pyrimidin-8-amine
N²-(2,2-Dimethyl-5-morpholin-4-yl-1,4-dihydro-2H-pyrano[4″, 3″:4′, 5′]pyrido[3′,2′:4,5]thieno[3,2-d]pyrimidin-8-yl)-N¹-(2-morpholin-4-ylethyl)glycinamide
2,2′-[(2,2-Dimethyl-5-morpholin-4-yl-1,4-dihydro-2H-pyrano[4″, 3″:4′,5′]pyrido[3′,2′:4,5]thieno[3,2-d]pyrimidin-8-yl)imino]diethanol
N⁵,2,2-Trimethyl-N⁸-(2-morpholin-4-ylethyl)-1,4-dihydro-2H-pyrano[4″,3″:4′,5′]pyrido[3′,2′:4,5]thieno[3,2-d]pyrimidin-5,8-diamine
N⁵,2,2-Trimethyl-N⁸-(pyridin-3-ylmethyl)-1,4-dihydro-2H-pyrano[4″,3″:4′,5′]pyrido[3′,2′:4,5]thieno[3,2-d]pyrimidin-5,8-diamine
1-[3-({5-Methylamino-2,2-dimethyl-1,4-dihydro-2H-pyrano[4″,3″:4′,5′]pyrido[3′,2′:4,5]thieno[3,2-d]pyrimidin-8-yl}amino)propyl]pyrrolidin-2-one
N⁵,2,2-Trimethyl-N⁸(2-morpholin-4-ylethyl)-N⁸-(pyridin-3-ylmethyl)-1,4-dihydro-2H-pyrano[4″, 3″:4′,5′]pyrido[3′,2′:4,5]thieno[3,2-d]pyrimidin-5,8-diamine
   and pharmaceutically acceptable salts thereof.

According to a further feature of the present invention, the compounds of formula (I) may be prepared by one of the processes described below.

Compounds Ia wherein R³ is a monosubstituted, disubstituted or unsubstituted amino group may be obtained as shown in Scheme 1.

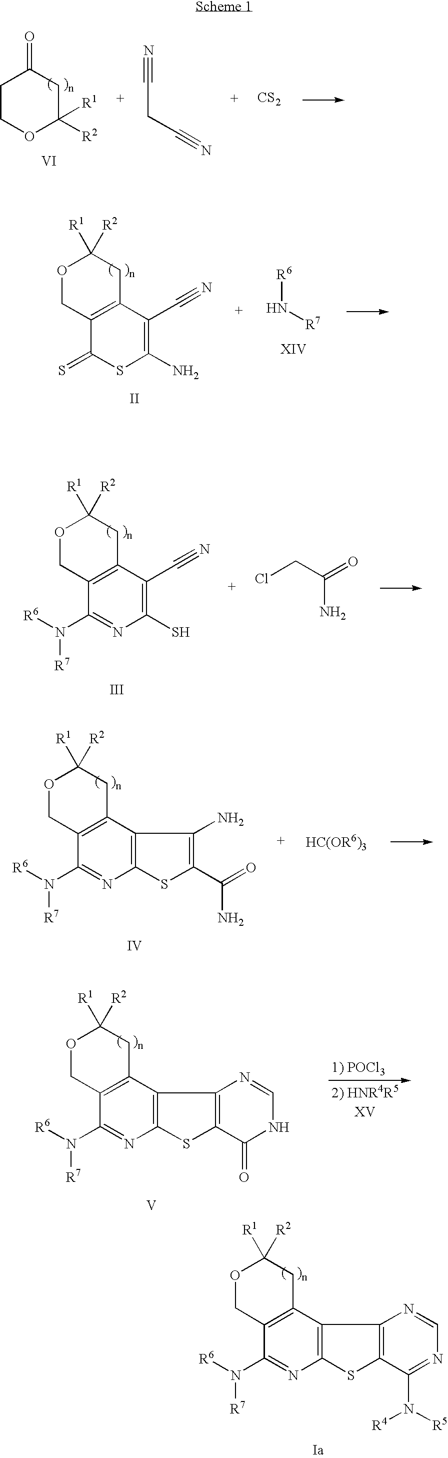

A ketone of formula VI, wherein n, R¹ and R² are as hereinbefore defined, is condensed with malononitrile in the presence of carbon disulfide to yield the heterocycle of formula II, according to the method described by E. G. Paronikyan and A. S. Noravyan at Chem. Heterocycl. Compd (NY), 1999, 35(7), 799-803. Ketones VI are commercially available or prepared according to the methods described at C. Ainsworth Org. Synth., 1959, 39, 536, J. Cologne, A. Varagnat Bull. Soc. Chim. France, 1964, 10, 2499-504, and E. M. Kosower, T. S. Sorensen, 1963, 28, 687.

Reaction of compound II with an amine HNR⁶R⁷ of formula XIV, wherein R⁶ and R⁷ are as hereinbefore defined, yields the pyridine derivative III, as described by K. Gewald et al at J. Prakt. Chem., 1973, 315(4), 679-689.

Subsequent cyclocondensation of compound III with 2-chloroacetamide in the presence of a base such as potassium carbonate affords the thienopyridine compound IV, according to C. Peinador et al J. Het. Chem., 1992, 29, 1693 or C. Peinador et al Bioorg. Med. Chem., 1998, 6,1911.

The pyridothienopyrimidine derivative V is synthesized by cyclisation of intermediate IV with a orthoformate derivative HC(OR⁶)₃, wherein R⁶ is a C₁₋₄ alkyl group, as described at C. Peinador et al Bioorg. Med. Chem., 1998, 6, 1911, or formic acid or a reactive derivative of thereof. The reactive derivative of formic acid is preferably the acid halide, orthoester or anhydride. The reaction can be carried out in a solvent, preferably a polar aprotic solvent, such as N,N-dimethylformamide, dioxane, acetone or tetrahydrofuran, in the presence of an organic base, preferably an amine base, such as triethylamine and at a temperature from 15° C. to 40° C. The reaction can also be carried out in the absence of a solvent, in which case an excess of formic acid or reactive derivative of formic acid is used and the mixture is heated at a temperature from 40° C. to its boiling point.

The corresponding chloroimine derivative of V is synthesized using phosphorous oxychloride as solvent, and the resulting intermediate is reacted with an amine of formula XV, wherein R⁴ and R⁵ are as hereinbefore defined, to give the desired final compound Ia.

When the defined groups R¹ to R⁷ are susceptible to chemical reaction under the conditions of the hereinbefore described processes or are incompatible with said processes, conventional protecting groups may be used in accordance with standard practice, for example see T. W. Greene and P. G. M. Wuts in ‘Protective Groups in Organic Chemistry’, 3^{rd} Edition, John Wiley & Sons (1999). It may be that deprotection will form the last step in the synthesis of compounds of formula I.

According to a further feature of the present invention, the pyridothienopyrimidine derivatives of general formula XIV are prepared by the process described below.

Another route for the obtention of compounds Ib is shown in Scheme 2.

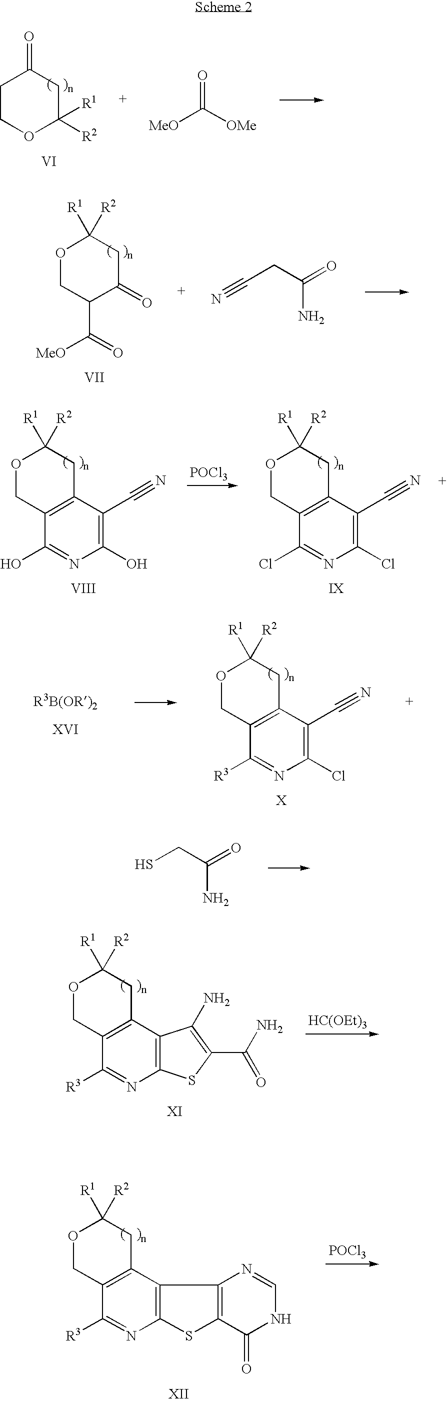

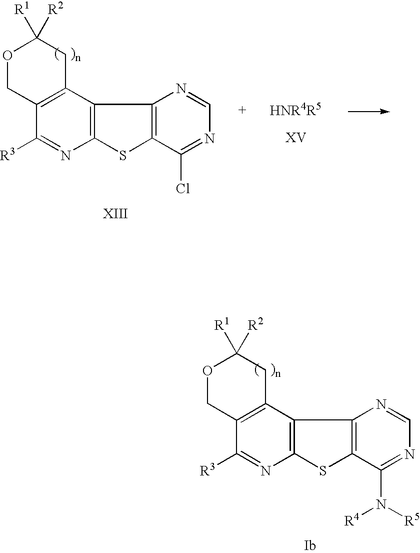

Ketone VI, wherein n, R¹ and R² are as hereinbefore defined, reacts with dimethyl carbonate in the presence of a strong base such as sodium hydride in tetrahydrofurane to yield the diketone VII, according to the method described by L. A. Paquette at J. Org. Chem., 1991, 56, 6199. Ketones VI are commercially available or may be prepared according to the methods described at C. Ainsworth Org. Synth., 1959, 39, 536, J. Cologne, A. Varagnat Bull. Soc. Chim. France, 1964, 10, 2499-504, and E. M. Kosower, T. S. Sorensen, 1963, 28, 687.

Reaction of compound VII with cyanoacetamide in methanol under refluxing conditions with the presence of potassium hydroxide yields the pyridine derivative VII, as described by E. Wenkert et al. at J. Am. Chem. Soc., 1965, 87, 5461. The same reference applies for the conversion of VII to the 1,6-dichloropyridine derivative IX by reaction with phosphorous oxychloride without solvent at 150-170° C. in a sealed tube.

IX is converted to X under classical Suzuki coupling conditions by reaction with a boronic acid of a lower alkyl boronate of formula XVI in the presence of potassium carbonate and tetrakis(triphenylphosphine)palladium(0) under reflux of dioxane, where the boronic acids R³B(OH)₂ or their corresponding boronates are commercially available or synthesized by common methodology, being R³ as hereinbefore defined.

Subsequent cyclocondensation of compound X with 2-mercaptoacetamide in the presence of a base such as potassium carbonate affords the thienopyridine compound XI, according to Santilli, A. A.; Kim, D. H.; Wanser, S. V.; J Heterocycl Chem, 1971, 8, 445 or Schneller, S. W.; Clough, F. W.; J Heterocycl Chem, 1975, 12, 513.

The pyridothienopyrimidine derivative XII is synthesized by cyclisation of intermediate XI with a orthoformate derivative HC(OEt)₃, as described at C. Peinador et al Bioorg. Med. Chem., 1998, 6, 1911, or formic acid or a reactive derivative of thereof. The reactive derivative of formic acid is preferably the acid halide, orthoester or anhydride. The reaction can be carried out in a solvent, preferably a polar aprotic solvent, such as N,N-dimethylformamide, dioxane, acetone or tetrahydrofuran, in the presence of an organic base, preferably an amine base, such as triethylamine and at a temperature from 15° C. to 40° C. The reaction can also be carried out in the absence of a solvent, in which case an excess of formic acid or reactive derivative of the carboxylic acid is used and the mixture is heated at a temperature from 40° C. to its boiling point.

The corresponding chloroimine derivative XIII is synthesized using phosphorous oxychloride as solvent, and the resulting intermediate is reacted with an amine of formula XV, wherein R⁴ and R⁵ are as hereinbefore defined, to give the desired final compound Ib.

The pharmaceutically acceptable salts of the compounds of the present invention represented by formula Ia and Ib may be acid addition salts or alkali addition salts. Examples of the acid addition salts include those formed with a mineral acid such as, for example, hydrochloric, hydrobromic, hydroiodic, sulfaric, nitric, phosphoric, or with an organic acid such as, for example, acetic, maleic, fumaric, citric, oxalic, succinic, tartaric, malic, mandelic, methanesulfonic, and p-toluenesulfonic. Examples of the alkali addition salts include inorganic salts such as, for example sodium, potassium, calcium and ammonium salts and organic alkali salts such as, for example, ethylenediamine, ethanolamine, N,N-dialkylenethanolamine, triethanolamine and basic amino acid salts.

The compounds of the present invention represented by the above described formula (Ia and Ib) may include enantiomers depending on their asymmetry or diastereoisomers. The single isomers and mixtures of the isomers fall within the scope of the present invention.

The compounds of formulae VI, XIV, XV and XVI are known compounds or can be prepared by analogy with known methods.

### Pharmacological Activity

### PDE4 Assay Procedure

Compounds to be tested were resuspended in DMSO at a stock concentration of 1 mM. The compounds were tested at different concentrations varying from 10 μM to 10 μM to calculate an IC₅₀. These dilutions were done in 96-well plates. In some cases, plates containing diluted compounds were frozen before being assayed. In these cases, the plates were thawed at room temperature and stirred for 15 minutes.

Ten microliters of the diluted compounds were poured into a “low binding” assay plate. Eighty microliters of reaction mixture containing 50 mM Tris pH 7.5, 8.3 mM MgCl₂, 1.7 mM EGTA, and 15 nM [3H]-cAMP were added to each well. The reaction was initiated by adding ten microliters of a solution containing PDE4. The plate was then incubated under stirring for 1 hour at room temperature. After incubation the reaction was stopped with 50 microlitres of SPA beads, and the reaction was allowed to incubate for another 20 minutes at room temperature before measuring radioactivity using standard instrumentation.

The reaction mixture was prepared by adding 90 ml of H₂O to 10 ml of 10× assay buffer (500 mM Tris pH 7.5, 83 mM MgCl₂, 17 mM EGTA), and 40 microlitres 1 μCi/μL [3H]-cAMP. SPA beads solution was prepared by adding 500 mg to 28 ml H₂O for a final concentration of 20 mg/ml beads and 18 mM zinc sulphate.

The results are shown in Table 1.

| Example | IC₅₀ PDE4 (nM) |
|---|---|
| 20 | 26 |
| 27 | 23 |
| 36 | 4.6 |
| 37 | 21 |
| 38 | 19 |
| 46 | 14 |
| 55 | 19 |
| 59 | 61 |
| 71 | 32 |
| 72 | 24 |
| 74 | 22 |
| 80 | 13 |

It can be seen from Table 1 that the compounds of formula (I) are potent inhibitors of phosphodiesterase 4 (PDE 4). Preferred pyridothienopyrimidine derivatives of the invention possess an IC₅₀ value for the inhibition of PDE4 (determined as defined above) of less than 100 nM, preferably less than 50 nM and most preferably less than 30 nM.

The compounds are also capable of blocking the production of some pro-inflammatory cytokines such as, for example, TNFα. Thus, they can be used in the treatment of allergic, inflammatory and immunological diseases, as well as those diseases or conditions where the blockade of pro-inflammatory cytokines or the selective inhibition of PDE 4 could be of benefit.

These disease states include asthma, chronic obstructive pulmonary disease, allergic rhinitis, rheumatoid arthritis, osteoarthritis, osteoporosis, bone-formation disorders, glomerulonephritis, multiple sclerosis, ankylosing spondylitis, Graves ophtalmopathy, myasthenia gravis, diabetes insipidus, graft rejection, gastrointestinal disorders such as ulcerative colitis or Crohn disease, septic shock, adult distress respiratory syndrome, and skin diseases such as atopic dermatitis, contact dermatitis, acute dermatomyositis and psoriasis. They can also be used as improvers of cerebrovascular function as well as in the treatment of other CNS related diseases such as dementia, Alzheimer's disease, depression, and as nootropic agents.

The compounds of the present invention are also of benefit when administered in combination with other drugs such as steroids and immunosuppressive agents, such as cyclosporin A, rapamycin or T-cell receptor blockers. In this case the administration of the compounds allows a reduction of the dosage of the other drugs, thus preventing the appearance of the undesired side effects associated with both steroids and immunosuppressants. The compounds of the invention have also shown their efficacy in blocking, after preventive and/or curative treatment, the erosive and ulcerogenic effects induced by a variety of etiological agents, such as antiinflammatory drugs (steroidal or non-steroidal antiinflammatory agents), stress, ammonia, ethanol and concentrated acids.

They can be used alone or in combination with antacids and/or antisecretory drugs in the preventive and/or curative treatment of gastrointestinal pathologies like drug-induced ulcers, peptic ulcers, H. Pylori-related ulcers, esophagitis and gastro-esophageal reflux disease. They can also be used in the treatment of pathological situations where damage to the cells or tissues is produced through conditions like anoxia or the production of an excess of free radicals. Examples of such beneficial effects are the protection of cardiac tissue after coronary artery occlusion or the prolongation of cell and tissue viability when the compounds of the invention are added to preserving solutions intended for storage of transplant organs or fluids such as blood or sperm. They are also of benefit on tissue repair and wound healing.

Accordingly, the pyridothienopyrimidine derivatives of the invention and pharmaceutically acceptable salts thereof, and pharmaceutical compositions comprising such compound and/or salts thereof, may be used in a method of treatment of disorders of the human body which comprises administering to a patient requiring such treatment an effective amount of a pyridothienopyrimidine derivative of the invention or a pharmaceutically acceptable salt thereof.

The present invention also provides pharmaceutical compositions which comprise, as an active ingredient, at least a pyridothienopyrimidine derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight, of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Compositions for topical administration may take the form of ointments, creams or lotions, all containing the compound of the invention; such preparations may be made by methods well-known in the art.

Effective doses are normally in the range of 10-600 mg of active ingredient per day. Daily dosage may be administered in one or more treatments, preferably from 1 to 4 treatments, per day.

The syntheses of the compounds of the invention and of the intermediates for use therein are illustrated by the following Examples (including Preparation Examples (Preparations 1 to 63)) which do not limit the scope of the invention in any way.

¹H Nuclear Magnetic Resonance Spectra were recorded on a Varian Gemini 300 spectrometer.

Low Resolution Mass Spectra (m/z) were recorded on a Micromass ZMD mass spectrometer using ESI ionization.

Melting points were recorded using a Perkin Elmer DSC-7 apparatus.

The chromatographic separations were obtained using a Waters 2690 system equipped with a Symmetry C18 (2.1×10 mm, 3.5 mM) column. The mobile phase was formic acid (0.4 mL), ammonia (0.1 mL), methanol (500 mL) and acetonitrile (500 mL) (B) and formic acid (0.46 mL), ammonia (0.115 mL) and water (1000 mL) (A): initially from 0% to 95% of B in 20 min, and then 4 min. with 95% of B. The reequilibration time between two injections was 5 min. The flow rate was 0.4 mL/min. The injection volume was 5 microliter. Diode array chromatograms were collected at 210 nM.

### PREPARATION EXAMPLES

### Preparation 1

### 1-Hydroxy-5-methyl-hexa-1,4-dien-3-one

To a suspension of sodium hydride (2.04 g, 50.9 mmol) in ethyl ether (100 ml) ethanol (0.25 ml) was added in one portion. Once this suspension is cooled in an ice-bath, a mixture of mesityl oxide (5.0 g, 50.9 mmol) and ethyl formate (6.17 ml, 76.4 mmol) in ethyl ether (20 ml) is dropwise added. This final mixture is stirred at 0° C. for 6 h and then allowed to reach room temperature overnight. Ethanol (1 ml) is then added and the reaction mixture is stirred at room temperature for one hour. Water (10 ml) is added in one portion and two phases are separated. The organic phase is washed twice with water. These aqueous phases are put together and washed with ethyl ether, then acidified with 6N chlorhidric acid (8.25 ml) and finally extracted repeatedly with ethyl ether. The collected organic phases are washed with brine, dried over magnesium sulfate, filtered and the solvent evaporated under vacuum. 5.10 g of the desired compound is obtained as an orange oil, pure enough to perform the next synthetic step. Yield=79%.

¹H NMR (200 MHz, CDCl₃) δ ppm 1.9 (s, 3H), 2.2 (s, 3H), 3.5 (m. 1H), 5.4 (d, 1H), 5.8 (d, 1H), 8.2 (d, 1H).

### Preparation 2

### 2,2-Dimethyl-2,3-dihydropyran-4-one

A suspension of 1-hydroxy-5-methyl-hexa-1,4-dien-3-one (0.5 g, 3.96 mmol, see Preparation 1), mercurium sulphate (0.05 g, 0.17 mmol) and 10% sulfuric acid (5 ml) is heated at 100° C. for 3 h. The resultant mixture is poured over an ice bath and basified with 2N NaOH to pH=11. After extraction with ethyl ether, the organic phase is washed with brine, dried over magnesium sulfate, filtered and the solvent evaporated under vacuum to yield 0.2 g of the desired final product. Further extraction with ethyl ether of the acidified aquous phase yields 0.3 g more of final product. Yield=60%.

¹H NMR (200 MHz, CDCl₃) δ ppm 1.45 (s, 6H), 2.5 (s, 2H), 5.4 (d, 2H), 7.2 (d, 2H).

### Preparation 3

### 2,2-Dimethyltetrahydropyran-4-one

The resulting compound of preparation 2 (0.5 g, 3.96 mmol) is hydrogenated at 30 psi in a Parr apparatus using 10% Pd over charcoal (0.05 g) as catalyst and a mixture of ethyl acetate (10 ml) and acetic acid (0.5 ml) as solvent until the reaction is completed. The catalyst is then filtered and the liquid phase is washed with sodium bicarbonate, water and brine, dried over magnesium sulfate, filtered and the solvent evaporated under vacuum, to yield 0.35 g of the desired final compound as a yellowish oil. Yield=69%.

¹H NMR (200 MHz, CDCl₃) δ ppm 1.3 (s, 6H), 2.4 (s, 2H), 2.45 (t, 2H), 4.05 (t, 2H).

### Preparation 4

### 6-Amino-3,3-dimethyl-8-thioxo-4,8-dihydro-1H,3H-thiopyrano[3,4-c]pyran-5-carbonitrile

2,2-Dimethyltetrahydropyran-4-one (5.0 g, 32.0 mmol, see Preparation 3) is solved in methanol (4.7 ml) and carbon disulfide (4.7 ml, 48.8 mmol) is added in one portion. Malononitrile (2.6 g, 39.0 mmol) is added portionwise and, finally, triethylamine (1.95 ml). The reaction mixture is stirred at room temperature for 48 h. An orange precipitate is formed, which is filtered (3.90 g) and is consistent with the desired compound. From the liquid phase, 0.89 g more of 6-amino-3,3-dimethyl-8-thioxo-4,8-dihydro-1H,3H-thiopyrano[3,4-c]pyran-5-carbonitrile were isolated by flash chromatography, eluting first with CH₂Cl₂ and next with the mixture of solvents CH₂Cl₂:MeOH 98:2. Yield=48%.

¹H NMR (200 MHz, CDCl₃) δ ppm 1.30 (s, 6H), 2.62 (s, 2H), 4.66 (s, 2H), 7.91 (s, 2H)

### Preparation 5

### 6-Mercapto-3,3-dimethyl-8-morpholin-4-yl-3,4-dihydro-1H-pyrano[3,4-c]pyridine-5-carbonitrile

The product resulting from preparation 4 (3.9 g, 15.45 mmol) is suspended in ethanol (17 ml) and morpholine (6.7 ml, 77.3 mmol) is added. The reaction mixture is refluxed under nitrogen overnight. Then the system is allowed to reach room temperature and the reaction mixture is left in an ice bath for two hours. The solid formed is filtrated and washed twice with ethanol. After drying, 3.12 g of the final compound are obtained as a dark solid, pure enough to perform the next step. Yield=66%.

¹H NMR (200 MHz, CDCl₃) δ ppm 1.30 (s, 6H), 2.75 (s, 2H), 3.3 (m, 4H), 3.75 (m, 4H), 4.5 (s, 2H).

### Preparation 6

### 1-Amino-8,8-dimethyl-5-morpholin-4-yl-8,9-dihydro-6H-pyrano[4,3-d]thieno[2,3-b]pyridine-2-carboxamide

To a suspension of 6-mercapto-3,3-dimethyl-8-morpholin-4-yl-3,4-dihydro-1H-pyrano[3,4-c]pyridine-5-carbonitrile (3.12 g, 10.22 mmol, see Preparation 5) in ethanol (150 ml), potassium carbonate (3.3 g, 24.5 mmol) and 2-chloroacetamide (1.05 g, 11.24 mmol) are added, and the reaction mixture is then refluxed for 4 h. The solvent is evaporated under vacuum and water is added to the residue: the precipitated solid is filtered and dried. It weighs 3.0 g and its ¹H-RMN is consistent with the desired product. Yield=81%.

¹H NMR (200 MHz, DMSO-D6) δ ppm 1.29 (s, 6H) 3.08 (m, 4H) 3.20 (s, 2H) 3.73 (m, 4H) 4.64 (s, 2H) 6.81 (s, 2H) 7.07 (s, 2H)

### Preparation 7

### 2,2-Dimethyl-5-morpholin-4-yl-1,4-dihydro-2H-pyrano[4″,3″:4′,5′]pyrido[3′,2′:4,5]thieno[3,2-d]pyrimidin-8(9H)-one

1-Amino-8,8-dimethyl-5-morpholin-4-yl-8,9-dihydro-6H-pyrano[4,3-d]thieno[2,3-b]pyridine-2-carboxamide (3.0 g, 8.3 mmol, see Preparation 6) is suspended in ethyl orthoformate (50 ml) and p-toluensulfonic acid hydrate (0.16 g, 0.83 mmol) is added. This mixture is heated under reflux overnight. Then the reaction mixture is allowed to reach room temperature and left in an ice bath for two hours. The precipitated formed is filtered and washed with ethyl ether. After drying it weighs 2.8 g and its ¹H-RMN is consistent with the desired compound. Yield=92%.

¹H NMR (200 MHz, DMSO-D6) δ ppm 1.32 (s, 6H) 3.20 (m, 4H) 3.44 (s, 2H) 3.76 (m, 4H) 4.70 (s, 2H) 8.33 (s, 1H).

### Preparation 8

### 8-Chloro-2,2-dimethyl-5-morpholin-4-yl-1,4-dihydro-2H-pyrano[4″,3″:4′,5′]pyrido[3′,2′:4,5]thieno[3,2-d]pyrimidine

The final product of preparation 7 (2.84 g, 7.63 mmol) is suspended in phosphorous oxychloride (30 ml) and heated to reflux for 90 min. The excess of phosphorous oxychloride is evaporated under vacuum and the residue redissolved between chloroform and a cooled solution of 2N NaOH. The aqueous phase is extracted twice with chloroform and the organic phases are washed with water and brine, dried over magnesium sulfate, filtered and the solvent evaporated. 2.98 g of a brownish solid is obtained, which ¹H-RMN is consistent with the desired compound. Yield=100%.

¹H NMR (200 MHz, CHLOROFORM-D) δ ppm 1.44 (s, 6H) 3.35 (m, 4H) 3.57 (s, 2H) 3.88 (m, 4H) 4.78 (s, 2H) 9.02 (s, 1H)

### Preparation 9

### 6-Mercapto-8-[(2-methoxyethyl)methylamino]-3,3-dimethyl-3,4-dihydro-1H-pyrano[3,4-c]pyridine-5-carbonitrile

The product resulting from preparation 4 (2.19 g, 8.68 mmol) is suspended in a mixture of ethanol (15 ml) and dimethylformamide (5 ml), and (2-methoxyethyl)(methyl)amine (4.41 g, 49.5 mmol) is added. The reaction mixture is heated at 100° C. under nitrogen for 4 h and left overnight at room temperature. The solvents are evaporated under vacuum and the resulting residue is purificated by flash chromatography, eluting with the mixture CH₂Cl₂:MeOH 9:1. 2.02 g of the final product as an oil is obtained. Yield=76%.

¹H NMR (300 MHz, CHLOROFORM-D) δ ppm 1.35 (s, 6H) 1.6 (s, 1H) 2.85 (m, 2H) 2.90 (s, 3H) 2.95 (s, 3H), 3.36 (m, 2H) 3.64 (d, J=9.07 Hz, 2H) 4.67 (m, 2H)

### Preparation 10

### 5-[(2-Methoxyethyl)(methyl)amino]-1,8,8-trimethyl-8,9-dihydro-6H-pyrano[4,3-d]thieno[2,3-b]pyridine-2-carboxamide

To a suspension of 6-mercapto-8-[(2-methoxyethyl)methylamino]-3,3-dimethyl-3,4-dihydro-1H-pyrano[3,4-c]pyridine-5-carbonitrile (2.00 g, 6.51 mmol, see Preparation 15) in ethanol (100 ml), potassium carbonate (2.16 g, 15.6 mmol) and 2-chloroacetamide (0.67 g, 7.16 mmol) are added, and the reaction mixture is then refluxed under nitrogen overnight. The solvent is evaporated under vacuum and water is added to the residue. After successive extractions with chloroform, the organic phase is dried over magnesium sulfate, filtered and the solvent is evaporated. The final product (0.34 g) is isolated by flash chromatography, eluting with CH₂Cl₂:MeOH 98:2. Yield=15%.

¹H NMR (300 MHz, CHLOROFORM-D) δ ppm 1.39 (s, 6H) 1.60 (s, 2H) 2.95 (s, 2H) 3.13 (s, 2H) 3.34 (s, 3H) 3.41 (t, J=5.91 Hz, 2H) 3.59 (t, J=6.04 Hz, 2H) 4.74 (s, 2H) 5.26 (m, 1H) 6.34 (s, 2H).

### Preparation 11

### 5-[2-Methoxyethyl)(methyl)amino]-2,2-dimethyl-1,4-dihydro-2H-pyrano[4″,3″:4′,5′]pyrido[3′,2′:4,5]thieno[3,2-a]pyrimidin-8(9M-one

5-[(2-Methoxyethyl)(methyl)amino]-1,8,8-trimethyl-8,9-dihydro-6H-pyrano[4,3-d]thieno[2,3-b]pyridine-2-carboxamide (0.34 g, 0.94 mmol, see Preparation 16) is suspended in ethyl orthoformate (7 ml) and p-toluensulfonic acid hydrate (0.02 g, 0.09 mmol) is added. This mixture is heated under reflux overnight. Then the reaction mixture is allowed to reach room temperature and left in an ice bath for two hours. The precipitated formed is filtered and washed with ethyl ether. After drying it weighs 0.13 g and its ¹H-RMN is consistent with the desired compound. Additional 0.14 g of the desired final product are isolated by column chromatography from the non-precipitated residue, eluting first with CH₂Cl₂:MeOH 98:2 and then with CH₂Cl₂:MeOH 95:5. Yield=76%.

¹H NMR (300 MHz, CHLOROFORM-D) δ ppm 1.40 (s, 6H) 1.60 (s, 2H) 3.05 (s, 3H) 3.35 (s, 3H) 3.50 (m, 4H) 3.70 (m, 2H) 4.80 (s, 2H) 8.15 (s, 1H) 12.4 (s, 1H).

### Preparation 12

### 8-Chloro-N-(2-methoxyethyl)-N,2,2-trimethyl-1,4-dihydro-2H-pyrano[4″,3″:4′,5′]pyrido[3′,2′:4,5]thieno[3,2-d]pyrimidine-5-amine.

The final product of preparation 17 (0.27 g, 0.72 mmol) is suspended in phosphorous oxychloride (7 ml) and heated to reflux for 90 min. The excess of phosphorous oxychloride is evaporated under vacuum and the residue redissolved between chloroform and a cooled solution of 2N NaOH. The aqueous phase is extracted twice with chloroform and the organic phases are washed with water and brine, dried over magnesium sulfate, filtered and the solvent evaporated. 0.29 g of a brownish solid is obtained, which ¹H-RMN is consistent with the desired compound. Yield=100%.

¹H NMR (300 MHz, CHLOROFORM-D) δ ppm 1.41 (s, 6H) 3.05 (s, 3H) 3.35 (s, 3H) 3.45 (s, 2H) 3.60-3.79 (m, 4H) 4.80 (s, 2H) 9.0 (s, 1H).

### Preparation 13

### 6-Mercapto-3,3-dimethyl-8-(4-methylpiperazin-1-yl)-3,4-dihydro-1H-pyrano[3,4-c]pyridine-5-carbonitrile

The product resulting from preparation 4 (1.50 g, 5.94 mmol) is suspended in ethanol (10 ml) and N-methylpiperazine (3.76 ml, 33.9 mmol) is added. The reaction mixture is heated at 100° C. under nitrogen overnight. The solvent is evaporated under vacuum and the resulting residue is pure enough to perform the next synthetic step.

¹H NMR (300 MHz, CHLOROFORM-D) δ ppm 1.35 (s, 6H) 2.30 (s, 3H) 2.40 (s, 1H) 2.50 (m, 4H), 2.70 (s, 2H) 3.00 (m, 4H) 4.67 (m, 2H)

### Preparation 14

### 1-Amino-8,8-dimethyl-5-(4-methylpiperazin-1-yl)-8,9-dihydro-6H-pyrano[4,3-d]thieno[2,3-b]pyridine-2-carboxamide

To a suspension of 6-mercapto-3,3-dimethyl-8-(4-methylpiperazin-1-yl)-3,4-dihydro-1H-pyrano[3,4-c]pyridine-5-carbonitrile (1.89 g, 5.94 mmol, see Preparation 19) in ethanol (100 ml), potassium carbonate (1.72 g, 12.5 mmol) and 2-chloroacetamide (0.61 g, 6.53 mmol) are added, and the reaction mixture is then refluxed under nitrogen for 6 h and then left at room temperature overnight. The solvent is evaporated under vacuum and water is added to the residue. A solid precipitates, which is filtered and washed with water. Once dried, it weighs 1.08 g. The NMR is consistent with the final product. Yield=48%.

¹H NMR (200 MHz, CHLOROFORM-D) δ ppm 1.38 (s, 6H) 2.36 (s, 3H) 2.58 (s, 4H) 3.18 (s, 6H) 4.71 (s, 2H) 5.90 (s, 2H) 6.42 (s, 2H)

### Preparation 15

### 2,2-Dimethyl-5-(4-methylpiperazin-1-yl)-1,4-dihydro-2H-pyrano[4″,3″:4′,5′]pyrido[3′,2′:4,5]thieno[3,2-d]pyrimidin-8(9H)-one.

1-Amino-8,8-dimethyl-5-(4-methylpiperazin-1-yl)-8,9-dihydro-6H-pyrano[4,3-d]thieno[2,3-b]pyridine-2-carboxamide (1.08 g, 2.87 mmol, see Preparation 20) is suspended in ethyl orthoformate (20 ml) and p-toluensulfonic acid hydrate (0.06 g, 0.29 mmol) is added. This mixture is heated under reflux for 2 h. Then the reaction mixture is allowed to reach room temperature and left in an ice bath for two hours. The precipitated formed is filtered and washed with ethyl ether. After drying it weighs 1.02 g and its ¹H-NMR is consistent with the desired compound. Yield=93%

¹H NMR (200 MHz, CHLOROFORM-D) δ ppm 1.41 (s, 6H) 2.43 (s, 3H) 2.67 (m, 4H) 3.34 (m, 4H) 3.50 (s, 2H) 4.76 (s, 2H) 8.08 (s, 1H)

### Preparation 16

### 8-Chloro-2,2-dimethyl-5-(4-methylpiperazin-1-yl)-1,4-dihydro-2H-pyrano[4″,3″:4′,5′]pyrido[3′,2′:4,5]thieno[3,2-a]pyrimidine.

The final product of preparation 21 (1.02 g, 2.65 mmol) is suspended in phosphorous oxychloride (20 ml) and heated to reflux for 90 min. The excess of phosphorous oxychloride is evaporated under vacuum and the residue redissolved between chloroform and a cooled solution of 2N NaOH. The aqueous phase is extracted twice with chloroform and the organic phases are washed with water and brine, dried over magnesium sulfate, filtered and the solvent evaporated. 0.86 g of a brownish solid is obtained, which ¹H-RMN is consistent with the desired compound. Yield=80%.

¹H NMR (200 MHz, CHLOROFORM-D) δ ppm 1.44 (s, 6H) 2.39 (s, 3H) 2.61 (m, 4H) 3.40 (m, 4H) 3.57 (s, 2H) 4.77 (s, 2H) 9.00 (s, 1H)

### Preparation 17

### 6-Mercapto-3,3-dimethyl-8-(piperidin-1-yl)-3,4-dihydro-1H-pyrano[3,4-c]pyridine-5-carbonitrile

The product resulting from preparation 4 (3.0 g, 11.9 mmol) is suspended in ethanol (13.5 ml) and piperidine (6.71 ml, 67.8 mmol) is added. The reaction mixture is heated at 100° C. under nitrogen for 4 h. The solvent is evaporated under vacuum and the resulting residue is pure enough to perform the next synthetic step.

¹H NMR (300 MHz, CHLOROFORM-D) δ ppm 1.30 (s, 6H) 1.70 (m, 7H) 2.70 (s, 2H) 3.70 (m, 4H) 4.80 (s, 2H)

### Preparation 18

### 1-Amino-8,8-dimethyl-5-(piperidin-1-yl)-8,9-dihydro-6H-pyrano[4,3-d]thieno[2,3-b]pyridine-2-carboxamide

To a suspension of 6-mercapto-3,3-dimethyl-8-(piperidin-1-yl)-3,4-dihydro-1H-pyrano[3,4-c]pyridine-5-carbonitrile (3.61 g, 11.9 mmol, see Preparation 23) in ethanol (180 ml), potassium carbonate (3.94 g, 28.6 mmol) and 2-chloroacetamide (1.22 g, 13.1 mmol) are added, and the reaction mixture is then refluxed under nitrogen for 4 h and then left at room temperature overnight. The solvent is evaporated under vacuum and water is added to the residue. A solid precipitates, which is filtered and washed with water. Once dried, it weighs 2.76 g. The MS is consistent with the final product. Yield=64%.

LRMS: m/z 361 (M+1)⁺

### Preparation 19

### 2,2-Dimethyl-5-(piperidin-1-yl)-1,4-dihydro-2H-pyrano[4″,3″:4′,5′]pyrido[3′,2′:4,5]thieno[3,2-d]pyrimidin-8(9H)-one.

1-Amino-8,8-dimethyl-5-(piperidin-1-yl)-8,9-dihydro-6H-pyrano[4,3-d]thieno[2,3-b]pyridine-2-carboxamide (0.70 g, 1.94 mmol, see Preparation 24) is suspended in ethyl orthoformate (15 ml) and p-toluensulfonic acid hydrate (0.04 g, 0.19 mmol) is added. This mixture is heated under reflux for 3 h. Then the reaction mixture is allowed to reach room temperature and left in an ice bath for two hours. The precipitated formed is filtered and washed with ethyl ether. After drying it weighs 0.48 g and its ¹H-NMR is consistent with the desired compound. Yield=66%

¹H NMR (300 MHz, CHLOROFORM-D) δ ppm 1.40 (s, 6H) 1.70 (m, 6H) 3.25 (m, 4H) 3.50 (s, 2H) 4.80 (s, 2H) 8.25 (s, 1H) 12.5 (s, 1H)

### Preparation 20

### 8-Chloro-2,2-dimethyl-5-(piperidin-1-yl)-1,4-dihydro-2H-pyrano[4″,3″:4′,5′]pyrido[3′,2′:4,5]thieno[3,2-a]pyrimidine.

The final product of preparation 19 (0.48 g, 1.28 mmol) is suspended in phosphorous oxychloride (10 ml) and heated to reflux for 90 min. The excess of phosphorous oxychloride is evaporated under vacuum and the residue redissolved between chloroform and a cooled solution of 2N NaOH. The aqueous phase is extracted twice with chloroform and the organic phases are washed with NaOH 2N, water and brine, dried over magnesium sulfate, filtered and the solvent evaporated. 0.49 g of a violet solid is obtained, which ¹H-RMN is consistent with the desired compound. Yield=98%.

¹H NMR (200 MHz, CHLOROFORM-D) δ ppm 1.40 (s, 6H) 1.70 (m, 6H) 3.35 (m, 4H) 3.57 (s, 2H) 4.77 (s, 2H) 9.00 (s, 1H)

### Preparation 21

### 6-Mercapto-3,3-dimethyl-8-(pyrrolidin-1-yl)-3,4-dihydro-1H-pyrano[3,4-c]pyridine-5-carbonitrile

The product resulting from preparation 4 (1.97 g, 7.81 mmol) is suspended in ethanol (14 ml) and pyrrolidine (3.71 ml, 44.5 mmol) is added. The reaction mixture is heated at 100° C. under nitrogen for 3 h. The solvent is evaporated under vacuum and the resulting residue is purified by column chromatography eluting with a mixture of CH₂Cl₂:MeOH 98:2. 1.27 g of the final compound is obtained as an orange solid. Yield=56%

¹H NMR (300 MHz, CHLOROFORM-D) δ ppm 1.30 (s, 6H) 1.70 (m, 1H) 2.10 (m, 4H) 2.70 (s, 2H) 3.70 (m, 4H) 4.80 (s, 2H)

### Preparation 22

### 1-Amino-8,8-dimethyl-5-(pyrrolidin-1-yl)-8,9-dihydro-6H-pyrano[4,3-d]thieno[2,3-b]pyridine-2-carboxamide

To a suspension of 6-mercapto-3,3-dimethyl-8-(pyrrolidin-1-yl)-3,4-dihydro-1H-pyrano[3,4-c]pyridine-5-carbonitrile (1.27 g, 4.39 mmol, see Preparation 27) in ethanol (65 ml), potassium carbonate (1.27 g, 9.21 mmol) and 2-chloroacetamide (0.45 g, 4.83 mmol) are added, and the reaction mixture is then refluxed under nitrogen for 6 h and then left at room temperature overnight. The solvent is evaporated under vacuum and water is added to the residue. A solid precipitate, which is filtered and washed with water. Once dried, it weighs 1.25 g. The NMR is consistent with the final product. Yield=82%.

¹H NMR (300 MHz, CHLOROFORM-D) δ ppm 1.60 (s, 6H) 1.95 (m, 4H) 3.10 (s, 2H) 3.55 (m, 4H) 4.80 (s, 2H) 5.20 (s, 2H) 6.35 (s, 2H)

### Preparation 23

### 2,2-Dimethyl-5-(pyrrolidin-1-yl)-1,4-dihydro-2H-pyrano[4″,3″:4′,5′]pyrido[3′,2′:4,5]thieno[3,2-d]pyrimidin-8(9H)-one.

1-Amino-8,8-dimethyl-5-(pyrrolidin-1-yl)-8,9-dihydro-6H-pyrano[4,3-d]thieno[2,3-b]pyridine-2-carboxamide (1.25 g, 3.61 mmol, see Preparation 28) is suspended in ethyl orthoformate (25 ml) and p-toluensulfonic acid hydrate (0.07 g, 0.36 mmol) is added. This mixture is heated under reflux for 15 h. Then the reaction mixture is allowed to reach room temperature and left in an ice bath for two hours. The precipitated formed is filtered and washed with ethyl ether. After drying it weighs 0.23 g and its ¹H-NMR is consistent with the desired compound. Another batch of final product (0.85 g) is obtained by flash chromatography (eluting with CH₂Cl₂:MeOH 98:2) of the residue obtained after evaporation of the solvent. Global yield=83%

¹H NMR (300 MHz, CHLOROFORM-D) δ ppm 1.40 (s, 6H) 1.95 (m, 4H) 3.45 (s, 2H) 3.65 (m, 4H) 4.95 (s, 2H) 8.25 (s, 1H) 12.3 (s, 1H)

### Preparation 24

### 8-Chloro-2,2-dimethyl-5-(pyrrolidin-1-yl)-1,4-dihydro-2H-pyrano[4″,3″:4′,5′]pyrido[3′,2′:4,5]thieno[3,2-d]pyrimidine.

The final product of preparation 29 (1.08 g, 3.02 mmol) is suspended in phosphorous oxychloride (20 ml) and heated to reflux for 90 min. The excess of phosphorous oxychloride is evaporated under vacuum and the residue redissolved between chloroform and a cooled solution of 2N NaOH. The aqueous phase is extracted twice with chloroform and the organic phases are washed with water and brine, dried over magnesium sulfate, filtered and the solvent evaporated. 1.17 g of a solid is obtained, which ¹H-RMN is consistent with the desired compound. Quantitative yield.

¹H NMR (200 MHz, CHLOROFORM-D) δ ppm 1.40 (s, 6H) 2.00 (m, 4H) 3.45 (s, 2H) 3.70 (m, 4H) 4.95 (s, 2H) 8.95 (s, 1H)

### Preparation 25

### 8-Chloro-2-ethyl-2-methyl-5-morpholin-4-yl-1,4-dihydro-2H-pyrano[4″,3″:4′,5′]pyrido[3′,2′:4,5]thieno[3,2-d]pyrimidine

2-Ethyl-2-methyl-5-morpholin-4-yl-1,4-dihydro-2H-pyrano[4″,3″:4′,5′]pyrido[3′,2′:4,5]thieno[3,2-d]pyrimidin-8(9H)-one (0.50 g, 1.28 mmol, commercially available at Pharmeks Ltd., ref.nr.PHAR024687) is suspended in phosphorous oxychloride (10 ml) and the mixture is refluxed for 90 minutes. The excess of POCl₃ is evaporated under vacuum and the residue is redissolved between NaOH 2N and chloroform. The aqueous phase is extracted twice with chloroform. The organic phase is washed with water and brine, dried over magnesium sulfate, filtered and evaporated. 0.52 g of a greenish oil is obtained, pure enough to perform the following synthetic step. Quantitative yield.

¹H NMR (200 MHz, CHLOROFORM-D) δ ppm 1.00 (t, 3H) 1.35 (s, 3H) 1.70 (m, 2H) 3.35 (m, 4H) 3.55 (s, 2H) 3.90 (m, 4H) 4.75 (s, 2H) 9.05 (s, 1H)

### Preparation 26

### 6-Amino-8-thioxo-4,8-dihydro-1H,3H-thiopyrano[3,4-c]pyran-5-carbonitrile

Tetrahydropyran-4-one (5.00 g, 50.0 mmol) is dissolved in methanol (6 ml) and carbon disulfide (6.00 ml, 10.0 mmol), malonodinitrile (3.30 g, 50.0 mmol, in portions) and, finally, triethylamine (2.50 ml, 136.0 mmol, dropwise) are carefully added in this order (CAUTION! During the addition of the base, a vigorous exothermic reaction takes place, with concomitant precipitation of a white solid). This mixture is stirred for 24 h. The precipitated solid is filtered, washed with cold methanol and recristalized from 2-propanol. 6.27 g of final product as a red solid are obtained. Yield=56%

¹H NMR (300 MHz, CHLOROFORM-D) δ ppm 2.75 (t, 2H) 3.90 (t, 2H) 4.65 (s, 2H) 7.80 (bs, 2H)

### Preparation 27

### 6-Mercapto-8-(morpholin-1-yl)-3,4-dihydro-1H-pyrano[3,4-c]pyridine-5-carbonitrile

6-Amino-8-thioxo-4,8-dihydro-1H,3H-thiopyrano[3,4-c]pyran-5-carbonitrile (1.00 g, 4.46 mmol, see Preparation 55) is dissolved in ethanol (4.5 ml) and morpholine (2.25 ml, 25.82 mmol) is added. After refluxing under nitrogen for 4 h, the mixture is allowed to reach room temperature. 0.76 g of the final compound are obtained by filtration. Further 0.30 g of this product are isolated by acidification with acetic acid of the concentrated organic phase diluted with water. Both solids are put together and recristalized from methanol. 1.02 g of the desired product are obtained. Yield=82%

¹H NMR (300 MHz, CHLOROFORM-D) δ ppm 2.90 (m, 2H) 3.25 (m, 4H) 3.65 (m, 1H) 3.80 (m, 4H) 4.00 (m, 2H) 4.45 (s, 2H)

### Preparation 28

### 1-Amino-5-(morpholin-1-yl)-8,9-dihydro-6H-pyrano[4,3-d]thieno[2,3-b]pyridine-2-carboxamide

6-Mercapto-8-(morpholin-1-yl)-3,4-dihydro-1H-pyrano[3,4-c]pyridine-5-carbonitrile (0.30 g, 1.08 mmol, see Preparation 56) is suspended in ethanol (15 ml) and potassium carbonate (0.34 g, 2.42 mmol) and 2-chloroacetamide (0.11 g, 1.19 mmol) are added. This mixture is refluxed for 3 h. The solvent is evaporated and the residue redissolved in ethyl acetate and water saturated with potassium carbonate. After extraction with ethyl acetate, the organic phase is dried over magnesium sulfate, filtered and evaporated. 0.16 g of the desired final compound are obtained, pure enough to perform the next synthetic step. Yield=44%

¹H NMR (300 MHz, CHLOROFORM-D) δ ppm 3.20 (m, 4H) 3.35 (t, 2H) 3.85 (m, 4H) 4.10 (t, 2H) 4.70 (s, 2H) 5.70 (s, 2H) 6.40 (s, 2H)

### Preparation 29

### 5-(Morpholin-1-yl)-1,4-dihydro-2H-pyrano[4″,3″:4′,5′]pyrido[3′,2′:4,5]thieno[3,2-d]pyrimidin-8(9H)-one

1-Amino-5-(morpholin-1-yl)-8,9-dihydro-6H-pyrano[4,3-d]thieno[2,3-b]pyridine-2-carboxamide (0.16 g, 0.47 mmol, see Preparation 57) is suspended is ethyl orthoformate (5 ml) and acid p-toluensulfonic monohydrate (0.01 g, 0.05 mmol) is added. This mixture is refluxed overnight. Once at room temperature, a solid precipitates. After leaving the mixture in an ice bath, 0.07 g of the final product is isolated by filtration and subsequent drying. Yield=42%.

¹H NMR (300 MHz, CHLOROFORM-D) δ ppm 3.25 (m, 4H) 3.65 (t, 2H) 3.85 (m, 4H) 4.10 (t, 2H) 4.75 (s, 2H) 8.10 (s, 1H) 12.45 (bs, 1H)

### Preparation 30

### 8-Chloro-5-(morpholin-1-yl)-1,4-dihydro-2H-pyrano[4″,3″:4′,5′]pyrido[3′,2′:4,5]thieno[3,2-d]pyrimidine

5-(Morpholin-1-yl)-1,4-dihydro-2H-pyrano[4″,3″:4′,5′]pyrido[3′,2′:4,5]thieno[3,2-d]pyrimidin-8(9H)-one (0.07 g, 1.98 mmol, see Preparation 58) is suspended in phosphorous oxychloride (3 ml) and this mixture is refluxed for 90 minutes. The solvent is evaporated under vacuum. To the residue ice is added and then NaOH 2N dropwise until the pH becomes basic. This aqueous phase is extracted repeatedly with chloroform. The organic phase is washed with brine, dried over magnesium sulfate, filtered and evaporated. 0.05 g of a brownish solid is obtained, whose ¹HNMR is consistent with the proposed final structure. Yield=70%

¹H NMR (300 MHz, CHLOROFORM-D) δ ppm 3.30 (m, 4H) 3.65 (t, 2H) 3.85 (m, 4H) 4.15 (t, 2H) 4.75 (s, 2H) 9.0 (s, 1H)

### Preparation 31

### 6-Mercapto-8-(pyrrolidin-1-yl)-3,4-dihydro-1H-pyrano[3,4-c]pyridine-5-carbonitrile

6-Amino-8-thioxo-4,8-dihydro-1H,3H-thiopyrano[3,4-c]pyran-5-carbonitrile (2.50 g, 11.15 mmol, see Preparation 55) is dissolved in ethanol (11.25 ml) and pyrrolidine (5.30 ml, 63.5 mmol) is added. After refluxing under nitrogen for 16 h, the mixture is allowed to reach room temperature. The solvent is evaporated under vacuum and the residue is purified by flash chromatography, eluting with CH₂Cl₂:MeOH 95:5. 1.10 g of the final compound is isolated. Yield=38%.

LRMS: m/z 262 (M+1)+

### Preparation 32

### 1-Amino-5-(pyrrolidin-1-yl)-8,9-dihydro-6H-pyrano[4,3-b]thieno[2,3-b]pyridine-2-carboxamide

6-Mercapto-8-(pyrrolidin-1-yl)-3,4-dihydro-1H-pyrano[3,4-c]pyridine-5-carbonitrile (1.10 g, 4.21 mmol, see Preparation 60) is suspended in ethanol (60 ml) and potassium carbonate (1.40 g, 10.10 mmol) and 2-chloroacetamide (0.43 g, 4.63 mmol) are added. This mixture is refluxed for 3 h. The solvent is evaporated and the residue is treated with water. An insoluble solid is filtered and dried. 0.88 g of a brown solid is obtained, whose ¹HNMR is consistent with the final product. Yield=66%.

¹H NMR (300 MHz, CHLOROFORM-D) δ ppm 1.95 (m, 4H) 2.30 (t, 2H) 3.50 (m, 4H) 4.05 (t, 2H) 4.75 (s, 2H) 5.70 (s, 2H) 6.45 (s, 2H)

### Preparation 33

### 5-(Pyrrolidin-1-yl)-1,4-dihydro-2H-pyrano[4″,3″:4′,5′]pyrido[3′,2′:4,5]thieno[3,2-d]pyrimidin-8(9H)-one

1-Amino-5-(pyrrolidin-1-yl)-8,9-dihydro-6H-pyrano[4,3-d]thieno[2,3-b]pyridine-2-carboxamide (0.88 g, 2.78 mmol, see Preparation 61) is suspended is ethyl orthoformate (16 ml) and acid p-toluensulfonic monohydrate (0.03 g, 0.15 mmol) is added. This mixture is refluxed for 4 h. Once at room temperature, a solid precipitates. After leaving the mixture in an ice bath, 0.63 g of the final product is isolated by filtration and subsequent drying. Yield=69%.

¹H NMR (300 MHz, CHLOROFORM-D) δ ppm 1.95 (m, 4H) 2.45 (s, 2H) 3.50 (m, 4H) 3.95 (t, 2H) 4.80 (s, 2H) 8.15 (s, 1H) 12.65 (bs, 1H)

### Preparation 34

### 8-Chloro-5-(pyrrolidin-1-yl)-1,4-dihydro-2H-pyrano[4″,3″:4′,5′]pyrido[3′,2′:4,5]thieno[3,2-d]pyrimidine

5-(Pyrrolidin-1-yl)-1,4-dihydro-2H-pyrano[4″,3″:4′,5′]pyrido[3′,2′:4,5]thieno[3,2-d]pyrimidin-8(9H)-one (0.63 g, 1.92 mmol, see Preparation 62) is suspended in phosphorous oxychloride (8 ml) and this mixture is refluxed for 90 minutes. The solvent is evaporated under vacuum. To the residue ice is added and then NaOH 2N dropwise until the pH becomes basic. This aqueous phase is extracted repeatedly with chloroform. The organic phase is washed with brine, dried over magnesium sulfate, filtered and evaporated. 0.67 g of a brownish solid is obtained, whose ¹HNMR is consistent with the proposed final structure. Quantitative yield.

¹H NMR (300 MHz, CHLOROFORM-D) δ ppm 2.00 (m, 4H) 2.65 (m, 6H) 4.10 (t, 2H) 4.90 (s, 2H) 8.95 (s, 1H)

### Preparation 35

### 8-Chloro-5-propyl-1,4-dihydro-2H-pyrano[4″,3″:4′,5′]pyrido[3′,2′:4,5]thieno[3,2-d]pyrimidine

5-Propyl-1,4-dihydro-2H-pyrano[4″,3″:4′,5′]pyrido[3′,2′:4,5]thieno[3,2-d]pyrimidin-8(9H)-one (0.10 g, 0.30 mmol, purchased at Chemical Diversity, ref.nr.CDI-4576-0157) is suspended in phosphorous oxychloride (1 ml) and this mixture is refluxed for 90 minutes. The solvent is evaporated under vacuum. To the residue ice is added and then NaOH 2N dropwise until the pH becomes basic. This aqueous phase is extracted repeatedly with chloroform. The organic phase is washed with brine, dried over magnesium sulfate, filtered and evaporated. 0.088 g of a yellowish solid is obtained, whose ¹HNMR is consistent with the proposed final structure. Yield=83%.

¹H NMR (300 MHz, CHLOROFORM-D) δ ppm 1.05 (t, 3H) 1.40 (s, 6H) 1.85 (m, 2H) 2.80 (t, 2H) 3.60 (s, 2H) 4.95 (s, 2H) 9.10 (s, 1H)

### Preparation 36

### 5-Butyl-8-chloro-1,4-dihydro-2H-pyrano[4″,3″:4′,5′]pyrido[3′,2′:4,5]thieno[3,2-d]pyrimidine

5-Butyl-1,4-dihydro-2H-pyrano[4″,3″:4′,5′]pyrido[3′,2′:4,5]thieno[3,2-d]pyrimidin-8(9H)-one (0.10 g, 0.29 mmol, purchased at Chemical Diversity, ref.nr.CDI-4576-0163) is suspended in phosphorous oxychloride (1 ml) and this mixture is refluxed for 90 minutes. The solvent is evaporated under vacuum. To the residue ice is added and then NaOH 2N dropwise until the pH becomes basic. This aqueous phase is extracted repeatedly with chloroform. The organic phase is washed with brine, dried over magnesium sulfate, filtered and evaporated. 0.11 g of a yellowish solid is obtained, whose ¹HNMR is consistent with the proposed final structure. Yield=100%.

¹H NMR (300 MHz, CHLOROFORM-D) δ ppm 0.95 (t, 3H) 1.40 (s, 6H) 1.80 (m, 2H) 2.80 (t, 2H) 3.60 (s, 2H) 4.10 (m, 2H) 4.95 (s, 2H) 9.05 (s, 1H)

### Preparation 37

### 8-Chloro-5-isobutyl-1,4-dihydro-2H-pyrano[4″,3″:4′,5′]pyrido[3′,2′:4,5]thieno[3,2-d]pyrimidine

5-Isobutyl-1,4-dihydro-2H-pyrano[4″,3″:4′, 5′]pyrido[3′,2′:4,5]thieno[3,2-d]pyrimidin-8(9H)-one (0.10 g, 0.29 mmol, purchased at Chemical Diversity, ref.nr.CDI-4576-0167) is suspended in phosphorous oxychloride (2 ml) and this mixture is refluxed for 90 minutes. The solvent is evaporated under vacuum. To the residue ice is added and then NaOH 2N dropwise until the pH becomes basic. This aqueous phase is extracted repeatedly with chloroform. The organic phase is washed with brine, dried over magnesium sulfate, filtered and evaporated. 0.10 g of a solid is obtained, whose ¹HNMR is consistent with the proposed final structure. Yield=97%.

### Preparation 38

### 8-Dimethylamino-6-mercapto-3,3-dimethyl-3,4-dihydro-1H-pyrano[3,4-c]pyridine-5-carbonitrile

The product resulting from preparation 4 (1.50 g, 5.90 mmol) is suspended in ethanol (1.6 ml) and dimethylamine (6.0 ml, 5.6M solution in ethanol, 33.6 mmol) is added. The reaction mixture is heated at 85° C. under nitrogen at a pressure vessel for 16 h. The solvent is evaporated under vacuum and the resulting residue is purified by flash chromatography eluting with a mixture of CH₂Cl₂:MeOH 9:1. 0.45 g of the final compound is obtained. Yield=29%

¹H NMR (300 MHz, CHLOROFORM-D) δ ppm 1.30 (s, 6H) 1.60 (m, 1H) 2.70 (s, 2H) 3.05 (s, 6H) 4.60 (s, 2H)

### Preparation 39

### 1-Amino-5-dimethylamino-8,8-dimethyl-8,9-dihydro-6H-pyrano[4,3-a]thieno[2,3-b]pyridine-2-carboxamide

To a suspension of 8-dimethylamino-6-mercapto-3,3-dimethyl-3,4-dihydro-1H-pyrano[3,4-c]pyridine-5-carbonitrile (0.45 g, 1.71 mmol, see Preparation 38) in ethanol (25 ml), potassium carbonate (0.57 g, 4.10 mmol) and 2-chloroacetamide (0.18 g, 1.88 mmol) are added, and the reaction mixture is then refluxed under nitrogen for 5 h and then left at room temperature overnight. The solvent is evaporated under vacuum and water is added to the residue. The aqueous phase is extracted twice with chloroform. The organic phase is washed with water and brine, dried over MgSO₄, filtered and evaporated to dryness. 0.55 g of the desired final compound are obtained, pure enough to perform the next synthetic step. The ¹HNMR is consistent with the final product. Quantitative yield.

¹H NMR (300 MHz, CHLOROFORM-D) δ ppm 1.40 (s, 6H) 2.90 (s, 6H) 3.10 (s, 2H) 4.70 (s, 2H) 5.25 (s, 2H) 6.35 (s, 2H)

### Preparation 40

### 5-Dimethylamino-2,2-dimethyl-1,4-dihydro-2H-pyrano[4″,3″:4′,5′]pyrido[3′,2′:4,5]thieno[3,2-a]pyrimidin-8(9H)-one.

1-Amino-5-dimethylamino-8,8-dimethyl-8,9-dihydro-6H-pyrano[4,3-d]thieno[2,3-b]pyridine-2-carboxamide (0.55 g, 1.71 mmol, see Preparation 39) is suspended in ethyl orthoformate (15 ml) and p-toluensulfonic acid hydrate (0.03 g, 0.17 mmol) is added. This mixture is heated under reflux for 4 h. Then the reaction mixture is allowed to reach room temperature and left in an ice bath for two hours. The precipitated formed is filtered and washed with ethyl ether. After drying it weighs 0.44 g and its ¹H-NMR is consistent with the desired compound. Yield=78%

¹H NMR (300 MHz, CHLOROFORM-D) δ ppm 1.40 (s, 6H) 3.0 (s, 6H) 3.45 (s, 2H) 4.80 (s, 2H) 8.20 (s, 1H) 11.9 (s, 1H)

### Preparation 41

### 8-Chloro-5-dimethylamino-2,2-dimethyl-1,4-dihydro-2H-pyrano[4″,3″:4′,5′]pyrido[3′,2′:4,5]thieno[3,2-c]pyrimidine.

The final product of preparation 40 (0.44 g, 1.34 mmol) is suspended in phosphorous oxychloride (7 ml) and heated to reflux for 3 h. The excess of phosphorous oxychloride is evaporated under vacuum and the residue redissolved between chloroform and a cooled solution of 2N NaOH. The aqueous phase is extracted twice with chloroform and the organic phases are washed with NaOH 2N, water and brine, dried over magnesium sulfate, filtered and the solvent evaporated. 0.45 g of an oil is obtained, which ¹H-RMN is consistent with the desired compound. Yield=97%.

¹H NMR (300 MHz, CHLOROFORM-D) δ ppm 1.40 (s, 6H) 3.05 (s, 6H) 3.50 (s, 2H) 4.80 (s, 2H) 9.0 (s, 1H)

### Preparation 42

### 8-(Benzylmethylamino)-6-mercapto-3,3-dimethyl-3,4-dihydro-1H-pyrano[3,4-c]pyridin-5-carbonitrile

The product resulting from preparation 4 (5.38 g, 21.32 mmol) is suspended in ethanol (20 ml) and benzylmethylamine (16.5 ml, 127.92 mmol) is added. The reaction mixture is heated for 48 h at 90° C. under nitrogen in a pressure vessel. The solvent is evaporated and the residue is passed through a flash chromatography column, eluting first with dichloromethane and then with the mixture CH₂Cl₂:MeOH 98:2. 3.18 g of the final compound are obtained.

Yield=44%.

LRMS: m/z 340 (M+1)+

### Preparation 43

### 1-Amino-5-benzylmethylamino-8,8-dimethyl-8,9-dihydro-6H-pyrano[4,3-d]thieno[2,3-b]pyridin-2-carboxamide

To a suspension of 8-(benzylmethylamino)-6-mercapto-3,3-dimethyl-3,4-dihydro-1H-pyrano[3,4-c]pyridin-5-carbonitrile (3.18 g, 9.37 mmol, see Preparation 42) in ethanol (95 ml), potassium carbonate (2.59 g, 18.74 mmol) and 2-chloroacetamide (0.96 g, 10.31 mmol) are added, and the reaction mixture is then refluxed overnight under nitrogen. The solvent is evaporated under vacuum and water is added to the residue. A solid precipitates, which is filtered off and washed with Et₂O. It weighs 1.55 g and by ¹HRMN is the final compound. After extraction of the aqueous phase with ethyl ether, the organic phase is washed with water and brine, dried over magnesium sulfate, filtered and evaporated under vacuum. 1.37 g more of the final compound are isolated. Its ¹H-RMN is consistent with the proposed structure. Yield=78%.

¹H NMR (200 MHz, CDCl₃) δ ppm 1.4 0 (s, 6H) 2.80 (s, 3H) 3.20 (s, 2H) 4.40 (s, 2H) 4.80 (s, 2H) 5.40 (bs, 2H) 6.4 (bs, 2H) 7.40 (m, 5H)

### Preparation 44

### 2,2-Dimethyl-5-benzylmethylamino-1,4-dihydro-2H-pyrano[4″,3″:4′,5′]pyrido[3′,2′:4,5]thieno[3,2-d]pyrimidin-8(9H)-one

1-Amino-5-benzylmethylamino-8,8-dimethyl-8,9-dihydro-6H-pyrano[4,3-d]thieno[2,3-b]pyridin-2-carboxamide (2.93 g, 7.39 mmol, see Preparation 43) is suspended in ethyl orthoformate (65 ml) and p-toluensulfonic acid hydrate (0.15 g, 0.79 mmol) is added. This mixture is heated under reflux for 4 h. Once at room temperature, 1.24 g of the final compound precipitates, which is filtered. The liquid phase is evaporated under vacuum and the residue is purified by flash chromatography, eluting first with dichloromethane/methanol 98:2 and then with dichloromethane/methanol 9:1. 0.57 g more of the desired final compound are isolated. Global yield=60%.

¹H NMR (200 MHz, CDCl₃) δ ppm 1.4 (s, 6H) 2.90 (s, 3H) 3.50 (s, 2H) 4.50 (s, 2H) 4.85 (s, 2H) 7.40 (m, 5H) 8.15 (s, 1H) 12.5 (bs, 1H)

### Preparation 45

### N-Benzyl-8-chloro-N,2,2-trimethyl-1,4-dihydro-2H-pirano[4″,3″:4′,5′]pyrido[3′,2′:4,5]thieno[3,2-d]pyrimidine-5-amine

The final product of preparation 44 (1.81 g, 4.45 mmol) is suspended in phosphorous oxychloride (23.3 ml) and heated at 100° C. for 3 h. Once at room temperature, it is poured over NaOH 8N/ice. The mixture is extracted with ethyl acetate, washed with water and brine, dried over sodium sulfate, filtered and the solvent evaporated. By grinding the residue with Et₂O. 1.01 g of the final compound are obtained as a brownish solid. The ¹HNMR is consistent with the desired final product. Yield=53%.

¹H NMR (200 MHz, CDCl₃) δ ppm 1.4 (s, 6H) 3.0 (s, 3H) 3.55 (s, 2H) 4.60 (s, 2H) 4.85 (s, 2H) 7.40 (m, 5H) 9.0 (s, 1H)

### Preparation 46

### N⁵-Benzyl-N⁵,2,2-trimethyl-N⁸-(2-morpholin-4-ylethyl)-1,4-dihydro-2H-pyrano[4″,3″:4′,5′]pyrido[3′,2′:4,5]thieno[3,2-d]pyrimidin-5,8-diamine

N-Benzyl-8-chloro-N,2,2-trimethyl-1,4-dihydro-2H-pirano[4″,3″:4′,5′]pyrido[3′,2′:4,5]thieno[3,2-d]pyrimidine-5-amine (0.25 g, 0.59 mmol, see Preparation 45) is suspended in ethanol (15 ml) and (2-morpholin-4-ylethyl)amine (0.38 g, 2.95 mmol) is added. The mixture is refluxed 48 h and then allowed to cool to room temperature. The solvent is evaporated under reduced pressure and the residue is purified by flash chromatography eluting first with dichloromethane and then with dichloromethane/methanol 99:1 and finally 98:2. 200 mg of the final product have been isolated. Its ¹HNMR is consistent with the desired final compound. Yield=65%.

¹H NMR (300 MHz, CHLOROFORM-D) δ ppm 1.4 (s, 6H) 2.55 (m, 4H) 2.7 (t, J=6.6 Hz, 2H) 2.9 (s, 3H) 3.6 (s, 2H) 3.75 (m, 6H) 4.45 (s, 2H) 4.85 (s, 2H) 5.6 (t, 1H) 7.4 (m, 5H) 8.7 (s, 1H)

### Preparation 47

### N⁵-Benzyl-N⁵,2,2-trimethyl-N⁸-(pyridin-3-ylmethyl)-1,4-dihydro-2H-pyrano[4″,3″:4′,5′]pyrido[3′,2′:4,5]thieno[3,2-d]pyrimidin-5,8-diamina

Obtained (72%) from the title compound of Preparation 45 and (pyridine-3-ylmethyl)amine following the experimental procedure described in Preparation 46.

¹H NMR (300 MHz, CHLOROFORM-D) δ ppm 1.4 (s, 6H) 2.90 (s, 3H) 3.60 (s, 2H) 4.45 (s, 2H) 4.85 (s, 2H) 4.90 (d, 2H) 5.50 (t, 1H) 7.35 (m, 6H) 7.75 (d, 1H) 8.55 (m, 1H) 8.65 (m, 1H) 8.75 (s, 1H)

### Preparation 48

### 1-[3-({5-[Benzyl(methyl)amino]-2,2-dimethyl-1,4-dihydro-2H-pyrano[4″,3″:4′,5′]pyrido[3′,2′:4,5]thieno[3,2-d]pyrimidin-8-yl}amino)propyl]pyrrolidin-2-one

Obtained (55%) from the title compound of Preparation 45 and 1-(3-aminopropyl)pyrrolidin-2-one following the experimental procedure described in Preparation 46.

¹H NMR (300 MHz, CHLOROFORM-D) δ ppm 1.4 (s, 6H) 1.90 (m, 2H) 2.1 (m, 2H) 2.50 (t, 2H) 2.90 (s, 3H) 3.45 (m, 4H) 3.60 (s, 2H) 3.65 (m, 2H) 4.45 (s, 2H) 4.85 (s, 2H) 6.45 (t, 1H) 7.40 (m, 5H) 8.70 (s, 1H)

### Preparation 49

### N⁵-Benzyl-N⁵,2,2-trimethyl-N⁸-(2-morpholin-4-ylethyl)-N⁸-(pyridin-3-ylmethyl)-1,4-dihydro-2H-pyrano[4″,3″:4′,5′]pyrido[3′,2′:4,5]thieno[3,2-d]pyrimidin-5,8-diamine

Obtained (42%) from the title compound of Preparation 45 and (2-morpholin-4-ylethyl)-pyridin-3-ylmethylamine following the experimental procedure described in Preparation 46.

¹H NMR (300 MHz, CHLOROFORM-D) δ ppm 1.4 (s, 6H) 2.55 (m, 4H) 2.75 (t, 2H) 2.90 (s, 3H) 3.70 (m, 4H) 3.90 (t, 2H) 4.45 (s, 2H) 4.75 (s, 2H) 4.85 (s, 2H) 5.20 (s, 2H) 7.30 (m, 5H) 7.65 (d, 1H) 7.75 (d, 1H) 8.50 (m, 1H) 8.60 (bs, 1H) 8.75 (s, 1H).

### EXAMPLES

### Examples 1-54

The compounds of examples 1 to 54, showing activity as inhibitors of phosphodiesterase 4, have been obtained from libraries of compounds which are commercially available from the following companies:
Specs
Delftechpark 30
2628 XH Delft
The Netherlands
Web site: www.specs.net
InterBioScreen Ltd.,
121019 Moscow
P.O. Box 218
RUSSIA
Web site: www.ibscreen.com
Pharmeks Ltd.
105318
Mironovskaya str. 10A
Moscow, RUSSIA

The table below indicates for each compound the library from which it has been obtained, the reference number of the compound within the library and the IUPAC name of the compound:

| Exemple | Library | Reference | Compound |
|---|---|---|---|
| 1 | SPECS | AL-281/40711520 | 2,2-Dimethyl-5-morpholin-4-yl-N-(2- |
| | | | phenetylethyl)-1,4-dihydro-2H- |
| | | | pyrano[4″,3″:4′,5′]pyrido[3′,2′:4,5]thieno[3,2- |
| | | | d]pyrimidin-8-amine |
| 2 | SPECS | AL-281/40711521 | 2,2-Dimethyl-5-morpholin-4-yl-N-(4- |
| | | | methylpiperidin-1-yl)-1,4-dihydro-2H- |
| | | | pyrano[4″,3″:4′,5′]pyrido[3′,2′:4,5]thieno[3,2- |
| | | | d]pyrimidin-8-amine |
| 3 | SPECS | AL-281/40711524 | 2,2-Dimethyl-5-morpholin-4-yl-N-(2- |
| | | | diethylaminoethyl)-1,4-dihydro-2H- |
| | | | pyrano[4″,3″:4′,5′]pyrido[3′,2′:4,5]thieno[3,2- |
| | | | d]pyrimidin-8-amine |
| 4 | SPECS | AL-281/40711525 | 2,2-Dimethyl-5-morpholin-4-yl-N-butyl-N- |
| | | | methyl-1,4-dihydro-2H- |
| | | | pyrano[4″,3″:4′,5′]pyrido[3′,2′:4,5]thieno[3,2- |
| | | | d]pyrimidin-8-amine |
| 5 | SPECS | AL-281/47011529 | 2,2-Dimethyl-5-morpholin-4-yl-N-(2- |
| | | | tetrahydrofurylmethyl)-1,4-dihydro-2H- |
| | | | pyrano[4″,3″:4′,5′]pyrido[3′,2′:4,5]thieno[3,2- |
| | | | d]pyrimidin-8-amine |
| 6 | SPECS | AL-281/47011530 | 2,2-Dimethyl-5-morpholin-4-yl-N-butyl- |
| | | | 1,4-dihydro-2H- |
| | | | pyrano[4″,3″:4′,5′]pyrido[3′,2′:4,5]thieno[3,2- |
| | | | d]pyrimidin-8-amine |
| 7 | SPECS | AL-281/40711533 | 2,2-Dimethyl-5-morpholin-4-yl-N-(3- |
| | | | diethylaminopropyl)-1,4-dihydro-2H- |
| | | | pyrano[4″,3″:4′,5′]pyrido[3′,2′:4,5]thieno[3,2- |
| | | | d]pyrimidin-8-amine |
| 8 | INTERBIOSCREEN | STOCK1S-21298 | 2,2-Dimethyl-5,8-dimorpholin-4-yl-1,4- |
| | | | dihydro-2H- |
| | | | pyrano[4″,3″:4′,5′]pyrido[3′,2′:4,5]thieno[3,2- |
| | | | d]pyrimidine |
| 9 | INTERBIOSCREEN | STOCK1S-30189 | 2,2-Dimethyl-5-morpholin-4-yl-N- |
| | | | cyclohexyl-1,4-dihydro-2H- |
| | | | pyrano[4″,3″:4′,5′]pyrido[3′,2′:4,5]thieno[3,2- |
| | | | d]pyrimidin-8-amine |
| 10 | INTERBIOSCREEN | STOCK1S-37343 | 2,2-Dimethyl-5-morpholin-4-yl-N,N- |
| | | | diethyl-1,4-dihydro-2H- |
| | | | pyrano[4″,3″:4′,5′]pyrido[3′,2′:4,5]thieno[3,2- |
| | | | d]pyrimidin-8-amine |
| 11 | INTERBIOSCREEN | STOCK1S-37042 | 2,2-Dimethyl-5-morpholin-4-yl-8-(2- |
| | | | phenylhydrazino)-1,4-dihydro-2H- |
| | | | pyrano[4″,3″:4′,5′]pyrido[3′,2′:4,5]thieno[3,2- |
| | | | d]pyrimidine |
| 12 | INTERBIOSCREEN | STOCK1S-37052 | 2,2-Dimethyl-5-morpholin-4-yl-N-pentyl- |
| | | | 1,4-dihydro-2H- |
| | | | pyrano[4″,3″:4′,5′]pyrido[3′,2′:4,5]thieno[3,2- |
| | | | d]pyrimidin-8-amine |
| 13 | INTERBIOSCREEN | STOCK1S-37479 | 2,2-Dimethyl-5-morpholin-4-yl-1,4- |
| | | | dihydro-2H- |
| | | | pyrano[4″,3″:4′,5′]pyrido[3′,2′:4,5]thieno[3,2- |
| | | | d]pyrimidin-8-amine |
| 14 | INTERBIOSCREEN | STOCK1S-37493 | 2,2-Dimethyl-5-morpholin-4-yl-N-allyl- |
| | | | 1,4-dihydro-2H- |
| | | | pyrano[4″,3″:4′,5′]pyrido[3′,2′:4,5]thieno[3,2- |
| | | | d]pyrimidin-8-amine |
| 15 | INTERBIOSCREEN | STOCK1S-38197 | 2,2-Dimethyl-5-propyl-N-(3- |
| | | | hydroxypropyl)-1,4-dihydro-2H- |
| | | | pyrano[4″,3″:4′,5′]pyrido[3′,2′:4,5]thieno[3,2- |
| | | | d]pyrimidin-8-amine |
| 16 | INTERBIOSCREEN | STOCK1S-57008 | 2,2-Dimethyl-5-morpholin-4-yl-N-(3- |
| | | | hydroxypropyl)-1,4-dihydro-2H- |
| | | | pyrano[4″,3″:4′,5′]pyrido[3′,2′:4,5]thieno[3,2- |
| | | | d]pyrimidin-8-amine |
| 17 | INTERBIOSCREEN | STOCK1S-57293 | 2,2-Dimethyl-5-butyl-4-yl-N-(2-morpholin- |
| | | | 4-ylethyl)-1,4-dihydro-2H- |
| | | | pyrano[4″,3″:4′,5′]pyrido[3′,2′:4,5]thieno[3,2- |
| | | | d]pyrimidin-8-amine |
| 18 | INTERBIOSCREEN | STOCK1S-61240 | 2,2-Dimethyl-5-phenyl-4-yl-N-(2- |
| | | | dimethylaminoethyl)-1,4-dihydro-2H- |
| | | | pyrano[4″,3″:4′,5′]pyrido[3′,2′:4,5]thieno[3,2- |
| | | | d]pyrimidin-8-amine |
| 19 | INTERBIOSCREEN | STOCK1S-78393 | 2,2-Dimethyl-5-morpholin-4-yl-N-(pyridin- |
| | | | 2-yl)-1,4-dihydro-2H- |
| | | | pyrano[4″,3″:4′,5′]pyrido[3′,2′:4,5]thieno[3,2- |
| | | | d]pyrimidin-8-amine |
| 20 | INTERBIOSCREEN | STOCK1S-91007 | 2,2-Dimethyl-5-morpholin-4-yl-N-(2- |
| | | | morpholin-4-ylethyl)-1,4-dihydro-2H- |
| | | | pyrano[4″,3″:4′,5′]pyrido[3′,2′:4,5]thieno[3,2- |
| | | | d]pyrimidin-8-amine |
| 21 | INTERBIOSCREEN | STOCK2S-07331 | 2,2-Dimethyl-N-(1-methyl-3- |
| | | | phenylpropyl)-5-morpholin-4-yl-1,4- |
| | | | dihydro-2H- |
| | | | pyrano[4″,3″:4′,5′]pyrido[3′,2′:4,5]thieno[3,2- |
| | | | d]pyrimidin-8-amine |
| 22 | INTERBIOSCREEN | STOCK2S-09502 | 2,2-Dimethyl-5-isobutyl-4-yl-N-(2- |
| | | | morpholin-4-ylethyl)-1,4-dihydro-2H- |
| | | | pyrano[4″,3″:4′,5′]pyrido[3′,2′:4,5]thieno[3,2- |
| | | | d]pyrimidin-8-amine |
| 23 | INTERBIOSCREEN | STOCK2S-16966 | 2,2-Dimethyl-5-furan-2-yl-N-(2- |
| | | | morpholin-4-ylethyl)-1,4-dihydro-2H- |
| | | | pyrano[4″,3″:4′,5′]pyrido[3′,2′:4,5]thieno[3,2- |
| | | | d]pyrimidin-8-amine |
| 24 | INTERBIOSCREEN | STOCK2S-69776 | 2,2-Dimethyl-5-pyrrolidin-1-yl-N-benzyl- |
| | | | 1,4-dihydro-2H- |
| | | | pyrano[4″,3″:4′,5′]pyrido[3′,2′:4,5]thieno[3,2- |
| | | | d]pyrimidin-8-amine |
| 25 | INTERBIOSCREEN | STOCK2S-75256 | 2,2-Dimethyl-5-morpholin-4-yl-N-benzyl- |
| | | | N-methyl-1,4-dihydro-2H- |
| | | | pyrano[4″,3″:4′,5′]pyrido[3′,2′:4,5]thieno[3,2- |
| | | | d]pyrimidin-8-amine |
| 26 | INTERBIOSCREEN | STOCK2S-92629 | 2,2-Dimethyl-5-pyrrolidin-1-yl-8- |
| | | | morpholin-4-yl-1,4-dihydro-2H- |
| | | | pyrano[4″,3″:4′,5′]pyrido[3′,2′:4,5]thieno[3,2- |
| | | | d]pyrimidine |
| 27 | INTERBIOSCREEN | STOCK2S-94368 | 2,2-Dimethyl-5-pyrrolidin-1-yl-N-(2- |
| | | | morpholin-4-ylethyl)-1,4-dihydro-2H- |
| | | | pyrano[4″,3″:4′,5′]pyrido[3′,2′:4,5]thieno[3,2- |
| | | | d]pyrimidin-8-amine |
| 28 | INTERBIOSCREEN | STOCK2S-16294 | 2,2-Dimethyl-5-morpholin-4-yl-N-furan-2- |
| | | | ylmethyl-1,4-dihydro-2H- |
| | | | pyrano[4″,3″:4′,5′]pyrido[3′,2′:4,5]thieno[3,2- |
| | | | d]pyrimidin-8-amine |
| 29 | INTERBIOSCREEN | STOCK2S-94784 | 2,2-Dimethyl-5-pyrrolidin-1-yl-N- |
| | | | phenetyl-1,4-dihydro-2H- |
| | | | pyrano[4″,3″:4′,5′]pyrido[3′,2′:4,5]thieno[3,2- |
| | | | d]pyrimidin-8-amine |
| 30 | INTERBIOSCREEN | STOCK3S-07116 | 2,2-Dimethyl-5-pyrrolidin-1-yl-N-(3- |
| | | | dimethylaminopropyl)-1,4-dihydro-2H- |
| | | | pyrano[4″,3″:4′,5′]pyrido[3′,2′:4,5]thieno[3,2- |
| | | | d]pyrimidin-8-amine |
| 31 | INTERBIOSCREEN | STOCK3S-11445 | 2,2-Dimethyl-5-pyrrolidin-1-yl-N- |
| | | | isopentyl-1,4-dihydro-2H- |
| | | | pyrano[4″,3″:4′,5′]pyrido[3′,2′:4,5]thieno[3,2- |
| | | | d]pyrimidin-8-amine |
| 32 | INTERBIOSCREEN | STOCK3S-12659 | 2,2-Dimethyl-N-(1-methyl-3- |
| | | | phenylpropyl)-5-pyrrolidin-1-yl-1,4- |
| | | | dihydro-2H- |
| | | | pyrano[4″,3″:4′,5′]pyrido[3′,2′:4,5]thieno[3,2- |
| | | | d]pyrimidin-8-amine |
| 33 | INTERBIOSCREEN | STOCK3S-21027 | 2,2-Dimethyl-5-morpholin-4-yl-N-(2- |
| | | | hydroxyethyl)-N-benzyl-1,4-dihydro-2H- |
| | | | pyrano[4″,3″:4′,5′]pyrido[3′,2′:4,5]thieno[3,2- |
| | | | d]pyrimidin-8-amine |
| 34 | INTERBIOSCREEN | STOCK3S-21213 | 2,2-Dimethyl-5-pyrrolidin-1-yl-N- |
| | | | tetrahydrofuran-2-yl-1,4-dihydro-2H- |
| | | | pyrano[4″,3″:4′,5′]pyrido[3′,2′:4,5]thieno[3,2- |
| | | | d]pyrimidin-8-amine |
| 35 | INTERBIOSCREEN | STOCK3S-27128 | 2,2-Dimethyl-5-pyrrolidin-1-yl-N-pentyl- |
| | | | 1,4-dihydro-2H- |
| | | | pyrano[4″,3″:4′,5′]pyrido[3′,2′:4,5]thieno[3,2- |
| | | | d]pyrimidin-8-amine |
| 36 | INTERBIOSCREEN | STOCK4S-70521 | 2,2-dimethyl-5-morpholin-4-yl-N-(pyridin- |
| | | | 3-ylmethyl)-1,4-dihydro-2H- |
| | | | pyrano[4″,3″:4′,5′]pyrido[3′,2′:4,5]thieno[3,2- |
| | | | d]pyrimidin-8-amine |
| 37 | INTERBIOSCREEN | STOCK4S-70441 | 2,2-dimethyl-5-morpholin-4-yl-N-(pyridin- |
| | | | 2-ylmethyl)-1,4-dihydro-2H- |
| | | | pyrano[4″,3″:4′,5′]pyrido[3′,2′:4,5]thieno[3,2- |
| | | | d]pyrimidin-8-amine |
| 38 | PHARMEKS | PHAR061682 | 2,2-dimethyl-N-(2-morpholin-4-ylethyl)-5- |
| | | | propyl-1,4-dihydro-2H- |
| | | | pyrano[4″,3″:4′,5′]pyrido[3′,2′:4,5]thieno[3,2- |
| | | | d]pyrimidin-8-amine |
| 39 | INTERBIOSCREEN | STOCK4S-19224 | 2-ethyl-2-methyl-5-morpholin-4-yl-N-(2- |
| | | | morpholin-4-ylethyl)-1,4-dihydro-2H- |
| | | | pyrano[4″,3″:4′,5′]pyrido[3′,2′:4,5]thieno[3,2- |
| | | | d]pyrimidin-8-amine |
| 40 | INTERBIOSCREEN | STOCK4S-74178 | 2,2-dimethyl-N-(pyridin-3-ylmethyl)-5- |
| | | | pyrrolidin-1-yl-1,4-dihydro-2H- |
| | | | pyrano[4″,3″:4′,5′]pyrido[3′,2′:4,5]thieno[3,2- |
| | | | d]pyrimidin-8-amine |
| 41 | INTERBIOSCREEN | STOCK4S-52807 | 2,2-dimethyl-N-(pyridin-2-ylmethyl)-5- |
| | | | pyrrolidin-1-yl-1,4-dihydro-2H- |
| | | | pyrano[4″,3″:4′,5′]pyrido′[3′,2′:4,5]thieno |
| | | | [3,2-d]pyrimidin-8-amine |
| 42 | INTERBIOSCREEN | STOCK4S-38280 | 5-(2-Furyl)-2,2-dimethyl-N-(pyridin-3- |
| | | | ylmethyl)-1,4-dihydro-2H- |
| | | | pyrano[4″,3″:4′,5′]pyrido[3′,2′:4,5]thieno[3,2- |
| | | | d]pyrimidin-8-amine |
| 43 | INTERBIOSCREEN | STOCK4S-54754 | 5-(2-Furyl)-N-(2-furylmethyl)-2,2- |
| | | | dimethyl-1,4-dihydro-2H- |
| | | | pyrano[4″,3″:4′,5′]pyrido[3′,2′:4,5]thieno[3,2- |
| | | | d]pyrimidin-8-amine |
| 44 | INTERBIOSCREEN | STOCK4S-53895 | 2,2-Dimethyl-5-methyl-N-(pyridin-3- |
| | | | ylmethyl)-1,4-dihydro-2H- |
| | | | pyrano[4″,3″:4′,5′]pyrido[3′,2′:4,5]thieno[3,2- |
| | | | d]pyrimidin-8-amine |
| 45 | INTERBIOSCREEN | STOCK4S-63321 | 2,2-Diemthyl-5-isobutyl-N-(2- |
| | | | furylmethyl)-1,4-dihydro-2H- |
| | | | pyrano[4″,3″:4′,5′]pyrido[3′,2′:4,5]thieno[3,2- |
| | | | d]pyrimidin-8-amine |
| 46 | INTERBIOSCREEN | STOCK4S-70642 | 2,2-Dimethyl-5-isopropyl-N-(pyridin-2- |
| | | | ylmethyl)-1,4-dihydro-2H- |
| | | | pyrano[4″,3″:4′,5′]pyrido[3′,2′:4,5]thieno[3,2- |
| | | | d]pyrimidin-8-amine |
| 47 | INTERBIOSCREEN | STOCK4S-78278 | 2,2-Dimethyl-5-isopropyl-N-(2- |
| | | | furylmethyl)-1,4-dihydro-2H- |
| | | | pyrano[4″,3″:4′,5′]pyrido[3′,2′:4,5]thieno[3,2- |
| | | | d]pyrimidin-8-amine |
| 48 | INTERBIOSCREEN | STOCK4S-81176 | 2,2-Dimethyl-5-isopropyl-N-(pyridin-3- |
| | | | ylmethyl)-1,4-dihydro-2H- |
| | | | pyrano[4″,3″:4′,5′]pyrido[3′,2′:4,5]thieno[3,2- |
| | | | d]pyrimidin-8-amine |
| 49 | INTERBIOSCREEN | STOCK4S-81410 | 2,2-Dimethyl-5-methyl-N-(pyridin-2- |
| | | | ylmethyl)-1,4-dihydro-2H- |
| | | | pyrano[4″,3″:4′,5′]pyrido[3′,2′:4,5]thieno[3,2- |
| | | | d]pyrimidin-8-amine |
| 50 | INTERBIOSCREEN | STOCK4S-82415 | 2,2-Dimethyl-5-morpholin-4-yl-N-(3- |
| | | | morpholin-4-ylpropyl)-1,4-dihydro-2H- |
| | | | pyrano[4″,3″:4′,5′]pyrido[3′,2′:4,5]thieno[3,2- |
| | | | d]pyrimidin-8-amine |
| 51 | INTERBIOSCREEN | STOCK4S-46232 | N-[2-(3,4-Dimethoxyphenyl)ethyl]-2,2- |
| | | | dimethyl-5-morpholin-4-yl-1,4-dihydro- |
| | | | 2H- |
| | | | pyrano[4″,3″:4′,5′]pyrido[3′,2′:4,5]thieno[3,2- |
| | | | d]pyrimidin-8-amine |
| 52 | INTERBIOSCREEN | STOCK4S-51127 | 5-(2-Furyl)-2,2-dimethyl-N-(pyridin-2- |
| | | | ylmethyl)-1,4-dihydro-2H- |
| | | | pyrano[4″,3″:4′,5′]pyrido[3′,2′:4,5]thieno[3,2- |
| | | | d]pyrimidin-8-amine |
| 53 | INTERBIOSCREEN | STOCK4S-39673 | N-[2-(3,4-Dimethoxyphenyl)ethyl]-2,2- |
| | | | dimethyl-5-isopropyl-1,4-dihydro-2H- |
| | | | pyrano[4″,3″:4′,5′]pyrido[3′,2′:4,5]thieno[3,2- |
| | | | d]pyrimidin-8-amine |
| 54 | INTERBIOSCREEN | STOCK4S-49472 | 1-[(5-Isopropyl-2,2-dimethyl-1,4-dihydro- |
| | | | 2H- |
| | | | pyrano[4″,3″:4′,5′]pyrido[3′,2′:4,5]thieno[3,2- |
| | | | d]pyrimidin-8-yl)amino]propan-2-ol |

### Example 55

### 2,2-Dimethyl-5-morpholin-4-yl-N-(pyridin-4-ylmethyl)-1,4-dihydro-2H-pyrano[4″,3″:4′,5′]pyrido[3′,2′:4,5]thieno[3,2-d]pyrimidin-8-amine

8-Chloro-2,2-dimethyl-5-morpholin-4-yl-1,4-dihydro-2H-pyrano[4″,3″:4′,5′]pyrido[3′,2′:4,5]thieno[3,2-d]pyrimidine (0.10 g, 0.26 mmol, see Preparation 8) is suspended in ethanol and pyridin-4-ylmethylamine (0.13 ml, 1.28 mmol) is added. The mixture is refluxed overnight and then allowed to cool at room temperature. At +5° C. a precipitate is formed, which is filtrated and washed with ethanol an ethyl ether. Once dried, it weights 0.015 g and its ¹H NMR is consistent with the initial chlorimine (15% recovered). The solvent is evaporated and the residue is purified by flash chromatography eluting with CH₂Cl₂/MeOH 98:2. 0.04 g of the desired compound are obtained. Yield=34%.

m.p. 238.0-239.7° C.

¹H NMR (300 MHz, DMSO-D6) δ ppm 1.32 (s, 6H) 3.20 (m, 4H) 3.50 (s, 2H) 3.77 (m, 4H) 4.71 (s, 2H) 4.77 (d, J=5.80 Hz, 2H) 7.33 (d, J=6.10 Hz, 2H) 8.44 (t, J=6.10 Hz, 1H) 8.49 (m, 2H) 8.56 (s, 1H)

### Example 56

### 2,2-Dimethyl-5-morpholin-4-yl-N-(2-piperidin-1-ylethyl)-1,4-dihydro-2H-pyrano[4″,3″:4′,5′]pyrido[3′,2′:4,5]thieno[3,2-d]pyrimidin-8-amine

Obtained (81%) from the title compound of Preparation 8 and 2-piperidin-1-ylethylamine following the experimental procedure described in Example 55.

m.p. 163.8-164.4° C.

¹H NMR (300 MHz, DMSO-D6) δ ppm 1.26 (m, 8H) 1.39 (m, 2H) 1.47 (m, 4H) 2.41 (m, 4H) 3.19 (m, 4H) 3.50 (s, 2H) 3.63 (m, 2H) 3.76 (m, 4H) 4.70 (s, 2H) 7.68 (t, J=5.95 Hz, 1H) 8.58 (s, 1H)

### Example 57

### N-(3-Methoxypropyl)-2,2-dimethyl-5-morpholin-4-yl-1,4-dihydro-2H-pyrano[4″,3″:4′,5′]pyrido[3′,2′:4,5]thieno[3,2-d]pyrimidin-8-amine

Obtained (44%) from the title compound of Preparation 8 and 3-methoxypropylamine following the experimental procedure described in Example 55.

m.p. 178.1-178.7° C.

¹H NMR (300 MHz, DMSO-D6) δ ppm 1.33 (m, 6H) 1.86 (m, 2H) 3.18 (m, 4H) 3.25 (s, 3H) 3.41 (t, J=6.10 Hz, 2H) 3.50 (s, 2H) 3.56 (m, 2H) 3.76 (m, 4H) 4.70 (s, 2H) 7.78 (t, J=5.19 Hz, 1H) 8.58 (s, 1H)

### Example 58

### N-(2-Methoxyethyl)-N,2,2-trimethyl-N-(2-morpholin-4-ylethyl)-1,4-dihydro-2H-pyrano[4″,3″:4′,5′]pyrido[3′,2′:4,5]thieno[3,2-d]pyrimidine-5,8-diamine.

8-Chloro-N-(2-methoxyethyl)-N,2,2-trimethyl-1,4-dihydro-2H-pyrano[4″,3″:4′,5′]pyrido[3′,2′:4,5]thieno[3,2-d]pyrimidine-5-amine (0.01 g, 0.24 mmol, see Preparation 12) is suspended in ethanol (5 ml) and (2-morpholino-4-ylethyl)amine (0.16 ml, 1.21 mmol) is added. The mixture is refluxed overnight and then allowed to cool to room temperature. The solvent is evaporated under vacuum and the residue is purified by chromatography, eluting first with dichloromethane and then with CH₂Cl₂:MeOH 98:2. 40 mg of the desired final product are obtained. Yield=34%.

m.p. 70.6-72.1° C.

¹H NMR (300 MHz, CHLOROFORM-D) δ ppm 1.42 (s, 6H) 1.65 (s, 2H) 2.55 (m, 4H) 2.71 (t, J=6.04 Hz, 2H) 3.03 (s, 3H) 3.36 (s, 3H) 3.49 (t, J=6.18 Hz, 2H) 3.62 (m, 2H) 3.74 (m, 6H) 4.81 (s, 2H) 5.56 (m, 1H) 8.70 (s, 1H)

### Example 59

### N-(2-Methoxyethyl)-N,2,2-trimethyl-N-(pyridin-3-ylmethyl)-1,4-dihydro-2H-pyrano[4″,3″:4′,5′]pyrido[3′,2′:4,5]thieno[3,2-d]pyrimidine-5,8-diamine.

Obtained (31%) from the title compound of Preparation 12 and pyridin-3-ylmethylamine following the experimental procedure described in Example 58.

m.p. 164.3-166.0° C.

¹H NMR (300 MHz, CHLOROFORM-D) δ ppm 1.43 (s, 3H) 1.61 (s, 3H) 3.03 (s, 3H) 3.35 (s, 3H) 3.50 (t, J=6.04 Hz, 2H) 3.62 (m, 4H) 4.81 (s, 2H) 4.93 (d, J=6.04 Hz, 2H) 5.04 (d, J=5.49 Hz, 1H) 7.30 (m, 1H) 7.75 (m, 1H) 8.56 (dd, J=4.81, 1.51 Hz, 1H) 8.69 (d, J=1.65 Hz, 1H) 8.73 (s, 1H)

### Example 60

### 2,2-Dimethyl-5-(4-methylpiperazin-1-yl)-N-(2-morpholin-4-ylethyl)-1,4-dihydro-2H-pyrano[4″,3″:4′,5′]pyrido[3′,2′:4,5]thieno[3,2-d]pyrimidin-8-amine.

8-Chloro-2,2-dimethyl-5-(4-methylpiperazin-1-yl)-1,4-dihydro-2H-pyrano[4″, 3″:4′,5′]pyrido[3′, 2′:4,5]thieno[3,2-d]pyrimidine (0.08 g, 0.20 mmol, see Preparation 16) is suspended in ethanol (5 ml) and (2-morpholino-4-ylethyl)amine (0.13 ml, 0.99 mmol) is added. The mixture is refluxed for 48 h and then allowed to cool to room temperature. The solvent is evaporated under vacuum and the residue is purified by chromatography, eluting first with CH₂Cl₂:MeOH 9:1. 40 mg of the desired final product are obtained. Yield=40%.

m.p. 171-171.8° C.

¹H NMR (300 MHz, CHLOROFORM-D) δ ppm 1.42 (s, 6H) 2.40 (s, 3H) 2.59 (m, 9H) 2.72 (t, J=5.95 Hz, 2H) 3.32 (m, 4H) 3.60 (s, 2H) 3.74 (m, 5H) 4.78 (s, 2H) 5.59 (m, J=4.88 Hz, 1H) 8.71 (s, 1H)

### Example 61

### 2,2-Dimethyl-5-(4-methylpiperazin-1-yl)-N-(3-morpholin-4-ylpropyl)-1,4-dihydro-2H-pyrano[4″,3″:4′,5′]pyrido[3′,2′:4,5]thieno[3,2-d]pyrimidin-8-amine

Obtained (92%) from the title compound of Preparation 16 and (3-morpholino-4-ylpropyl)amine following the experimental procedure described in Example 60.

m.p. 90.6-92.4° C.

¹H NMR (300 MHz, CHLOROFORM-D) δ ppm 1.40 (d, J=13.74 Hz, 6H) 1.89 (d, J=4.67 Hz, 3H) 2.40 (s, 3H) 2.64 (m, 7H) 3.32 (m, 4H) 3.60 (s, 2H) 3.76 (d, J=5.22 Hz, 2H) 3.96 (t, J=4.67 Hz, 4H) 4.78 (s, 2H) 8.69 (s, 1H)

### Example 62

### N-(2-Furylmethyl)-2,2-dimethyl-5-(4-methylpiperazin-1-yl)-1,4-dihydro-2H-pyrano[4″,3″:4′,5′]pyrido[3′,2′:4,5]thieno[3,2-d]pyrimidin-8-amine

Obtained (57%) from the title compound of Preparation 16 and (2-furylmethyl)amine following the experimental procedure described in Example 60.

m.p. 166.3-167.5° C.

¹H NMR (300 MHz, CHLOROFORM-D) δ ppm 1.43 (m, 6H) 2.39 (s, 3H) 2.62 (d, J=4.40 Hz, 4H) 3.31 (m, 4H) 3.60 (s, 2H) 4.78 (s, 2H) 4.89 (d, J=5.49 Hz, 2H) 5.02 (t, J=5.49 Hz, 1H) 6.36 (m, 2H) 7.41 (s, 1H) 8.76 (s, 1H)

### Example 63

### 2,2-Dimethyl-5-(4-methylpiperazin-1-yl)-N-(pyridin-4-ylmethyl)-1,4-dihydro-2H-pyrano[4″,3″:4′,5′]pyrido[3′,2′:4,5]thieno[3,2-d]pyrimidin-8-amine

Obtained (80%) from the title compound of Preparation 16 and (pyridin-4-ylmethyl)amine following the experimental procedure described in Example 60.

m.p. 197.1-198.3° C.

¹H NMR (300 MHz, CHLOROFORM-D) δ ppm 1.42 (s, 6H) 2.40 (s, 3H) 2.63 (s, 4H) δ 3.33 (m, 4H) 3.61 (s, 2H) 4.78 (s, 2H) 4.94 (d, J=6.10 Hz, 2H) 5.31 (d, J=6.10 Hz, 1H) 7.29 (m, 2H) 8.57 (d, J=4.58 Hz, 2H) 8.71 (s, 1H)

### Example 64

### 2,2-Dimethyl-5-(4-methylpiperazin-1-yl)-N-(pyridin-3-ylmethyl)-1,4-dihydro-2H-pyrano[4″,3″:4′,5′]pyrido[3′,2′:4,5]thieno[3,2-d]pyrimidin-8-amine

Obtained (47%) from the title compound of Preparation 16 and (pyridin-3-ylmethyl)amine following the experimental procedure described in Example 60.

m.p. 250.9-251.7° C.

¹H NMR (300 MHz, CHLOROFORM-D) δ ppm 1.42 (s, 6H) 2.39 (s, 3H) 2.62 (d, J=4.27 Hz, 4H) 3.32 (m, 4H) 3.60 (s, 2H) 4.78 (s, 2H) 4.93 (d, J=5.80 Hz, 2H) 5.13 (d, J=5.80 Hz, 1H) 7.29 (m, 1H) 7.76 (d, J=8.24 Hz, 1H) 8.56 (d, J=3.97 Hz, 1H) 8.69 (d, J=1.53 Hz, 1H) 8.74 (s, 1H)

### Example 65

### 2,2-Dimethyl-5-(4-methylpiperazin-1-yl)-N-(pyridin-2-ylmethyl)-1,4-dihydro-2H-pyrano[4″,3″:4′,5′]pyrido[3′,2′:4,5]thieno[3,2-d]pyrimidin-8-amine

Obtained (74%) from the title compound of Preparation 16 and (pyridin-2-ylmethyl)amine following the experimental procedure described in Example 60.

m.p. 218.1-219.4° C.

¹H NMR (300 MHz, CHLOROFORM-D) δ ppm 1.42 (s, 6H) 2.41 (s, 3H) 2.64 (s, 4H) 3.34 (d, J=3.97 Hz, 4H) 3.61 (s, 2H) 4.79 (s, 2H) 4.97 (d, J=4.27 Hz, 2H) 6.34 (s, 1H) 7.26 (m, 1H) 7.37 (d, J=7.93 Hz, 1H) 7.71 (t, J=7.63 Hz, 1H) 8.63 (d, J=4.88 Hz, 1H) 8.75 (s, 1H)

### Example 66

### 2,2-Dimethyl-5-(4-methylpiperazin-1-yl)-N-(2-pyridin-2-ylethyl)-1,4-dihydro-2H-pyrano[4″,3″:4′,5′]pyrido[3′,2′:4,5]thieno[3,2-d]pyrimidin-8-amine

Obtained (60%) from the title compound of Preparation 16 and (2-pyridin-2-ylethyl)amine following the experimental procedure described in Example 60.

m.p. 226.7-229.0° C.

¹H NMR (300 MHz, CHLOROFORM-D) δ ppm 1.37 (m, 6H) 2.41 (s, 3H) 2.64 (m, 4H) 3.19 (m, 2H) 3.33 (m, 4H) 3.60 (s, 2H) 4.06 (m, 2H) 4.78 (s, 2H) 6.41 (t, J=5.22 Hz, 1H) 7.20 (m, 2H) 7.64 (m, 1H) 8.63 (m, 1H) 8.71 (s, 1H)

### Example 67

### N-[3-(1H-Imidazol-1-yl)propyl]-2,2-dimethyl-5-(4-methylpiperazin-1-yl)-1,4-dihydro-2H-pyrano[4″,3″:4′,5′]pyrido[3′,2′:4,5]thieno[3,2-d]pyrimidin-8-amine

Obtained (35%) from the title compound of Preparation 16 and [(1H-imidazol-1-yl)propyl]amine following the experimental procedure described in Example 60.

m.p. 226.6-227.4° C.

¹H NMR (300 MHz, CHLOROFORM-D) δ ppm 1.42 (s, 6H) 2.26 (m, 2H) 2.40 (s, 3H) 2.62 (m, 4H) 3.32 (m, 4H) 3.60 (s, 2H) 3.72 (q, J=6.59 Hz, 2H) 4.12 (t, J=6.87 Hz, 2H) 4.78 (s, 2H) 4.86 (s, 1H) 6.99 (s, 1H) 7.11 (s, 1H) 7.56 (s, 1H) 8.71 (s, 1H)

### Example 68

### 2,2-Dimethyl-5-(4-methylpiperazin-1-yl)-N-[1-(tetrahydrofuran-3-ylmethyl)piperidin-4-yl]-1,4-dihydro-2H-pyrano[4″,3″:4′,5′]pyrido[3′,2′:4,5]thieno[3,2-d]pyrimidin-8-amine

Obtained (47%) from the title compound of Preparation 16 and [1-(tetrahydrofuran-3-ylmethyl)piperidin-4-yl]amine following the experimental procedure described in Example 60.

m.p. 170-170.9° C.

¹H NMR (300 MHz, CHLOROFORM-D) δ ppm 1.42 (m, 6H) 2.07 (m, 8H) 2.48 (d, J=26.37 Hz, 8H) 2.80 (s, 4H) 3.10 (s, 2H) 3.46 (m, 4H) 3.56 (m, 3H) 3.76 (m, 1H) 3.88 (m, 2H) 4.77 (s, 2H) 8.68 (s, 1H)

### Example 69

### 2,2-Dimethyl-N-(2-morpholin-4-ylethyl)-5-piperidin-1-yl-1,4-dihydro-2H-pyrano[4″,3″:4′,5′]pyrido[3′,2′:4,5]thieno[3,2-d]pyrimidin-8-amine

8-Chloro-2,2-dimethyl-5-(piperidin-1-yl)-1,4-dihydro-2H-pyrano[4″,3″:4′,5′]pyrido[3′,2′:4,5]thieno[3,2-d]pyrimidine (0.07 g, 0.18 mmol, see Preparation 20) is suspended in ethanol (5 ml) and (2-morpholino-4-ylethyl)amine (0.12 ml, 0.90 mmol) is added. The mixture is refluxed for 24 h and then allowed to cool to room temperature. The solvent is evaporated under vacuum and the residue is purified by chromatography, eluting first with CH₂Cl₂:MeOH 99:1 and then with CH₂Cl₂:MeOH 98:2. 69 mg of the desired final product are obtained. Yield=79%.

m.p. 157.9-158.5° C.

¹H NMR (300 MHz, CHLOROFORM-D) δ ppm 1.41 (d, J=6.04 Hz, 6H) 1.65 (m, 7H) 2.55 (m, 4H) 2.72 (t, J=6.04 Hz, 2H) 3.19 (m, 4H) 3.59 (s, 1H) 3.74 (m, 6H) 4.79 (s, 2H) 5.58 (s, 1H) 8.70 (s, 1H)

### Example 70

### 2,2-Dimethyl-5-piperidin-1-yl-N-(pyridin-3-ylmethyl)-1,4-dihydro-2H-pyrano[4″,3″:4′,5′]pyrido[3′,2′:4,5]thieno[3,2-d]pyrimidin-8-amine

Obtained (56%) from the title compound of Preparation 20 and (pyridine-3-ylmethyl)amine following the experimental procedure described in Example 69.

LRMS: m/z 461 (M+1)⁺

### Example 71

### 2,2-Dimethyl-N-(pyridin-4-ylmethyl)-5-pyrrolidin-1-yl-1,4-dihydro-2H-pyrano[4″,3″:4′,5′]pyrido[3′,2′:4,5]thieno[3,2-d]pyrimidin-8-amine

8-Chloro-2,2-dimethyl-5-(pyrrolidin-1-yl)-1,4-dihydro-2H-pyrano[4″,3″:4′,5′]pyrido[3′,2′:4,5]thieno[3,2-d]pyrimidine (0.08 g, 0.21 mmol, see Preparation 24) is suspended in ethanol (5 ml) and (pyridine-4-ylmethyl)amine (0.11 ml, 1.07 mmol) is added. The mixture is refluxed for 24 h and then allowed to cool at room temperature. The solvent is evaporated under vacuum and the residue is purified by chromatography, eluting first with CH₂Cl₂ and then with CH₂Cl₂:MeOH 99:1. 0.05 g of the desired product are obtained. Yield=52%.

m.p. 228.3-229.4° C.

¹H NMR (300 MHz, CHLOROFORM-D) δ ppm 1.42 (s, 6H) 1.98 (m, 4H) 3.50 (m, 2H) 3.64 (m, 4H) 4.92 (m, 4H) 5.10 (d, J=6.10 Hz, 1H) 7.29 (m, 2H) 8.57 (m, 2H) 8.67 (s, 1H)

### Example 72

### 2,2-Dimethyl-5-propyl-N-(pyridin-3-ylmethyl)-1,4-dihydro-2H-pyrano[4″,3″:4′,5′]pyrido[3′,2′:4,5]thieno[3,2-d]pyrimidin-8-amine

8-Chloro-5-propyl-1,4-dihydro-2H-pyrano[4″,3″:4′,5′]pyrido[3′,2′:4,5]thieno[3,2-d]pyrimidine (0.04 g, 0.13 mmol, see Preparation 35) is suspended in ethanol (5 ml) and pyridin-3ylmethylamine (0.07 ml, 0.63 mmol) is added. The mixture is heated at 85° C. for 24 h and then cooled to room temperature. The solvent is evaporated under vacuum and the residue is purified by flash chromatography, eluting with CH₂Cl₂:MeOH 99:1. 33 mg of the desired final product are obtained. Yield=62%.

m.p. 258.9-259.7° C.

¹H NMR (300 MHz, CHLOROFORM-D) d ppm 1.1 (t, 3H) 1.4 (s, 6H) 1.8 (m, 2H) 2.7 (m, 2H) 3.7 (s, 2H) 4.9 (d, J=3.0 Hz, 4H) 5.2 (t, J=5.6 Hz, 1H) 7.3 (m, 1H) 7.8 (m, 1H) 8.6 (dd, J=4.9, 1.6 Hz, 1H) 8.7 (d, J=2.5 Hz, 1H) 8.8 (m, 1H)

### Example 73

### 5-Butyl-N-(2-furylmethyl)-2,2-dimethyl-1,4-dihydro-2H-pyrano[4″,3″:4′,5′]pyrido[3′,2′:4,5]thieno[3,2-d]pyrimidin-8-amine

5-Butyl-8-chloro-1,4-dihydro-2H-pyrano[4″,3″:4′,5′]pyrido[3′,2′:4,5]thieno[3,2-d]pyrimidine (0.053 g, 0.15 mmol, see Preparation 36) is suspended in ethanol (6 ml) and furyl-2-ylmethylamine (0.065 ml, 0.73 mmol) is added. The mixture is heated at 85° C. for 24 h and the cooled to room temperature. The solvent is evaporated under vacuum and the residue is purified by flash chromatography, eluting with CH₂Cl₂:MeOH 99:1. 49 mg of the desired product are obtained. Yield=79%.

m.p. 66.1-68.5° C.

¹H NMR (300 MHz, CHLOROFORM-D) d ppm 1.0 (t, J=7.3 Hz, 3H) 1.4 (m, 6H) 1.6 (s, 2H) 1.7 (dd, J=15.5, 7.8 Hz, 2H) 2.8 (m, 2H) 3.6 (d, J=9.9 Hz, 2H) 4.9 (m, 4H) 5.1 (t, J=4.0 Hz, 1H) 6.4 (s, 2H) 7.4 (s, 1H) 8.8 (s, 1H)

### Example 74

### 5-Isobutyl-2,2-dimethyl-N-(pyridin-3-ylmethyl)-1,4-dihydro-2H-pyrano[4″,3″:4′,5′]pyrido[3′,2′:4,5]thieno[3,2-d]pyrimidin-8-amine

5-Isobutyl-8-chloro-1,4-dihydro-2H-pyrano[4″,3″:4′,5′]pyrido[3′,2′:4,5]thieno[3,2-d]pyrimidine (0.05 g, 0.14 mmol, see Preparation 37) is suspended in ethanol (5 ml) and pyridin-3-ylmethylamine (0.072 ml, 0.70 mmol) is added. The mixture is heated at 85° C. for 24 h and then cooled to room temperature. The solvent is evaporated under vacuum and the residue is purified by flash chromatography, eluting with CH₂Cl₂:MeOH 99:1. 35 mg of the desired final product are obtained. Yield=57%.

m.p. 244.3-245.2° C.

¹H NMR (300 MHz, CHLOROFORM-D) d ppm 1.0 (d, J=6.6 Hz, 6H) 1.4 (d, J=17.9 Hz, 6H) 2.3 (m, 1H) 2.6 (d, J=7.1 Hz, 2H) 3.7 (s, 2H) 4.9 (d, J=3.3 Hz, 4H) 5.2 (t, J=5.8 Hz, I H) 7.3 (m, 1H) 7.8 (dd, J=7.7, 1.6 Hz, 1H) 8.6 (m, 1H) 8.7 (s, 1H) 8.8 (s, 1H)

### Example 75

### 5-Morpholin-4-yl-N-(2-morpholin-4-ylethyl)-1,4-dihydro-2H-pyrano[4″,3″:4′,5′]pyrido[3′,2′:4,5]thieno[3,2-d]pyrimidin-8-amine

8-Chloro-5-(morpholin-1-yl)-1,4-dihydro-2H-pyrano[4″,3″:4′,5′]pyrido[3′,2′:4,5]thieno[3,2-d]pyrimidine (0.05 g, 0.14 mmol, see Preparation 30) is suspended in ethanol (5 ml) and (2-morpholin-4-ylethyl)amine (0.09 ml, 0.69 mmol) is added. The mixture is refluxed overnight and then allowed to cool to room temperature. The solvent is evaporated and the residue is purified by flash chromatography, eluting with CH₂Cl₂:MeOH 98:2. 0.03 g of the final compound is isolated. Yield=43%.

m.p. 184.5-185.3° C.

¹H NMR (300 MHz, METHANOL-D4) d ppm 2.69 (t, J=6.87 Hz, 2H) 3.22 (m, 4H) 3.76 (m, 16H) 4.10 (t, J=6.10 Hz, 2H) 4.79 (m, 2H) 8.52 (s, 1H)

### Example 76

### 5-Morpholin-4-yl-N-pentyl-1,4-dihydro-2H-pyrano[4″,3″:4′,5′]pyrido[3′,2′:4,5]thieno[3,2-d]pyrimidin-8-amine

Obtained (20%) following the experimental procedure described in Example 75 using n-pentylamine instead of (2-morpholin-4-yl-ethyl)-amine.

¹H NMR (300 MHz, DMSO-D6) δ ppm 0.87 (t, J=6.71 Hz, 3H) 1.29 (m, 4H) 1.61 (m, 2H) 3.16 (m, 4H) 3.51 (m, 3H) 3.75 (m, 5H) 4.03 (t, J=5.95 Hz, 2H) 4.65 (m, 2H) 7.82 (s, 1H) 8.55 (s, 1H)

### Example 77

### N-(2-Morpholin-4-ylethyl)-5-pyrrolidin-1-yl-1,4-dihydro-2H-pyrano[4″,3″:4′,5′]pyrido[3′,2′:4,5]thieno[3,2-d]pyrimidin-8-amine

8-Chloro-5-(pyrrolidin-1-yl)-1,4-dihydro-2H-pyrano[4″,3″:4′,5′]pyrido[3′,2′:4,5]thieno[3,2-d]pyrimidine (0.15 g, 0.43 mmol, see Preparation 34) is suspended in ethanol (10 ml) and (2-morpholin-4-ylethyl)amine (0.28 ml, 2.16 mmol) is added. The mixture is refluxed overnight and then allowed to cool to room temperature. The solvent is evaporated and the residue is redissolved with dichloromethane. This organic phase is washed with NaOH 1N and brine, dried over magnesium sulfate, filtrated and evaporated. The resulting material is purified by flash chromatography, eluting with dichloromethane, CH₂Cl₂:MeOH 99.5:0.5 and finally CH₂Cl₂:MeOH 98:2. 0.12 g of the final compound are isolated. Yield=63%.

m.p. 188.6-191.0° C.

¹H NMR (300 MHz, DMSO-D6) δ ppm 1.88 (m, 4H) 2.44 (m, 4H) 2.55 (m, 2H) 3.56 (m, 12H) 3.97 (t, J=5.80 Hz, 2H) 4.79 (s, 2H) 7.48 (t, J=5.49 Hz, 1H) 8.50 (s, 1H)

### Example 78

### N-Pentyl-5-pyrrolidin-1-yl-1,4-dihydro-2H-pyrano[4″,3″:4′,5′]pyrido[3′,2′:4,5]thieno[3,2-d]pyrimidin-8-amine

Obtained (87%) following the experimental procedure described in Example 77 using n-pentylamine instead of (2-morpholin-4-yl-ethyl)-amine.

m.p. 151.4-153.6° C.

¹H NMR (300 MHz, DMSO-D6) δ ppm 0.87 (t, J=6.71 Hz, 3H) 1.31 (m, 4H) 1.60 (m, 2H) 1.87 (m, 4H) 3.49 (m, 8H) 3.96 (t, J=5.80 Hz, 2H) 4.77 (s, 2H) 7.53 (t, J=5.80 Hz, 1H) 8.47 (s, 1H)

### Example 79

### N-Benzyl-5-pyrrolidin-1-yl-1,4-dihydro-2H-pyrano[4″,3″:4′,5′]pyrido[3′,2′:4,5]thieno[3,2-d]pyrimidin-8-amine

Obtained (55%) following the experimental procedure described in Example 77 using benzylamine instead of (2-morpholin-4-yl-ethyl)-amine.

m.p. 254.2-254.9° C.

¹H NMR (300 MHz, DMSO-D6) δ ppm 1.89 (m, 4H) 3.49 (m, 2H) 3.55 (m, 4H) 3.97 (t, J=5.95 Hz, 2H) 4.74 (d, J=5.80 Hz, 2H) 4.79 (s, 2H) 7.31 (m, 5H) 8.15 (t, J=5.80 Hz, 1H) 8.49 (s, 1H)

### Example 80

### 2-Ethyl-2-methyl-5-morpholin-4-yl-N-(pyridin-3-ylmethyl)-1,4-dihydro-2H-pyrano[4″,3″:4′,5′]pyrido[3′,2′:4,5]thieno[3,2-d]pyrimidin-8-amine

8-Chloro-2-ethyl-2-methyl-5-morpholin-4-yl-1,4-dihydro-2H-pyrano[4″,3″:4′,5′]pyrido[3′,2′:4,5]thieno[3,2-d]pyrimidine (0.10 g, 0.25 mmol, see Preparation 25) is suspended in ethanol (5 ml) and pyridin-3-ylmethylamine (0.13 g, 1.23 mmol) is added. The mixture is refluxed for 24 h and then cooled to room temperature. At +5° C. a precipitate is formed, which is filtrated and washed with ethanol an ethyl ether. Once dried, it weights 0.090 g and its ¹H NMR is consistent with the final product. Yield=76%.

m.p. 240.2-241.6° C.

¹H NMR (300 MHz, DMSO-D6) δ ppm 0.92 (t, J=7.32 Hz, 3H) 1.25 (s, 3H) 1.61 (m, 2H) 3.17 (m, 4H) 3.34 (s, 2H) 3.47 (m, 2H) 3.75 (m, 2H) 4.65 (m, 2H) 4.77 (d, J=5.80 Hz, 2H) 7.35 (dd, J=7.63, 4.58 Hz, 1H) 7.77 (d, J=7.63 Hz, 1H) 8.39 (m, 1H) 8.46 (d, J=3.66 Hz, 1H) 8.60 (m, 2H)

The following examples illustrate pharmaceutical compositions according to the present invention.

### Example 81

### N⁵, N⁵,2,2-tetramethyl-N⁸-(2-morpholin-4-ylethyl)-1,4-dihydro-2H-pyrano[4″,3″:4′,5′]pyrido[3′,2′:4,5]thieno[3,2-d]pyrimidine-5,8-diamine

8-Chloro-5-dimethylamino-2,2-dimethyl-1,4-dihydro-2H-pyrano[4″,3″:4′,5′]pyrido[3′,2′:4,5]thieno[3,2-d]pyrimidine (0.07 g, 0.20 mmol, see Preparation 41) is suspended in ethanol (5 ml) and (2-morpholino-4-ylethyl)amine (0.13 ml, 1.00 mmol) is added. The mixture is refluxed for 48 h and then cooled to room temperature. The solvent is evaporated under vacuum and the residue is purified by flash chromatography, eluting first with dichloromethane and then with CH₂Cl₂:MeOH 98:2. 74 mg of the desired final product are obtained. Yield=83%.

m.p. 195.1-195.8° C.

¹H NMR (300 MHz, CHLOROFORM-D) δ ppm 1.43 (s, 6H) 2.55 (s, 4H) 2.72 (t, J=6.04 Hz, 2H) 2.98 (s, 6H) 3.58 (s, 2H) 3.74 (m, 6H) 4.80 (s, 2H) 5.56 (m, 1H) 8.70 (s, 1H)

### Example 82

### 2,2-Dimethyl-5-dimethylamino-N-(3-morpholin-4-ylpropyl)-1,4-dihydro-2H-pyrano[4″,5″:4′,5′]pyrido[3′,2′:4,5]thieno[3,2-d]pyrimidin-8-amine

Obtained (35%) from the title compound of Preparation 41 and 3-morpholin-4-ylpropylamine following the experimental procedure described at Example 81.

¹H NMR (300 MHz, CHLOROFORM-D) δ ppm 1.30 (s, 6H) 1.80 (m, 2H) 2.40 (m, 6H) 2.90 (s, 6H) 3.60 (m, 8H) 4.70 (s, 2H) 7.70 (t, 1H) 8.55 (s, 1H)

### Example 83

### N⁸-(2,3-Dimethoxybenzyl)-N⁵, N⁵,2,2-tetramethyl-1,4-dihydro-2H-pyrano[4″,3″:4′,5′]pyrido[3′,2′:4,5]thieno[3,2-d]pyrimidine-5,8-diamine

Obtained (96%) from the title compound of Preparation 41 and (2,3-dimethoxybenzyl)amine following the experimental procedure described in Example 81.

m.p. 89.9-90.7° C.

¹H NMR (300 MHz, CHLOROFORM-D) δ ppm 1.42 (s, 6H) 3.00 (m, 6H) 3.58 (s, 2H) 3.89 (s, 3H) 3.94 (s, 3H) 4.79 (s, 2H) 4.90 (d, J=5.77 Hz, 2H) 5.17 (m, 1H) 6.90 (dd, J=7.14, 2.75 Hz, 1H) 7.05 (m, 2H) 8.73 (s, 1H)

### Example 84

### N⁵, N⁵,2,2-Tetramethyl-N⁸-(pyridin-4-ylmethyl)-1,4-dihydro-2H-pyrano[4″,3″:4′,5′]pyrido[3′,2′:4,5]thieno[3,2-d]pyrimidine-5,8-diamine

Obtained (50%) from the title compound of Preparation 41 and (pyridin-4-ylmethyl)amine following the experimental procedure described in Example 81.

m.p. 202.0-203.8° C.

¹H NMR (300 MHz, CHLOROFORM-D) δ ppm 1.43 (s, 6H) 3.00 (s, 6H) 3.59 (s, 2H) 4.80 (s, 2H) 4.94 (d, J=6.32 Hz, 2H) 5.12 (s, 1H) 7.30 (m, 2H) 8.58 (m, 2H) 8.70 (s, 1H)

### Example 85

### N⁵,N⁵,2,2-Tetramethyl-N⁸-(pyridin-3-ylmethyl)-1,4-dihydro-2H-pyrano[4″,3″:4′,5′]pyrido[3′,2′:4,5]thieno[3,2-d]pyrimidine-5,8-diamine

Obtained (92%) from the title compound of Preparation 41 and (pyridin-3-ylmethyl)amine following the experimental procedure described in Example 81.

m.p. 250.4-252.2° C.

¹H NMR (300 MHz, CHLOROFORM-D) δ ppm 1.43 (s, 6H) 3.00 (m, 6H) 3.59 (s, 2H) 4.80 (s, 2H) 4.92 (d, J=6.04 Hz, 2H) 5.03 (m, 1H) 7.29 (dd, J=7.55, 5.08 Hz, 1H) 7.77 (m, 1H) 8.56 (dd, J=4.81, 1.79 Hz, 1H) 8.69 (d, J=1.92 Hz, 1H) 8.73 (s, 1H)

### Example 86

### N⁵,N⁵,2,2-Tetramethyl-N⁸-(pyridin-2-ylmethyl)-1,4-dihydro-2H-pyrano[4″,3″:4′,5′]pyrido[3′,2′:4,5]thieno[3,2-d]pyrimidine-5,8-diamine

Obtained (83%) from the title compound of Preparation 41 and (pyridin-2-ylmethyl)amine following the experimental procedure described in Example 81.

m.p. 216.9-217.8° C.

¹H NMR (300 MHz, CHLOROFORM-D) δ ppm 1.43 (s, 6H) 2.99 (s, 6H) 3.59 (s, 2H) 4.80 (s, 2H) 4.96 (d, J=4.67 Hz, 2H) 6.26 (t, J=4.67 Hz, 1H) 7.25 (m, 1H) 7.37 (d, J=7.97 Hz, 1H) 7.70 (m, 1H) 8.63 (d, J=4.94 Hz, 1H) 8.74 (s, 1H)

### Example 87

### 1-(3-{[5-Dimethylamino)-2,2-dimethyl-1,4-dihydro-2H-pyrano[4″,3″:4′,5′]pyrido[3′,2′:4,5]thieno[3,2-d]pyrimidin-8-yl]amino}propyl)pyrrolilydin-2-one

Obtained (92%) from the title compound of Preparation 41 and 1-(3-aminopropyl)-pyrrolidin-2-one following the experimental procedure described in Example 81.

m.p. 198.4-199.5° C.

¹H NMR (300 MHz, CHLOROFORM-D) δ ppm 1.30 (s, 6H) 1.80 (m, 2H) 1.90 (m, 2H) 2.2 (t, 2H) 2.90 (s, 6H) 3.25 (t, 2H) 3.30 (s, 2H) 3.35 (t, 2H) 3.45 (m, 2H) 4.70 (s, 2H) 7.60 (t, 1H) 8.55 (s, 1H)

### Example 88

### N-(2,3-Dimethoxybenzyl)-5-(pyrrolidin-1-yl)-2,2-dimethyl-1,4-dihydro-2H-pyrano[4″,3″:4′,5′]pyrido[3′,2′:4,5]thieno[3,2-d]pyrimidine-8-amine

8-Chloro-2,2-dimethyl-5-(pyrrolidin-1-yl)-1,4-dihydro-2H-pyrano[4″,3″:4′,5′]pyrido[3′,2′:4,5]thieno[3,2-d]pyrimidine (0.08 g, 0.21 mmol, see Preparation 24) is suspended in ethanol (5 ml) and (2,3-dimethoxybenzyl)amine (0.16 ml, 1.07 mmol) is added. The mixture is refluxed for 24 h and then allowed to cool to room temperature. The solvent is evaporated under vacuum and the residue is purified by chromatography, eluting first with CH₂Cl₂ and then with CH₂Cl₂:MeOH 99:1. 85 mg of the desired final product are obtained. Yield=79%.

m.p. 166.0-167.5° C.

¹H NMR (300 MHz, DMSO-D6) δ ppm 1.32 (s, 6H) 1.88 (m, 4H) 3.33 (d, J=7.02 Hz, 3H) 3.60 (m, 4H) 3.78 (m, 6H) 4.74 (d, J=5.80 Hz, 2H) 4.83 (s, 2H) 6.83 (dd, J=7.17, 1.98 Hz, 1H) 6.96 (m, 1H) 8.01 (t, J=5.80 Hz, 1H) 8.48 (s, 1H)

### Example 89

### 2,2-Dimethyl-N-(pyridin-3-ylmethyl)-5-pyrrolidin-1-yl-1,4-dihydro-2H-pyrano[4″,3″:4′,5′]pyrido[3′,2′:4,5]thieno[3,2-d]pyrimidin-8-amine

Obtained (65%) from the title compound of Preparation 24 and (pyridine-3-ylmethyl)-amine following the experimental procedure described in Example 88.

m.p. 289.0-289.6° C.

¹H NMR (300 MHz, CHLOROFORM-D) δ ppm 1.42 (s, 6H) 1.64 (s, 4H) 1.97 (t, J=6.41 Hz, 3H) 3.55 (s, 2H) 3.63 (t, J=6.41 Hz, 3H) 4.91 (s, 2H) 4.99 (d, J=6.10 Hz, 1H) 7.29 (m, 1H) 7.76 (d, J=7.94 Hz, 1H) 8.55 (d, J=3.66 Hz, 1H) 8.69 (m, 2H)

### Example 90

### 2,2-Dimethyl-N-(pyridin-2-ylmethyl)-5-pyrrolidin-1-yl-1,4-dihydro-2H-pyrano[4″,3″:4′,5′]pyrido[3′,2′:4,5]thieno[3,2-d]pyrimidin-8-amine

Obtained (59%) from the title compound of Preparation 24 and (pyridine-2-ylmethyl)-amine following the experimental procedure described in Example 88.

m.p. 249.1-250.9° C.

¹H NMR (300 MHz, CHLOROFORM-D) δ ppm 1.42 (s, 6H) 1.62 (s, 4H) 1.98 (m, 2H) 3.56 (s, 2H) 3.66 (m, 2H) 4.90 (s, 2H) 4.95 (m, 2H) 6.18 (t, J=4.58 Hz, 1H) 7.24 (m, 1H) 7.37 (d, J=7.63 Hz, 1H) 7.70 (m, 1H) 8.62 (d, J=4.88 Hz, 1H) 8.71 (s, 1H)

### Example 91

### 2,2-Dimethyl-N-[2-(methylthio)benzyl]-5-pyrrolidin-1-yl-1,4-dihydro-2H-pyrano[4″,3″:4′,5′]pyrido[3′,2′:4,5]thieno[3,2-d]pyrimidin-8-amine

Obtained (62%) from the title compound of Preparation 24 and [2-(methylthio)benzyl]amine following the experimental procedure described in Example 88.

m.p. 175.8-176.8° C.

¹H NMR (300 MHz, CHLOROFORM-D) δ ppm 1.41 (s, 6H) 1.96 (m, 4H) 2.51 (d, J=5.19 Hz, 3H) 3.55 (s, 2H) 3.62 (m, 4H) 4.89 (s, 2H) 4.94 (d, J=5.80 Hz, 2H) 7.16 (m, 2H) 7.29 (m, 1H) 7.43 (d, J=7.32 Hz, 1H) 8.70 (s, 1H)

### Example 92

### 2,2-Dimethyl-N-[4-(methylsulfonyl)benzyl]-5-pyrrolidin-1-yl-1,4-dihydro-2H-pyrano[4″,3″:4′,5′]pyrido[3′,2′:4,5]thieno[3,2-d]pyrimidin-8-amine

Obtained (54%) from the title compound of Preparation 24 and [4-(methylsulfonyl)benzyl]amine following the experimental procedure described in Example 88.

m.p. 323.9-325.6° C.

¹H NMR (300 MHz, DMSO-D6) δ ppm 1.32 (s, 6H) 1.90 (s, 4H) 3.18 (s, 2H) 3.33 (d, J=7.02 Hz, 3H) 3.41 (m, 2H) 3.61 (s, 4H) 4.83 (m, 3H) 7.60 (d, J=8.55 Hz, 2H) 7.88 (d, J=8.55 Hz, 2H) 8.50 (s, 1H)

### Example 93

### 4-{[(2,2-Dimethyl-5-pyrrolidin-1-yl-1,4-dihydro-2H-pyrano[4″,3″:4′,5′]pyrido[3′,2′:4,5]thieno[3,2-d]pyrimidin-8-yl)amino]methyl}benzenesulfonamide

Obtained (27%) from the title compound of Preparation 24 and 4-(aminomethyl)benzenesulfonamide following the experimental procedure described in Example 88.

m.p. 294.9-295.3° C.

¹H NMR (300 MHz, DMSO-D6) δ ppm 1.31 (s, 6H) 1.86 (d, J=16.79 Hz, 4H) 3.36 (m, 4H) 3.60 (s, 4H) 4.83 (s, 3H) 7.31 (s, 1H) 7.53 (s, 2H) 7.76 (s, 2H) 8.21 (d, J=5.49 Hz, 1H) 8.49 (s, 1H)

### Example 94

### 1-{3-[(2,2-Dimethyl-5-pyrrolidin-1-yl-1,4-dihydro-2H-pyrano[4″,3″:4′,5′]pyrido[3′,2′:4,5]thieno[3,2-d]pyrimidin-8-yl)amino]propyl}pyrrolidin-2-one

Obtained (89%) from the title compound of Preparation 24 and 1-(3-aminopropyl)-pyrrolidin-2-one following the experimental procedure described in Example 88.

m.p. 216.6-217.0° C.

¹H NMR (400 MHz, CHLOROFORM-D) δ ppm 1.4 (s, 6H) 1.9 (m, 2H) 2.0 (m, 4H) 2.1 (m, 2H) 2.5 (t, J=8.2 Hz, 2H) 3.4 (m, 4H) 3.5 (s, 2H) 3.6 (m, 6H) 4.9 (s, 2H) 6.2 (t, J=6.3 Hz, 1H) 8.6 (s, 1H)

### Example 95

### N-[2-(1H-imidazol-4-yl)ethyl]-2,2-dimethyl-5-pyrrolidin-1-yl-1,4-dihydro-2H-pyrano[4″,3″:4′,5′]pyrido[3′,2′:4,5]thieno[3,2-d]pyrimidin-8-amine

Obtained (33%) from the title compound of Preparation 24 and [2-(1H-imidazol-4-yl)ethyl]-amine following the experimental procedure described in Example 88.

¹H NMR (400 MHz, DMSO-D6) δ ppm 1.3 (s, 6H) 2.8 (s, 2H) 3.3 (m, 6H) 3.4 (s, 2H) 3.6 (m, 4H) 3.7 (m, 2H) 4.8 (s, 2H) 7.5 (s, 1H) 7.6 (t, J=5.9 Hz, 1H) 8.5 (s, 1H)

### Example 96

### Ethyl 4-{2-[(2,2-dimethyl-5-pyrrolidin-1-yl-1,4-dihydro-2H-pyrano[4″,3″:4′,5′]pyrido[3′,2′:4,5]thieno[3,2-d]pyrimidin-8-yl)amino]ethyl}piperazine-1-carboxylate

Obtained (63%) from the title compound of Preparation 24 and ethyl 4-(2-aminoethyl)-piperazine-1-carboxylate following the experimental procedure described in Example 88.

m.p. 97.8-99.1° C.

¹H NMR (400 MHz, CHLOROFORM-D) δ ppm 1.3 (t, J=7.0 Hz, 3H) 1.4 (s, 6H) 2.0 (m, 4H) 2.5 (m, 4H) 2.7 (t, J=5.9 Hz, 2H) 3.5 (m, 4H) 3.6 (s, 2H) 3.6 (m, 4H) 3.7 (q, J=5.3 Hz, 2H) 4.2 (q, J=7.0 Hz, 2H) 4.9 (s, 2H) 5.4 (t, J=4.5 Hz, 1H) 8.7 (s, 1H)

### Example 97

### 2,2-Dimethyl-N-[2-(4-methyl piperazin-1-yl)ethyl]-5-pyrrolidin-1-yl-1,4-dihydro-2H-pyrano[4″,3″:4′,5′]pyrido[3′,2′:4,5]thieno[3,2-d]pyrimidin-8-amine

Obtained (67%) from the title compound of Preparation 24 and 2-(4-methylpiperazin-1-yl)-ethylamine following the experimental procedure described in Example 88.

m.p. 97.5-98.8° C.

¹H NMR (400 MHz, CHLOROFORM-D) δ ppm 1.4 (s, 6H) 2.0 (m, 4H) 2.3 (s, 5H) 2.7 (t, J=5.9 Hz, 4H) 3.6 (s, 4H) 3.6 (m, 4H) 3.7 (m, 4H) 4.9 (s, 2H) 5.6 (m, 1H) 8.7 (s, 1H)

### Example 98

### 2,2-Dimethyl-5-morpholin-4-yl-N-(quinolin-3-ylmethyl)-1,4-dihydro-2H-pyrano[4″,3″:4′,5′]pyrido[3′,2′:4,5]thieno[3,2-d]pyrimidin-8-amine

Obtained (56%) from the title compound of Preparation 8 and quinolin-3-yl-methylamine following the experimental procedure described in Example 55.

m.p. 271.9-272.6° C.

¹H NMR (400 MHz, CHLOROFORM-D) δ ppm 1.30 (s, 6H) 3.20 (m, 4H) 3.5 (s, 2H) 3.75 (m, 4H) 4.70 (s, 2H) 4.95 (d, 2H) 7.60 (t, 1H) 7.7 (t, 1H) 7.95 (d, 1H) 8.05 (d, 1H) 8.25 (s, 1H) 8.45 (t, 1H) 8.60 (s, 1H) 9.0 (s, 1H)

### Example 99

### 1-{3-[(2,2-Dimethyl-5-morpholin-4-yl-1,4-dihydro-2H-pyrano[4″,3″:4′,5′]pyrido[3′,2′:4,5]thieno[3,2-d]pyrimidin-8-yl)amino]propyl}pyrrolidin-2-one

Obtained (80%) from the title compound of Preparation 8 and 1-(3-aminopropyl)-pyrrolidin-2-one following the experimental procedure described in Example 55.

m.p. 215.9-216.7° C.

¹H NMR (400 MHz, DMSO-D6) δ ppm 1.3 (s, 6H) 1.8 (m, 2H) 1.9 (m, 2H) 2.2 (t, J=8.2 Hz, 2H) 3.2 (m, 4H) 3.3 (m, 2H) 3.4 (m, 2H) 3.5 (m, 4H) 3.8 (m, 4H) 4.7 (s, 2H) 7.7 (t, J=5.5 Hz, 1H) 8.6 (s, 1H)

### Example 100

### 2-[(2,2-Dimethyl-5-morpholin-4-yl-1,4-dihydro-2H-pyrano[4″,3″:4′,5′]pyrido[3′,2′:4,5]thieno[3,2-d]pyrimidin-8-yl)(2-morpholin-4-ylethyl)amino]ethanol

Obtained (41%) from the title compound of Preparation 8 and 2-(2-morpholin-4-ylethylamino)-ethanol following the experimental procedure described in Example 55.

m.p. 111.9-112.6° C.

¹H NMR (400 MHz, DMSO-D6) δ ppm 1.3 (s, 6H) 2.5 (m, 4H) 2.6 (t, J=6.8 Hz, 2H) 3.2 (m, 4H) 3.5 (s, 2H) 3.6 (m, 4H) 3.7 (m, 6H) 3.9 (t, J=6.1 Hz, 2H) 3.9 (t, J=6.8 Hz, 2H) 4.7 (s, 2H) 5.0 (t, J=5.7 Hz, 1H) 8.6 (s, 1H)

### Example 101

### 2,2-Dimethyl-5-morpholin-4-yl-N-(2-morpholin-4-ylethyl)-N-(pyridin-3-ylmethyl)-1,4-dihydro-2H-pyrano[4″,3″:4′,5′]pyrido[3′,2′:4,5]thieno[3,2-d]pyrimidin-8-amine

Obtained (14%) from the title compound of Preparation 8 and (2-morpholin-4-ylethyl)-pyridin-2-ylmethylamine following the experimental procedure described in Example 55.

m.p. 149.8-150.3° C.

¹H NMR (400 MHz, DMSO-D6) δ ppm 1.3 (s, 6H) 2.4 (s, 6H) 2.7 (t, J=6.8 Hz, 2H) 3.2 (m, 4H) 3.5 (d, J=8.7 Hz, 4H) 3.8 (m, 4H) 3.9 (t, J=6.6 Hz, 2H) 4.7 (s, 2H) 5.2 (s, 2H) 7.3 (dd, J=7.5, 4.6 Hz, 1H) 7.7 (d, J=8.3 Hz, 1H) 8.5 (m, 1H) 8.6 (d, J=1.7 Hz, 1H) 8.6 (s, 1H)

### Example 102

### 2,2-Dimethyl-5-morpholin-4-yl-N-(2-morpholin-4-yl-2-oxoethyl)-1,4-dihydro-2H-pyrano[4″,3″:4′,5′]pyrido[3′,2′:4,5]thieno[3,2-d]pyrimidin-8-amine

Obtained (78%) from the title compound of Preparation 8 and 2-amino-1-morpholin-4-yl-ethanone following the experimental procedure described in Example 55.

m.p. 209.0-209.8° C.

¹H NMR (400 MHz, DMSO-D6) δ ppm 1.3 (s, 6H) 3.2 (s, 4H) 3.5 (s, 2H) 3.5 (s, 2H) 3.6 (s, 3H) 3.6 (s, 2H) 3.8 (m, 4H) 3.9 (m, 1H) 4.4 (m, 2H) 4.7 (s, 2H) 7.9 (m, 1H) 8.6 (s, 1H)

### Example 103

### N²-(2,2-Dimethyl-5-morpholin-4-yl-1,4-dihydro-2H-pyrano[4″,3″:4′,5′]pyrido[3′,2′:4,5]thieno[3,2-d]pyrimidin-8-yl)-N¹-(2-morpholin-4-ylethyl)glycinamide

Obtained (24%) from the title compound of Preparation 8 and 2-amino-N-(2-morpholin-4-ylethyl)acetamide following the experimental procedure described in Example 55.

¹H NMR (300 MHz, CHLOROFORM-D) δ ppm 1.4 (s, 6H) 2.4 (m, 4H) 2.5 (t, J=6.0 Hz, 2H) 3.3 (m, 4H) 3.4 (q, J=5.8 Hz, 2H) 3.6 (s, 6H) 3.9 (m, 4H) 4.3 (d, J=5.2 Hz, 2H) 4.8 (s, 2H) 5.6 (m, 1H) 6.7 (s, 1H) 8.7 (s, 1H)

### Example 104

### 2,2′-[(2,2-Dimethyl-5-morpholin-4-yl-1,4-dihydro-2H-pyrano[4″,3″:4′,5′]pyrido[3′,2′:4,5]thieno[3,2-d]pyrimidin-8-yl)imino]diethanol

Obtained (48%) from the title compound of Preparation 8 and 2-(2-hydroxyethylamino)-ethanol following the experimental procedure described in Example 55.

¹H NMR (300 MHz, CHLOROFORM-D) δ ppm 1.4 (s, 6H) 3.3 (m, 4H) 3.6 (s, 2H) 3.7 (br. s., 2H) 3.9 (m, 4H) 4.0 (t, J=3.2 Hz, 8H) 4.8 (s, 2H) 8.6 (s, 1H)

### Example 105

### N⁵,2,2-Trimethyl-N⁸-(2-morpholin-4-ylethyl)-1,4-dihydro-2H-pyrano[4″,3″:4′,5′]pyrido[3′,2′:4,5]thieno[3,2-d]pyrimidin-5,8-diamine

N⁵-Benzyl-N⁵,2,2-trimethyl-N⁸-(2-morpholin-4-ylethyl)-1,4-dihydro-2H-pyrano[4″,3″:4′,5′]pyrido[3′,2′:4,5]thieno[3,2-d]pyrimidin-5,8-diamine (0.20 g, 0.39 mmol, see Preparation 46) is dissolved in toluene and aluminum chloride is portionwise added. This reaction mixture is refluxed for 2 h. Once the reaction is over, the reaction mixture is diluted with ethyl acetate and washed twice with water. The aqueous phase is then basified with NaOH 2N and extracted with dichloromethane. This organic phase is washed with water and brine, dried over magnesium sulfate, filtered and evaporated under reduced pressure. The residue is purified by flash chromatography, eluting first with dichloromethane, then with dichloromethane/methanol 99:1 and finally with dichloromethane/methanol 98:2. 20 mg of the final compound are isolated. Its ¹HNMR is consistent with the desired final compound. Yield=12%.

¹H NMR (300 MHz, CHLOROFORM-D) δ ppm 1.4 (s, 6H) 2.6 (s, 4H) 2.7 (t, J=5.9 Hz, 2H) 3.2 (d, J=4.9 Hz, 3H) 3.5 (s, 2H) 3.8 (dd, J=9.6, 4.9 Hz, 6H) 4.2 (d, J=4.9 Hz, 1H) 4.6 (s, 2H) 5.5 (s, 1H) 8.7 (s, 1H)

### Example 106

### N⁵,2,2-Trimethyl-N⁸-(pyridin-3-ylmethyl)-1,4-dihydro-2H-pyrano[4″,3″:4′,5′]pyrido[3′,2′:4,5]thieno[3,2-d]pyrimidin-5,8-diamine

Obtained (47%) from the title compound of Preparation 47 following the experimental procedure described in Example 105.

¹H NMR (300 MHz, CHLOROFORM-D) δ ppm 1.4 (s, 6H) 3.1 (d, J=4.9 Hz, 3H) 4.4 (d, J=4.9 Hz, 1H) 4.6 (s, 2H) 4.9 (d, J=5.8 Hz, 2H) 4.9 (d, J=5.8 Hz, 2H) 5.5 (t, J=5.9 Hz, 1H) 7.3 (m, 1H) 7.7 (d, J=7.7 Hz, 1H) 8.5 (d, J=4.1 Hz, 1H) 8.6 (s, 1H) 8.7 (s, 1H)

### Example 107

### 1-[3-({5-Methylamino-2,2-dimethyl-1,4-dihydro-2H-pyrano[4″,3″:4′,5′]pyrido[3′,2′:4,5]thieno[3,2-d]pyrimidin-8-yl}amino)propyl]pyrrolidin-2-one

Obtained (23%) from the title compound of Preparation 48 following the experimental procedure described in Example 105.

¹H NMR (300 MHz, CHLOROFORM-D) δ ppm 1.4 (s, 6H) 1.9 (m, 2H) 2.1 (m, 2H) 2.5 (t, J=8.2 Hz, 2H) 3.1 (d, J=4.7 Hz, 3H) 3.5 (m, 6H) 3.7 (q, J=6.2 Hz, 2H) 4.2 (d, J=4.4 Hz, 1H) 4.6 (s, 2H) 6.3 (s, 1H) 8.6 (s, 1H)

### Example 108

### N⁵,2,2-Trimethyl-N⁸-(2-morpholin-4-ylethyl)-N⁸-(pyridin-3-ylmethyl)-1,4-dihydro-2H-pyrano[4″,3″:4′,5′]pyrido[3′,2′:4,5]thieno[3,2-d]pyrimidin-5,8-diamine

Obtained (16%) from the title compound of Preparation 49 following the experimental procedure described in Example 105.

¹H NMR (300 MHz, CHLOROFORM-D) δ ppm 1.4 (s, 6H) 2.5 (s, 4H) 2.8 (s, 2H) 3.1 (d, J=4.7 Hz, 3H) 3.5 (s, 2H) 3.7 (s, 4H) 3.9 (s, 2H) 4.3 (q, J=4.6 Hz, 1H) 4.6 (s, 2H) 5.2 (s, 2H) 7.2 (m, 1H) 7.7 (d, J=8.0 Hz, 1H) 8.5 (d, J=4.4 Hz, 1H) 8.6 (s, 1H) 8.6 (s, 1H)

### Composition Examples

### Composition Example 1

Preparation of tablets
Formulation:

| | |
|---|---|
| Compound of the present invention | 5.0 mg |
| Lactose | 113.6 mg |
| Microcrystalline cellulose | 28.4 mg |
| Light silicic anhydride | 1.5 mg |
| Magnesium stearate | 1.5 mg |

Using a mixer machine, 15 g of the compound of the present invention are mixed with 340.8 g of lactose and 85.2 g of microcrystalline cellulose. The mixture is subjected to compression moulding using a roller compactor to give a flake-like compressed material. The flake-like compressed material is pulverised using a hammer mill, and the pulverised material is screened through a 20 mesh screen. A 4.5 g portion of light silicic anhydride and 4.5 g of magnesium stearate are added to the screened material and mixed. The mixed product is subjected to a tablet making machine equipped with a die/punch system of 7.5 mm in diameter, thereby obtaining 3,000 tablets each having 150 mg in weight.

### Composition Example 2

Preparation of coated tablets
Formulation:

| | |
|---|---|
| Compound of the present invention | 5.0 mg |
| Lactose | 95.2 mg |
| Corn starch | 40.8 mg |
| Polyvinylpyrrolidone K25 | 7.5 mg |
| Magnesium stearate | 1.5 mg |
| Hydroxypropylcellulose | 2.3 mg |
| Polyethylene glycol 6000 | 0.4 mg |
| Titanium dioxide | 1.1 mg |
| Purified talc | 0.7 mg |

Using a fluidised bed granulating machine, 15 g of the compound of the present invention are mixed with 285.6 g of lactose and 122.4 g of corn starch. Separately, 22.5 g of polyvinylpyrrolidone is dissolved in 127.5 g of water to prepare a binding solution. Using a fluidised bed granulating machine, the binding solution is sprayed on the above mixture to give granulates. A 4.5 g portion of magnesium stearate is added to the obtained granulates and mixed. The obtained mixture is subjected to a tablet making machine equipped with a die/punch biconcave system of 6.5 mm in diameter, thereby obtaining 3,000 tablets, each having 150 mg in weight.

Separately, a coating solution is prepared by suspending 6.9 g of hydroxypropylmethyl-cellulose 2910, 1.2 g of polyethylene glycol 6000, 3.3 g of titanium dioxide and 2.1 g of purified talc in 72.6 g of water. Using a High Coated, the 3,000 tablets prepared above are coated with the coating solution to give film-coated tablets, each having 154.5 mg in weight.

### Composition Example 3

Preparation of capsules
Formulation:

| | |
|---|---|
| Compound of the present invention | 5.0 mg |
| Lactose monohydrate | 200 mg |
| Colloidal silicon dioxide | 2 mg |
| Corn starch | 20 mg |
| Magnesium stearate | 4 mg |

25 g of active compound, 1 Kg of lactose monohydrate, 10 g of colloidal silicon dioxide, 100 g of corn starch and 20 g of magnesium stearate are mixed. The mixture is sieved through a 60 mesh sieve, and then filled into 5,000 gelatine capsules.

### Composition Example 4

Preparation of a cream
Formulation:

| | |
|---|---|
| Compound of the present invention | 1% |
| Cetyl alcohol | 3% |
| Stearyl alcohol | 4% |
| Gliceryl monostearate | 4% |
| Sorbitan monostearate | 0.8% |
| Sorbitan monostearate POE | 0.8% |
| Liquid vaseline | 5% |
| Methylparaben | 0.18% |
| Propylparaben | 0.02% |
| Glycerine | 15% |
| Purified water csp. | 100% |

An oil-in-water emulsion cream is prepared with the ingredients listed above, using conventional methods.

1-11. (canceled)

12. A pharmaceutical composition comprising a pyrido[3′,2′:4,5]thieno[3,2-d]pyrimidine derivative of formula (I) 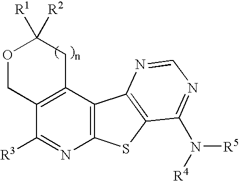
wherein
n is an integer chosen from 0 and 1;
R¹ and R² are independently chosen from hydrogen atoms and C₁₋₄ alkyl groups;
R³ is chosen from alkyl, amino, monoalkylamino, dialkylamino, aryl, heteroaryl and saturated N-containing heterocyclyl groups bound through the nitrogen atom to the pyridine ring, wherein each group is optionally substituted by one or more substituents chosen from the group consisting of halogen atoms and alkyl, alkoxyalkyl, arylalkyl, R⁶OCO—, alkoxy, R⁶R⁷N—CO—, —CN, —CF₃, —NR⁶R⁷, —SR⁶ and —SO₂NH₂ groups, wherein R⁶ and R⁷ are independently chosen from hydrogen atoms and C₁₋₄ alkyl groups;
R⁴ and R⁵ are independently chosen from the group consisting of hydrogen atoms, alkyl groups and groups of formula (II): 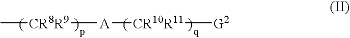
wherein p and q are integers chosen from 1, 2 and 3; A is a direct bond or a group chosen from —CONR¹²—, —NR¹²CO—, —O— —COO—, —OCO—, —NR¹²COO—, —OCONR¹²—, —NR¹²CONR¹³—, —S—, —SO—, —SO₂—, —COS— and —SCO—; and G² is a group chosen from aryl, heteroaryl and heterocyclyl; wherein the alkyl groups and the group G² are optionally substituted by one or more substuents chosen from the group consisting of halogen atoms and alkyl, alkoxyalkyl, arylalkyl, R¹⁴OCO—, hydroxy, alkoxy, oxo, R¹⁴R¹⁵N—CO—, —CN, —CF₃, —NR¹⁴R¹⁵, —SR¹⁴ and —SO₂NH₂ groups; wherein the groups R⁸, R⁹, R¹⁰, R¹¹, R¹², R¹³, R¹⁴ and R¹⁵ are independently chosen from hydrogen atoms and C₁₋₄ alkyl groups;
a pharmaceutically acceptable salt thereof, or an N-oxide thereof in admixture with a pharmaceutically acceptable diluent or carrier.

13. A method for treating a subject afflicted with a pathological condition or disease susceptible to amelioration by inhibition of phosphodiesterase comprising administering to said subject an effective amount of a pyrido[3′,2′:4,5]thieno[3,2-d]pyrimidine derivative of formula (I) 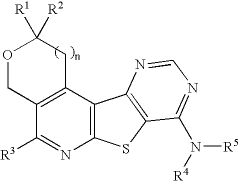
wherein
n is an integer chosen from 0 and 1;
R¹ and R² are independently chosen from hydrogen atoms and C₁₋₄ alkyl groups;
R³ is chosen from alkyl, amino, monoalkylamino, dialkylamino, aryl, heteroaryl and saturated N-containing heterocyclyl groups bound through the nitrogen atom to the pyridine ring, wherein each group is optionally substituted by one or more substituents chosen from the group consisting of halogen atoms and alkyl, alkoxyalkyl, arylalkyl. R⁶OCO—, alkoxy, R⁶R⁷N—CO—, —CN, —CF₃, —NR⁶R⁷, —SR⁶ and —SO₂NH₂ groups, wherein R⁶ and R⁷ are independently chosen from hydrogen atoms and C₁₋₄ alkyl groups;
R⁴ and R⁵ are independently chosen from the group consisting of hydrogen atoms, alkyl groups and groups of formula (II): 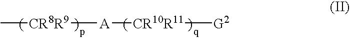
wherein p and q are integers chosen from 1, 2 and 3; A is a direct bond or a group chosen from —CONR¹²—, —NR¹²CO—, —O—, —COO—, —OCO—, —NR¹²COO—, —OCONR¹²—, —NR¹²CONR¹³—, —S, —SO—, —SO₂—, —COS— and —SCO—; and G² is a group chosen from aryl, heteroaryl and heterocyclyl; wherein the alkyl groups and the group G² are optionally substituted by one or more substituents chosen from the group consisting of halogen atoms and alkyl, alkoxyalkyl, arylalkyl, R¹⁴OCO—, hydroxy, alkoxy, oxo, R¹⁴R¹⁵N—CO—, —CN, —CF₃, —NR¹⁴R¹⁵, —SR¹⁴ and —SO₂NH₂ groups; wherein the groups R⁸, R⁹, R¹⁰, R¹¹, R¹², R¹³, R¹⁴ and R¹⁵ are independently chosen from hydrogen atoms and C₁₋₄ alkyl groups;
a pharmaceutically acceptable salt thereof, or an N-oxide thereof.

14. A method according to claim 13, wherein the pathological condition or disease is chosen from asthma, chronic obstructive pulmonary disease, rheumatoid arthritis, atopic dermatitis, psoriasis, and irritable bowel disease.

15. A product for simultaneous, separate or sequential use in the treatment of the human or animal body comprising:
a pyrido[3′,2′:4,5]thieno[3,2-d]pyrimidine derivative of formula (I) 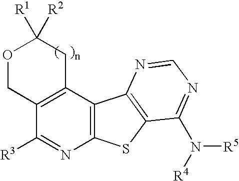
wherein
n is an integer chosen from 0 and 1;
R¹ and R² are independently chosen from hydrogen atoms and C₁₋₄ alkyl groups;
R³ is chosen from alkyl, amino, monoalkylamino, dialkylamino, aryl, heteroaryl and saturated N-containing heterocyclyl groups bound through the nitrogen atom to the pyridine ring, wherein each group is optionally substituted by one or more substituents chosen from the group consisting of halogen atoms and alkyl, alkoxyalkyl, arylalkyl, R⁶OCO—, alkoxy, R⁶R⁷N—CO—, —CN, —CF₃, —NR⁶R⁷, —SR⁶ and —SO₂NH₂ groups, wherein R⁶ and R⁷ are independently chosen from hydrogen atoms and C₁₋₄ alkyl groups;
R⁴ and R⁵ are independently chosen from the group consisting of hydrogen atoms, alkyl groups and groups of formula (II): 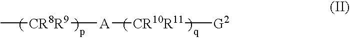
wherein p and q are integers chosen from 1, 2 and 3; A is a direct bond or a group chosen from —CONR¹²—, —NR¹²CO—, —O—, —COO—, —OCO—, —NR¹²COO—, —OCONR¹²—. —NR¹²CONR¹³—, —S—, —SO—, —SO₂—, —COS— and —SCO—; and G² is a group chosen from aryl, heteroaryl and heterocyclyl; wherein the alkyl groups and the group G² are optionally substituted by one or more substuents chosen from the group consisting of halogen atoms and alkyl, alkoxyalkyl, arylalkyl, R¹⁴OCO—, hydroxy, alkoxy, oxo, R¹⁴R¹⁵N—CO—, —CN, —CF₃, —NR¹⁴R¹⁵—SR¹⁴ and —SO₂NH₉ groups; wherein the groups R⁸, R⁹, R¹⁰, R¹¹, R¹², R¹³, R¹⁴ and R¹⁵ are independently chosen from hydrogen atoms and C₁₋₄ alkyl groups;
a pharmaceutically acceptable salt thereof, or N-oxide thereof; and
at least one compound chosen from:
(a) steroids,
(b) immunosuppressive agents,
(c) T-cell receptor blockers, and
(d) antiinflammatory drugs.
